(19) United States
(12) Patent Application Publication
(10) Pub. No.: US 2024/0261582 A1
(43) Pub. Date: Aug. 8, 2024

(54) WIRELESS POWER TRANSFER

(71) Applicant: XII Medical, Inc., Union City, CA (US)

(72) Inventors: Anthony V. Caparso, North Ridgeville, OH (US); Steven M. Young, Ann Arbor, MI (US); Paul F. Wittibschlager, Macedonia, OH (US); Rabih Nassif, Union City, CA (US); Paramjit S. Banwait, Fremont, CA (US); David Petsko, Akron, OH (US); Travis M. Spoor, Palo Alto, CA (US); Al-Thaddeus Avestruz, Ann Arbor, MI (US)

(21) Appl. No.: 18/437,135

(22) Filed: Feb. 8, 2024

Related U.S. Application Data
(60) Provisional application No. 63/483,961, filed on Feb. 8, 2023.

(51) Int. Cl. A61N 1/378; H02J 50/10

(52) U.S. Cl. CPC A61N 1/3787; H02J 50/10

(57) ABSTRACT

Wireless power transfer devices and associated systems and methods are disclosed herein. Various embodiments of the present technology relate to devices, systems, and methods for delivering power to an implantable device, such as an implantable neuromodulation device configured to modulate a hypoglossal nerve of a patient. According to some embodiments, the present technology includes an external system comprising a control unit coupled to an external device. The external device can comprise a carrier carrying an antenna configured to conduct electrical current such that the antenna generates an electromagnetic field. When the implantable neuromodulation device is positioned within the electromagnetic field generated by the antenna, current can be induced in an antenna of the implantable neuromodulation device such that power is delivered to the implantable neuromodulation device.

### CROSS-REFERENCE TO RELATED APPLICATION(S)

The present application claims the benefit of priority to U.S. Patent Application No. 63/483,961 filed Feb. 8, 2023, which is incorporated by reference herein in its entirety.

The present application is related to the following applications, each of which is incorporated by reference herein in its entirety: International Patent Application No. PCT/US24/15058, titled WIRELESS POWER TRANSFER, filed Feb. 8, 2024, U.S. Provisional Patent Application No. 63/377,969, filed Sep. 30, 2022, titled NEUROMODULATION DEVICES AND ASSOCIATED SYSTEMS AND METHODS, U.S. patent application Ser. No. 16/865,541, filed May 4, 2020, titled IMPLANTABLE STIMULATION POWER RECEIVER, SYSTEMS, AND METHODS, U.S. patent application Ser. No. 16/866,488, filed May 4, 2020, titled SYSTEMS AND METHODS TO IMPROVE SLEEP DISORDERED BREATHING USING CLOSED-LOOP FEEDBACK, U.S. patent application Ser. No. 16/866,523, filed May 4, 2020, titled SYSTEMS AND METHODS FOR IMPROVING SLEEP DISORDERED BREATHING, and U.S. patent application Ser. No. 16/865,668, filed May 4, 2020, titled BIASED NEUROMODULATION LEAD AND METHOD OF USING SAME.

### TECHNICAL FIELD

The present technology relates to wireless power transfer devices, systems, and methods. Various embodiments of the present technology relate to devices, systems, and methods for providing power to implantable neuromodulation devices.

### BACKGROUND

Sleep disordered breathing (SDB), such as upper airway sleep disorders (UASDs), is a condition that occurs that diminishes sleep time and sleep quality, resulting in patients exhibiting symptoms that include daytime sleepiness, tiredness, and lack of concentration. Obstructive sleep apnea (OSA), the most common type of SDB, affects one in five adults in the United States. One in 15 adults has moderate to severe OSA and requires treatment. Untreated OSA results in reduced quality of life measures and increased risk of disease, including hypertension, stroke, heart disease, and others.

OSA is characterized by the complete obstruction of the airway, causing breathing to cease completely (apnea) or partially (hypopnea). During sleep, the tongue muscles relax. In this relaxed state, the tongue may lack sufficient muscle tone to prevent the tongue from changing its normal tonic shape and position. When the base of the tongue and/or soft tissue of the upper airway collapse, the upper airway channel is blocked, causing an apnea event. Blockage of the upper airway prevents air from flowing into the lungs, thereby decreasing the patient's blood oxygen level, which in turn increases blood pressure and heart dilation. This causes a reflexive forced opening of the upper airway channel until normal patency is regained, followed by normal respiration until the next apneic event. These reflexive forced openings briefly arouse the patient from sleep.

Current treatment options range from drug intervention, non-invasive approaches, to more invasive surgical procedures. In many of these instances, patient acceptance and therapy compliance are well below desired levels, rendering the current solutions ineffective as a long-term solution. Continuous positive airway pressure (CPAP), for example, is a standard treatment for OSA. While CPAP is non-invasive and highly effective, it is not well tolerated by all patients and has several side effects. Patient compliance and/or tolerance for CPAP is often reported to be between 40% and 60%. Surgical treatment options for OSA, such as anterior tongue muscle repositioning, orthognathic bimaxillary advancement, uvula-palatalpharyngoplasty, and tracheostomy are available too. However, these procedures tend to be highly invasive, irreversible, and have poor and/or inconsistent efficacy. Even the more effective surgical procedures are undesirable because they usually require multiple invasive and irreversible operations, they may alter a patient's appearance (e.g., maxillo-mandibular advancement), and/or they may be socially stigmatic (e.g., tracheostomy) and have extensive morbidity.

### SUMMARY

The subject technology is illustrated, for example, according to various aspects described below, including with reference to FIGS. 1A-26E. Various examples of aspects of the subject technology are described as numbered clauses (1, 2, 3, etc.) for convenience. These are provided as examples and do not limit the subject technology.

1. A device for use with an implant implanted in a patient at a first anatomical region, the device comprising:
a carrier configured to be positioned on a surface, the carrier having non-overlapping first and second regions; and
an antenna carried by the carrier and configured to generate a magnetic field that is denser at the first region of the carrier than the second region, wherein the magnetic field is configured to power the implant when the carrier is positioned on the surface and proximate the patient such that the first region of the carrier is aligned with the first anatomical region and the second region of the carrier is aligned with a second anatomical region of the patient, the second anatomical region having a lower soft tissue to bone ratio than the first anatomical region, and wherein, when the magnetic field is powering the implant, a specific absorption rate (SAR) parameter within the patient tissue does not exceed a predetermined threshold.

2. The device of Clause 1, wherein the first region of the carrier exhibits a greater amount of magnetic flux from the antenna than the second region of the carrier.

3. The device of Clause 1 or 2, wherein the magnetic field has a component that is configured to extend through an antenna of the implant in a direction substantially perpendicular to a radial dimension of the antenna of the implant.

4. The device of Clause 3, wherein the component of the magnetic field is substantially perpendicular to the surface.

5. The device of Clause 3 or Clause 4, wherein the component of the magnetic field is configured to extend through the antenna of the implant in the direction substantially perpendicular to the radial dimension of the antenna of the implant across a range of nod angles, axial head angles, head positions, and/or head rotations.

6. The device of any of one of Clause 3 to Clause 5, wherein an average magnitude of the component of the magnetic field is at least 2 A/m in a volume of at least 25 cubic centimeters.

7. The device of any one of the preceding Clauses, wherein the magnetic field is configured to deliver a power between about 5 mW and about 50 mW to the implant when the carrier is positioned on the surface and proximate the patient such that the first region of the carrier is aligned with the first anatomical region and the second region of the carrier is aligned with the second anatomical region of the patient.

8. The device of Clause 7, wherein the magnetic field is configured to deliver a power between about 5 mW and about 50 mW to the implant when the carrier is positioned on the surface and proximate the patient such that the first region of the carrier is aligned with the first anatomical region and the second region of the carrier is aligned with the second anatomical region of the patient, the implant being substantially ovular and having a diameter of about 2 cm to about 4 cm.

9. The device of any one of the preceding Clauses, wherein the first anatomical region comprises a head of the patient.

10. The device of any one of the preceding Clauses, wherein the second anatomical region is inferior of the head of the patient.

11. The device of any one of the preceding Clauses, wherein the second anatomical region comprises a neck and/or a back of the patient.

12. The device of any one of the preceding Clauses, wherein the second anatomical region has second electrical conductivity that is greater than a first electrical conductivity of the first anatomical region.

13. The device of any one of the preceding Clauses, wherein the second anatomical region is positioned closer to the carrier than the first anatomical region along a dimension substantially perpendicular to the surface.

14. The device of any one of the preceding Clauses, wherein the first region of the antenna comprises a first length of conductive material and the second region of the antenna comprises a second length of conductive material.

15. The device of Clause 14, wherein the first length is greater than the second length.

16. The device of Clause 14 or Clause 15, wherein the second length forms a single loop.

17. The device of any one of Clauses 14 to 16, wherein the first length forms at least a first loop and a second loop.

18. The device of Clause 17, wherein the second loop is contained within an internal area defined by first loop.

19. The device of Clause 17 or Clause 18, wherein the first loop is electrically coupled to the second loop in series.

20. The device of any one of Clauses 14 to 19, wherein the antenna comprises a transition region comprising:
a first segment comprising a first end portion of the first length and a first end portion of the second length, wherein the first segment is configured to carry RF current in a first direction; and
a second segment comprising a second end portion of the first length and a second end portion of the second length, wherein the second segment is configured to carry RF current in a second direction opposite the first direction,
wherein at least a portion of the first segment and at least a portion of the second segment overlap along a thickness dimension of the antenna in the transition region.

21. The device of Clause 20, wherein the first and second segments are positioned at an angle of less than about 30 degrees relative to each other within a plane substantially perpendicular to the thickness dimension.

22. The device of Clause 20 or Clause 21, wherein the first and second segments are aligned along the thickness dimension of the carrier.

23. The device of any one of the preceding Clauses, further comprising at least one capacitor electrically coupled to the conductive material.

24. The device of Clause 23, wherein the at least one capacitor electrically coupled to the conductive material in series.

25. The device of any one of the preceding Clauses, wherein the first region of the carrier is substantially coplanar with the second region of the carrier.

26. The device of any one of the preceding Clauses, wherein the surface is a sleeping surface that the patient lies upon during sleep.

27. The device of any one of the preceding Clauses, wherein the first region is positioned on one side of a midline of the carrier and the second region is positioned on another side of the midline of the carrier.

28. The device of Clause 27, wherein a direction of current flow through the antenna reverses at the midline.

29. The device of Clause 27 or 28, wherein the midline substantially bisects the carrier.

30. The device of any one of the preceding Clauses, wherein the antenna has a large quadrupole moment and a small dipole moment such that the antenna is configured to produce an electromagnetic field with little electromagnetic radiation at far distances from the antenna.

31. The device of any one of the preceding Clauses, wherein the SAR parameter comprises a peak spatial-average SAR averaged over any 1 gram of tissue of the patient, except at extremities of the patient, and averaged over no more than 30 minutes, and wherein the predetermined threshold comprises 1.6 W/kg.

32. The device of any one of the preceding Clauses, wherein the SAR parameter comprises a peak spatial-average SAR averaged over any 10 grams of tissue at extremities of the patient, and averaged over no more than 30 minutes, and wherein the predetermined threshold comprises 4 W/kg.

33. The device of any one of the preceding Clauses, wherein the SAR parameter comprises an average SAR averaged over an entire body of the patient and averaged over no more than 30 minutes, and wherein the predetermined threshold comprises 0.08 W/kg.

34. The device of any one of the preceding Clauses, wherein the SAR parameter comprises a peak spatial-average SAR averaged over any 1 gram of tissue, except at extremities of the patient, and averaged over no more than 6 minutes, and wherein the predetermined threshold comprises 8 W/kg.

35. The device of any one of the preceding Clauses, wherein the SAR parameter comprises a peak spatial-average SAR averaged over any 10 grams of tissue at extremities of the patient, and averaged over no more than 6 minutes, and wherein the predetermined threshold comprises 20 W/kg.

36. The device of any one of the preceding Clauses, wherein the SAR parameter comprises an average SAR averaged over an entire body of the patient and averaged over no more than 6 minutes, and wherein the predetermined threshold comprises 0.4 W/kg.

37. A device for use with an implant implanted in a patient at a first anatomical region, the device comprising:
a carrier configured to be positioned on a surface, the carrier having non-overlapping first and second regions; and
an antenna carried by the carrier and comprising a conductive material having a first length in a first configuration with one or more loops within the first region and a second length in a second configuration with one or more loops within the second region,
wherein the first and second configurations include different amounts of the conductive material,
wherein the antenna is configured to produce a magnetic field configured to power the implant when the carrier is positioned on the surface and proximate the patient such that the first region of the carrier is aligned with the first anatomical region and the second region of the carrier is aligned with a second anatomical region of the patient, and wherein, when the magnetic field is powering the implant, a specific absorption rate (SAR) parameter within the patient does not exceed a predetermined threshold.

38. The device of Clause 37, wherein the magnetic field has a component that is configured to extend through an antenna of the implant in a direction substantially perpendicular to a radial dimension of the antenna of the implant.

39. The device of Clause 38, wherein the component of the magnetic field is substantially perpendicular to the surface.

40. The device of Clause 38 or Clause 39, wherein the component of the magnetic field is configured to extend through the antenna of the implant in the direction substantially perpendicular to the radial dimension of the antenna of the implant across a range of nod angles, axial head angles, head positions, and/or head rotations.

41. The device of any one of the preceding Clauses, wherein an average magnitude of the component of the magnetic field is at least 2 A/m in a volume of at least 25 cubic centimeters.

42. The device of any one of the preceding Clauses, wherein the magnetic field is configured to deliver a power between about 5 mW and about 50 mW to the implant when the carrier is positioned on the surface and proximate the patient such that the first region of the carrier is aligned with the first anatomical region and the second region of the carrier is aligned with the second anatomical region of the patient.

43. The device of any one of the preceding Clauses, wherein the first anatomical region comprises a head of the patient.

44. The device of any one of the preceding Clauses, wherein the second anatomical region is inferior of the head of the patient.

45. The device of any one of the preceding Clauses, wherein the second anatomical region comprises a neck and/or a back of the patient.

46. The device of any one of the preceding Clauses, wherein the second anatomical region has a larger volume than the first anatomical region and/or, wherein the second anatomical region is less round than the first anatomical region.

47. The device of any one of the preceding Clauses, wherein the second anatomical region is positioned closer to the carrier than the first anatomical region along a dimension substantially perpendicular to the surface.

48. The device of any one of the preceding Clauses, wherein the first region of the antenna comprises a first density of conductive material and the second region of the antenna comprises a second density of conductive material.

49. The device of Clause 48, wherein the first density is greater than the second density.

50. The device of any one of the preceding Clauses, wherein the second length forms a single loop.

51. The device of any one of the preceding Clauses, wherein the first length forms at least a first loop and a second loop.

52. The device of Clause 51, wherein the second loop is contained within an internal area defined by first loop.

53. The device of Clause 51 or Clause 52, wherein the first loop is electrically coupled to the second loop in series.

54. The device of any of the preceding Clauses, wherein the first and second configurations differ in at least one of length, number of loops, or size of loops.

55. The device of any of the preceding Clauses, wherein the first region of the carrier exhibits a greater amount of magnetic flux from the antenna than the second region of the carrier.

56. The device of any one of any of the preceding Clauses, wherein the antenna comprises a transition region comprising:
a first segment comprising a first end portion of the first length and a first end portion of the second length, wherein the first segment is configured to carry RF current in a first direction; and
a second segment comprising a second end portion of the first length and a second end portion of the second length, wherein the second segment is configured to carry RF current in a second direction opposite the first direction,
wherein at least a portion of the first segment and at least a portion of the second segment overlap along a thickness dimension of the antenna in the transition region.

57. The device of Clause 56, wherein the first and second segments are positioned at an angle of less than about 30 degrees relative to each other within a plane substantially perpendicular to the thickness dimension.

58. The device of Clause 56 or Clause 57, wherein the first and second segments are aligned along the thickness dimension of the carrier.

59. The device of any one of the preceding Clauses, further comprising at least one capacitor electrically coupled to the conductive material.

60. The device of Clause 59, wherein the at least one capacitor electrically coupled to the conductive material in series.

61. The device of any one of the preceding Clauses, wherein the first region of the carrier is substantially coplanar with the second region of the carrier.

62. The device of any one of the preceding Clauses, wherein the surface is a sleeping surface that the patient lies upon during sleep.

63. The device of any one of the preceding Clauses, wherein the first region is positioned on one side of a midline of the carrier and the second region is positioned on another side of the midline of the carrier.

64. The device of Clause 63, wherein a direction of current flow through the antenna reverses at the midline.

65. The device of Clause 63 or 64, wherein the midline substantially bisects the carrier.

66. The device of any one of the preceding Clauses, wherein the antenna has a large quadrupole moment and a small dipole moment such that the antenna is configured to produce an electromagnetic field with little electromagnetic radiation at far distances from the antenna.

67. The device of any one of the preceding Clauses, wherein the SAR parameter comprises a peak spatial-average SAR averaged over any 1 gram of tissue of the patient, except at extremities of the patient, and averaged over no more than 30 minutes, and wherein the predetermined threshold comprises 1.6 W/kg.

68. The device of any one of the preceding Clauses, wherein the SAR parameter comprises a peak spatial-average SAR averaged over any 10 grams of tissue at extremities of the patient, and averaged over no more than 30 minutes, and wherein the predetermined threshold comprises 4 W/kg.

69. The device of any one of the preceding Clauses, wherein the SAR parameter comprises an average SAR averaged over an entire body of the patient and averaged over no more than 30 minutes, and wherein the predetermined threshold comprises 0.08 W/kg.

70. The device of any one of the preceding Clauses, wherein the SAR parameter comprises a peak spatial-average SAR averaged over any 1 gram of tissue, except at extremities of the patient, and averaged over no more than 6 minutes, and wherein the predetermined threshold comprises 8 W/kg.

71. The device of any one of the preceding Clauses, wherein the SAR parameter comprises a peak spatial-average SAR averaged over any 10 grams of tissue at extremities of the patient, and averaged over no more than 6 minutes, and wherein the predetermined threshold comprises 20 W/kg.

72. The device of any one of the preceding Clauses, wherein the SAR parameter comprises an average SAR averaged over an entire body of the patient and averaged over no more than 6 minutes, and wherein the predetermined threshold comprises 0.4 W/kg.

73. A device for use with an implant implanted in a patient at a first anatomical region, the device comprising:
a carrier configured to be positioned on a surface, the carrier having a first side and a second side opposite the first side, wherein the carrier comprises a first region between the first side and a midline and a second region between the second side and the midline, and wherein current flows through the first region in a first direction and current flows through the second region in a second direction opposite the first such that the flow of current through the antenna reverses at the midline; and
an antenna carried by the carrier and comprising conductive material forming at least two first loops at the first region of the carrier and a second loop at the second region of the carrier, wherein the antenna is configured to produce a magnetic field configured to power the implant when the carrier is positioned on the surface and proximate the patient such that the first region of the carrier is aligned with the first anatomical region and the second region of the carrier is aligned with a second anatomical region of the patient, wherein, when the magnetic field is powering the implant, a specific absorption rate (SAR) parameter within the patient does not exceed a predetermined threshold.

74. The device of Clause 73, wherein the antenna is configured to produce a magnetic field having a component substantially aligned with the width dimension of the antenna.

75. The device of Clause 74, wherein the component of the magnetic field is substantially perpendicular to the surface.

76. The device of Clause 74 or Clause 75, wherein the component of the magnetic field is configured to extend through the antenna of the implant in the direction substantially perpendicular to the radial dimension of the antenna of the implant across a range of nod angles, axial head angles, head positions, and/or head rotations.

77. The device of any one of the preceding Clauses, wherein, when the magnetic field is powering the implant, the component has an average magnitude at least 2 A/m at the implant.

78. The device of any one of the preceding Clauses, wherein, when the magnetic field is powering the implant, the component has an average magnitude at least 2 A/m over a volume of at least 25 cubic centimeters.

79. The device of any one of the preceding Clauses, wherein the at least two first loops are electrically coupled in series with the second loop.

80. The device of any one of the preceding Clauses, wherein the at least two first loops are electrically coupled in series with one another.

81. The device of any one of the preceding Clauses, wherein electrical current is configured to flow through each of the at least two first loops in a first direction and electrical current is configured to flow through the second loop in a second direction opposite the first direction.

82. The device of any one of the preceding Clauses, wherein the at least two first loops comprise a major loop and a minor loop.

83. The device of Clause 82, wherein the major loop encloses a first area and the minor loop encloses a second area less than the first area.

84. The device of any one of Clauses 81 to 83, wherein the at least two first loops comprise a major loop and at least two minor loops.

85. The device of Clause 84, wherein the at least two minor loops are spaced apart from one another along a length dimension of the carrier, the length dimension of the carrier being substantially perpendicular to the width dimension.

86. The device of Clause 84 or Clause 85, wherein the at least two minor loops enclose substantially equivalent areas.

87. The device of any of the preceding Clauses, wherein the first region of the carrier exhibits a greater amount of magnetic flux from the antenna than the second region of the carrier.

88. The device of any one of any of the preceding Clauses, wherein the first region of the antenna comprises a first length of conductive material and the second region of the antenna comprises a second length of conductive material, wherein the antenna further comprises a transition region comprising:
a first segment comprising a first end portion of the first length and a first end portion of the second length, wherein the first segment is configured to carry current in the first direction; and
a second segment comprising a second end portion of the first length and a second end portion of the second length, wherein the second segment is configured to carry current in the second direction,
wherein at least a portion of the first segment and at least a portion of the second segment overlap along a thickness dimension of the antenna in the transition region.

89. The device of Clause 88, wherein the first and second segments are positioned at an angle of less than about 30 degrees relative to each other within a plane substantially perpendicular to the thickness dimension.

90. The device of Clause 88 or Clause 89, wherein the first and second segments are aligned along the thickness dimension of the carrier.

91. The device of any one of the preceding Clauses, wherein the antenna is configured to operate at a frequency of about 6.78 MHz.

92. The device of any one of the preceding Clauses, wherein the antenna comprises a capacitor electrically coupled to the conductive material to produce a real input impedance at an operating frequency of the antenna.

93. The device of any one of the preceding Clauses, wherein the antenna comprises a capacitor electrically coupled to the conductive material in series.

94. The device of any one of the preceding Clauses, wherein the antenna comprises a capacitor electrically coupled to the conductive material in parallel.

95. The device of any one of Clauses 92 to 94, wherein the capacitor has a capacitance between about 500 pF and about 2000 pF.

96. The device of any one of Clauses 92 to 95, wherein the capacitor has a tunable capacitance.

97. The device of any one of Clauses 92 to 96, wherein the antenna comprises a plurality of capacitors electrically coupled to the conductive material in series.

98. The device of Clause 97, wherein adjacent ones of the plurality of capacitors are spaced apart along a length of the conductive material.

99. The device of Clause 97 or Clause 98, wherein the plurality of capacitors comprises between about 8 capacitors and about 15 capacitors.

100. The device of any one of Clauses 97 to 99, wherein at least two capacitors of the plurality of capacitors have different capacitances.

101. The device of any one of the preceding Clauses, wherein a maximum voltage of the antenna does not exceed 600 V.

102. The device of any one of Clauses 97 to 101, wherein a voltage across one length of conductive material between a first pair of capacitors of the plurality of is substantially equivalent to a voltage across a second pair of capacitors of the plurality of capacitors.

103. The device of any one of the preceding Clauses, wherein the first anatomical region is a head of the patient.

104. The device of any one of the preceding Clauses, wherein the second anatomical region is a neck of the patient.

105. The device of any one of the preceding Clauses, wherein the second anatomical region is a back of the patient.

106. The device of any one of the preceding Clauses, wherein the second anatomical region has a greater soft tissue to bone ratio than the first anatomical region.

107. The device of any one of the preceding Clauses, wherein the second anatomical region is positioned closer to the carrier than the first anatomical region along a dimension substantially perpendicular to the surface.

108. The device of any one of the preceding Clauses, wherein the carrier comprises a fabric.

109. The device of any one of the preceding Clauses, wherein the carrier comprises a perforated material.

110. The device of any one of the preceding Clauses, wherein the carrier comprises a foam.

111. The device of any one of the preceding Clauses, wherein the carrier comprises at least two layers.

112. The device of Clause 111, wherein the at least two layers comprise distinct materials.

113. The device of Clause 111 or Clause 112, wherein the conductive material is positioned between the at least two layers.

114. The device of any one of the preceding Clauses, wherein the carrier comprises a conductive material.

115. The device of any one of the preceding Clauses, wherein the carrier comprises a ferromagnetic material.

116. The device of any one of the preceding Clauses, wherein antenna comprises a substrate carrying the conductive material, the substrate being carried by the carrier.

117. The device of Clause 116, wherein the substrate comprises a polyimide.

118. The device of any one of the preceding Clauses, wherein the antenna has a large quadrupole moment and a small dipole moment such that the antenna is configured to produce an electromagnetic field with little electromagnetic radiation at far distances from the antenna.

119. The device of any one of the preceding Clauses, wherein the SAR parameter comprises a peak spatial-average SAR averaged over any 1 gram of tissue of the patient, except at extremities of the patient, and averaged over no more than 30 minutes, and wherein the predetermined threshold comprises 1.6 W/kg.

120. The device of any one of the preceding Clauses, wherein the SAR parameter comprises a peak spatial-average SAR averaged over any 10 grams of tissue at extremities of the patient, and averaged over no more than 30 minutes, and wherein the predetermined threshold comprises 4 W/kg.

121. The device of any one of the preceding Clauses, wherein the SAR parameter comprises an average SAR averaged over an entire body of the patient and averaged over no more than 30 minutes, and wherein the predetermined threshold comprises 0.08 W/kg.

122. The device of any one of the preceding Clauses, wherein the SAR parameter comprises a peak spatial-average SAR averaged over any 1 gram of tissue, except at extremities of the patient, and averaged over no more than 6 minutes, and wherein the predetermined threshold comprises 8 W/kg.

123. The device of any one of the preceding Clauses, wherein the SAR parameter comprises a peak spatial-average SAR averaged over any 10 grams of tissue at extremities of the patient, and averaged over no more than 6 minutes, and wherein the predetermined threshold comprises 20 W/kg.

124. The device of any one of the preceding Clauses, wherein the SAR parameter comprises an average SAR averaged over an entire body of the patient and averaged over no more than 6 minutes, and wherein the predetermined threshold comprises 0.4 W/kg.

125. A neuromodulation system comprising:
an external system comprising:
   an external device comprising any of the devices of Clauses 1-124, wherein the carrier of the external device is configured to be positioned extracorporeally between the patient and the surface on which the external device is positioned; and
   a control unit electrically coupled to the antenna of the external device, wherein the control unit is configured to deliver an RF current to the antenna such that the antenna produces the magnetic field; and
an implantable neuromodulation device configured to be implanted in the first anatomical region of the patient, the implantable neuromodulation device comprising a second antenna and a lead extending away from the second antenna and carrying an electrode, wherein, the second antenna is configured to inductively couple to the antenna of the external device when the second antenna is positioned within the magnetic field produced by the antenna of the external device such that an RF current is induced in the second antenna.

126. The system of Clause 125, wherein the implantable neuromodulation device does not include a battery.

127. The system of any one of the preceding Clauses, wherein the RF current induced in the second antenna is delivered to the electrode carried by the lead.

128. The system of any one of the preceding Clauses, wherein the implantable device is configured to deliver electrical stimulation energy from the electrode to a tissue in the first anatomical region of the patient.

129. The system of Clause 128, wherein the tissue is a hypoglossal nerve of the patient.

130. The system of Clause 128 or Clause 129, wherein the tissue is a genioglossus muscle of the patient.

131. The system of any one of the preceding Clauses, wherein the control unit comprises a variable matching circuit.

132. The system of Clause 131, wherein the variable matching circuit is configured to modify a presented impedance of the antenna of the external device.

133. The system of Clause 131 or Clause 132, wherein the variable matching circuit is configured to optimize the presented impedance of the antenna of the external device.

134. The system of any one of Clauses 131 to 133, wherein the variable matching circuit comprises a plurality of capacitors and a plurality of switches.

135. The system of any one of Clauses 131 to 134, wherein the variable matching circuit is configured to selectively activate or deactivate each of the plurality of capacitors via the plurality of switches.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1A is a fragmentary midline sagittal view of an upper airway of a human patient.

FIG. 1B is an illustration of the musculature and hypoglossal innervation of the human tongue.

FIG. 1C is a schematic superior view of a distal arborization of right and left hypoglossal nerves of a human patient. The hypoglossal nerves of FIG. 1C are shown as extending anteriorly from the bottom of the page to the top of the page (e.g., from the hyoid bone to the anterior mandible).

FIG. 2A is a schematic illustration of a neuromodulation system configured in accordance with several embodiments of the present technology.

FIG. 2B is a perspective view of a neuromodulation device configured in accordance with several embodiments of the present technology.

FIGS. 2C and 2D are top and side views, respectively, of the neuromodulation device of FIG. 2B.

FIGS. 3A-3F are various views of the neuromodulation device shown in FIGS. 2B-2D implanted in a human patient in accordance with several embodiments of the present technology.

FIG. 4A is a planar view of an external device in accordance with several embodiments of the present technology.

FIGS. 4B and 4C are expanded views of the first and second ends of the first and second lengths of the antenna of the external device of FIG. 4A.

FIG. 5 is a two-dimensional view of a magnetic field generated by the antenna of the external device of FIG. 4A in accordance with several embodiments of the present technology.

FIG. 6 is a planar view of an electric field generated by the antenna of the external device of FIG. 4A in accordance with several embodiments of the present technology.

FIGS. 7A and 7B are side and top views, respectively, of a patient. FIGS. 7A and 7B illustrate a specific absorption rate at various locations in the patient when the patient is positioned within an electromagnetic field generated by the antenna of the external device of FIG. 4A in accordance with several embodiments of the present technology.

FIG. 8A illustrates representative positions of an antenna of an implantable neurostimulation device implanted at a treatment site within submental and sublingual regions of a patient's head while the patient sleeps.

FIGS. 8B-8D illustrate example nod angles, axial head angles, and head rotation angles, respectively, of a patient.

FIG. 9 is a contour plot of peak spatial specific absorption rate (psSAR)-limited average magnitude of the component of the magnetic field perpendicularly penetrating the implantable device antenna over a variety of positions of a patient's chin relative to the antenna of the external device.

FIG. 10 is a planar view of an external device in accordance with several embodiments of the present technology.

FIG. 11 is a planar view of an electric field generated by the antenna of the external device of FIG. 10 in accordance with several embodiments of the present technology.

FIG. 12 is a planar view of an external device in accordance with several embodiments of the present technology.

FIG. 13 is a two-dimensional view of a magnetic field generated by the antenna of the external device of FIG. 12 in accordance with several embodiments of the present technology.

FIGS. 14A and 14B are side and top views, respectively, of a patient. FIGS. 14A and 14B illustrate a specific absorption rate at various locations in the patient when the patient is positioned within an electromagnetic field generated by the antenna of the external device of FIG. 12 in accordance with several embodiments of the present technology.

FIG. 15 is a planar view of an external device in accordance with several embodiments of the present technology.

FIG. 16 is a two-dimensional view of a magnetic field generated by the antenna of the external device of FIG. 15 in accordance with several embodiments of the present technology.

FIGS. 17A and 17B are side and top views, respectively, of a patient. FIGS. 17A and 17B illustrate a specific absorption rate at various locations in the patient when the patient is positioned within an electromagnetic field generated by the antenna of the external device of FIG. 15 in accordance with several embodiments of the present technology.

FIGS. 18-22 are planar views of external devices in accordance with several embodiments of the present technology.

FIG. 23 is a block diagram of a control unit and a second antenna of an external system in accordance with several embodiments of the present technology.

FIGS. 24A and 24B are block diagrams of control units and second antennas of external systems in accordance with several embodiments of the present technology.

FIG. 25A schematically depicts an example series matching circuit in accordance with several embodiments of the present technology.

FIG. 25B schematically depicts an example series-shunt matching circuit in accordance with several embodiments of the present technology.

FIGS. 26A and 26B are perspective views of an external device in accordance with several embodiments of the present technology, FIG. 26C is a cutaway perspective view of the external device, FIG. 26D is a side cross-sectional view of the external device, and FIG. 26E is a planar view of an antenna of the external device.

### DETAILED DESCRIPTION

The present disclosure relates to devices, systems, and methods for wirelessly powering implantable medical devices. For example, an external system of the present technology can comprise a control unit coupled to an external device comprising a carrier carrying an antenna configured to conduct an RF current such that the antenna generates an electromagnetic field. When the implantable device is positioned within the electromagnetic field generated by the antenna, RF current can be induced in an antenna of the implantable device that can be used to power one or more electronic components carried by the implantable device. In some embodiments, the external devices and systems disclosed herein are used to power a neuromodulation system, which can be used to provide a variety of electrical therapies, including neuromodulation therapies such as nerve and/or muscle stimulation. Stimulation can induce excitatory or inhibitory neural or muscular activity. Such therapies can be used at various suitable sites within a patient's anatomy. According to some embodiments, the neuromodulation systems of the present technology are configured to treat sleep disordered breathing (SDB), including obstructive sleep apnea (OSA) and/or mixed sleep apnea, via neuromodulation of the hypoglossal nerve (HGN).

For the purpose of contextualizing the structure and operation of the neuromodulation systems and devices disclosed herein, some of the relevant anatomy and physiology are first described below. The headings provided herein are for convenience only and do not interpret the scope or meaning of the claimed present technology. Embodiments under any one heading may be used in conjunction with embodiments under any other heading. For example, any of the neuromodulation systems and devices described in connection with Sections II and III can be used with any of the external systems and external devices described in connection with Section IV.

### I. Anatomy and Physiology

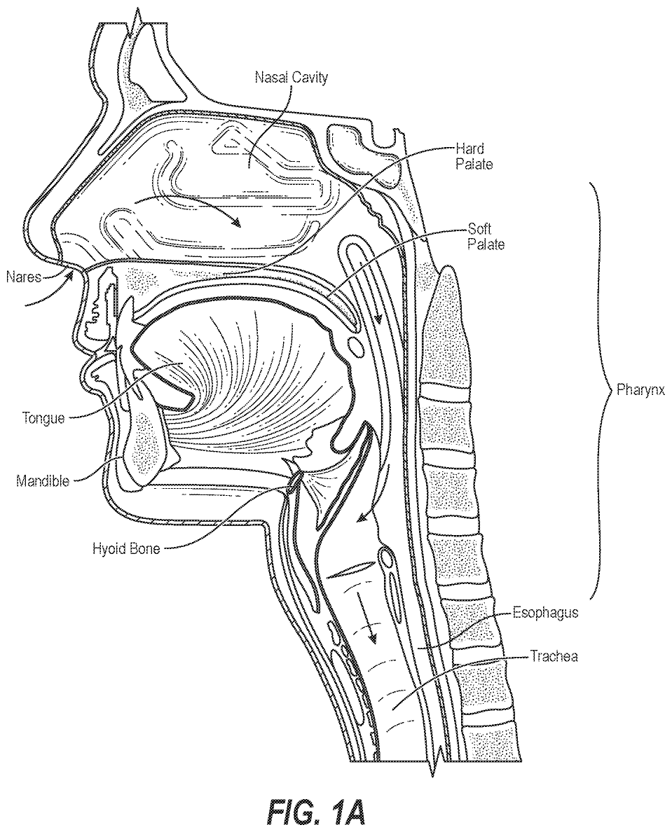

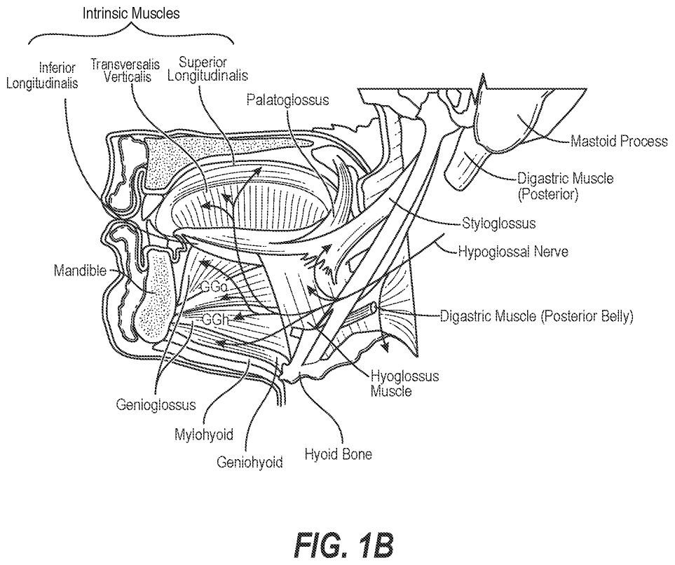

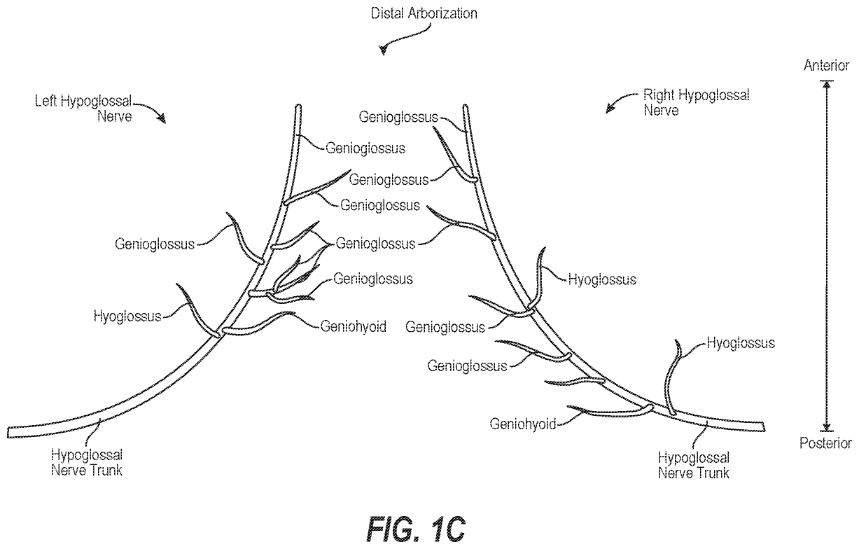

As previously mentioned, respiration in patients with SDB is frustrated due to obstruction, narrowing, and/or collapse of the upper airway during sleep. As shown in FIG. 1A, the upper airway comprises the nasal cavity, the oral cavity, the pharynx, and the larynx. Patency of the upper airway and resistance to airflow in the upper airway are controlled by a complex network of muscles under both voluntary and involuntary neuromuscular control. For example, the muscles of the tongue, the suprahyoid muscles (e.g., the geniohyoid, mylohyoid, stylohyoid, hyoglossus, and the anterior belly of the digastric muscle), and the muscles comprising the soft palate (e.g., palatal muscles) open, widen, and/or stabilize the upper airway during inspiration to counteract the negative airway pressure responsible for drawing air into the airway and the lungs.

With reference to FIG. 1B, the tongue comprises both intrinsic and extrinsic lingual muscles. Generally, activation of the intrinsic muscles changes the shape of the tongue while activation of the extrinsic muscles tends to move the position of the whole tongue. The extrinsic muscles originate at a bony attachment and insert within the tongue. They comprise the genioglossus muscle, the styloglossus muscle, the hyoglossus muscle, and the palatoglossus muscle. The intrinsic muscles both originate and insert within the tongue, and comprise the superior longitudinalis, the inferior longitudinalis, the transversalis, and the verticalis. In a patient who is awake, the brain supplies neural drive to these muscles through the HGN to maintain tongue shape and position, preventing the tongue from blocking the airway.

The lingual muscles are also functionally categorized as either retrusor or protrusor muscles and both intrinsic and extrinsic muscles fall into these categories. The retrusor muscles include the intrinsic superior and inferior longitudinalis muscles and the extrinsic hyoglossus and styloglossus muscles. The protrusor muscles include the intrinsic verticalis and transversalis muscles and the extrinsic genioglossus muscle. Contraction of the styloglossus muscle causes elevation of the tongue while depression of the tongue is the result of downward movements of hyoglossus and genioglossus muscles. Also labeled in FIG. 1B is the geniohyoid muscle, which is a suprahyoid muscle (not a tongue muscle) but still an important protrusor and pharyngeal dilator, and thus contributes to maintaining upper airway patency. It is believed that effective treatment of OSA requires stimulation of the protrusor muscles with minimal or no activation of the retrusor muscles. Thus, for neuromodulation therapy to be effective it is considered beneficial to localize stimulation to the protrusor muscles while avoiding activation of the retrusor muscles.

The largest of the tongue muscles, the genioglossus, comprises two morphological and functional compartments according to fiber distribution, action, and nerve supply. The first, the oblique compartment (GGo), comprises vertical fibers that, when contracted, depress the tongue without substantially affecting pharyngeal patency. The second, the horizontal compartment (GGh), contains longitudinal fibers that, when activated, protrude the posterior part of the tongue and enlarge the pharyngeal opening. The GGo contains Type II muscle fibers that are quickly fatigued, whereas the GGh contains Type I muscle fibers that are slower to fatigue. Accordingly, it can be advantageous to stimulate the GGh with little or no stimulation of the GGo to effectively protrude the tongue while preventing or limiting fatigue of the tongue.

The suprahyoid muscles, which comprise the mylohyoid, the geniohyoid, the stylohyoid, and the digastric (only a portion of which is shown in FIG. 1B), extend between the mandible and the hyoid bone to form the floor of the mouth. The geniohyoid is situated inferior to the genioglossus muscle of the tongue and the mylohyoid is situated inferior to the geniohyoid. Contraction of the geniohyoid and tone of the sternohyoid (an infrahyoid muscle, not shown) cooperate to pull the hyoid bone anteriorly to open and/or widen the pharyngeal lumen and stabilize the anterior wall of the hypopharyngeal region. In contrast to the genioglossus and geniohyoid, which are considered tongue protrusors, the hyoglossus and styloglossus are considered tongue retrusors. Activation of the hyoglossus and styloglossus tends to retract the tongue posteriorly, which reduces the size of the pharyngeal opening, increases airway resistance, and frustrates respiration.

As previously mentioned, all of the extrinsic and intrinsic muscles of the tongue are innervated by the HGN, with the exception of the palatoglossus, which is innervated by the vagal nerve. There are two hypoglossal nerves in the body, one on the right side of the head and one on the left side. Each hypoglossal nerve originates at a hypoglossal nucleus in the medulla oblongata of the brainstem, exits the cranium via the hypoglossal canal, and passes inferiorly through the retrostyloid space (a portion of the lateral pharyngeal space) to the occipital artery. The hypoglossal nerve then curves and courses anteriorly to the muscles of the tongue, passing between the anterior edge of the hyoglossus muscle and the posterior edge of the mylohyoid muscle into the sublingual area where it splits into a distal arborization.

FIG. 1C is a schematic superior view of the distal arborization of the right and left hypoglossal nerves. Referring to FIGS. 1B and 1C together, the HGN comprises (1) portions of the distal arborization that innervate the styloglossus and the hyoglossus (tongue retrusor muscles) and (2) portions of the distal arborization that innervate the intrinsic muscles of the tongue, the genioglossus, and the geniohyoid (tongue protrusor muscles). Additionally, the portions of the distal arborization that innervate the tongue retrusor muscles tend to be located posterior of the portions of the distal arborization that innervate the tongue protrusor muscles.

A reduction in activity of the muscles responsible for airway maintenance can result in an increase in airway resistance and a myriad of downstream effects on a patient's respiration and health. Activity of the genioglossus muscle, for example, can decrease during sleep which, whether alone or in combination with other factors (e.g., airway length, airway diameter, soft tissue volume, premature wakening, etc.), can result in substantial airway resistance and/or airway collapse leading to sleep disordered breathing, such as OSA. It is believed that in order for neuromodulation therapy to be effective, it may be beneficial to largely confine stimulation of the HGN to the portions of the distal arborization that innervate protrusor muscles while avoiding or limiting stimulation of the portions of the distal arborization that activate the retrusor muscles.

### II. Neuromodulation Systems

Various embodiments of the present technology are directed to devices, systems, and methods for modulating neurological activity and/or control of one or more nerves associated with one or more muscles involved in airway maintenance. Such neuromodulation can increase activity in targeted muscles, for example the genioglossus and geniohyoid, to reduce a patient's airway resistance and improve the patient's respiration. Moreover, targeted modulation of specific portions of the distal arborization of the hypoglossal nerve can increase activity in tongue protrusor muscles without substantially increasing activity in tongue retrusor muscles to provide a highly efficacious treatment. Additionally or alternatively, targeted modulation of specific portions of the distal arborization of the hypoglossal nerve that innervate the GGh but not portions of the distal arborization of the hypoglossal nerve that innervate the GGo can be used to effectively protrude the tongue while preventing or limiting fatigue of the tongue.

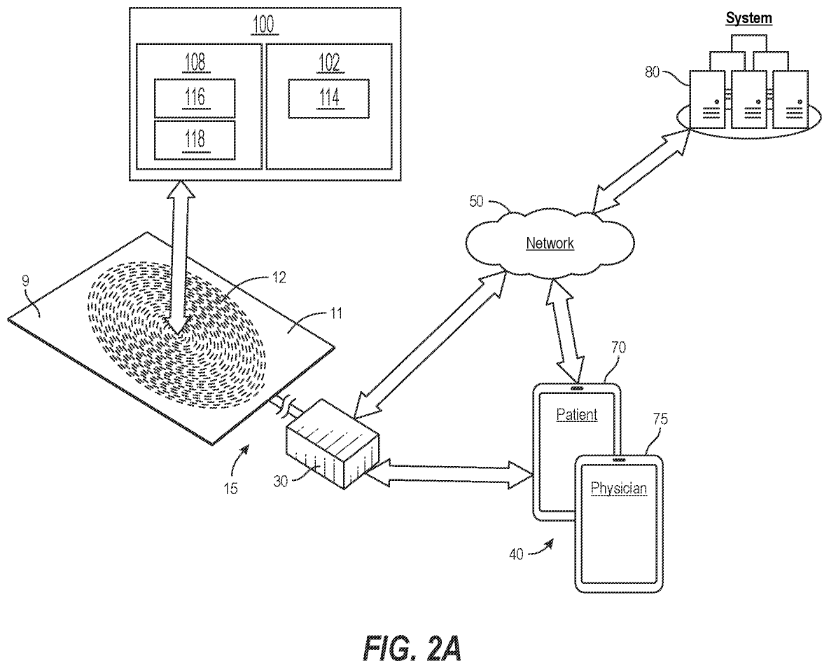

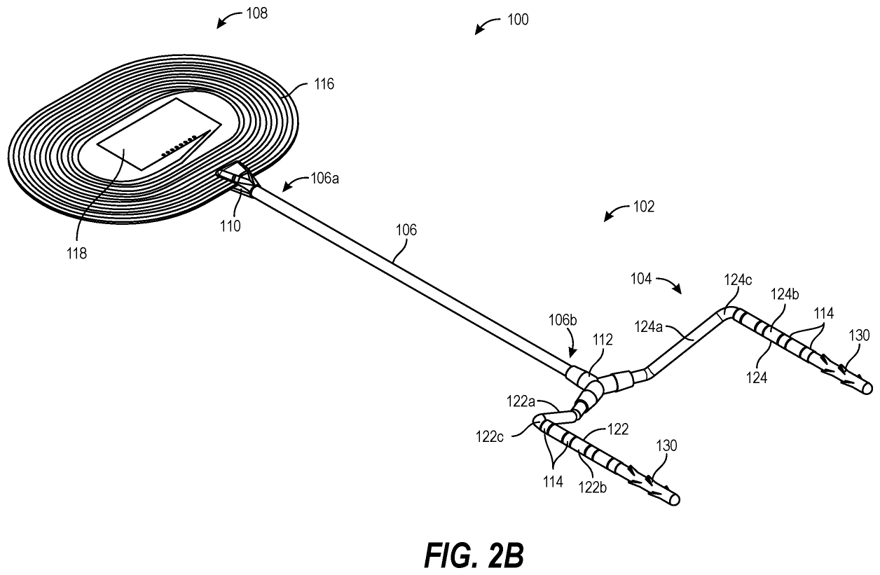

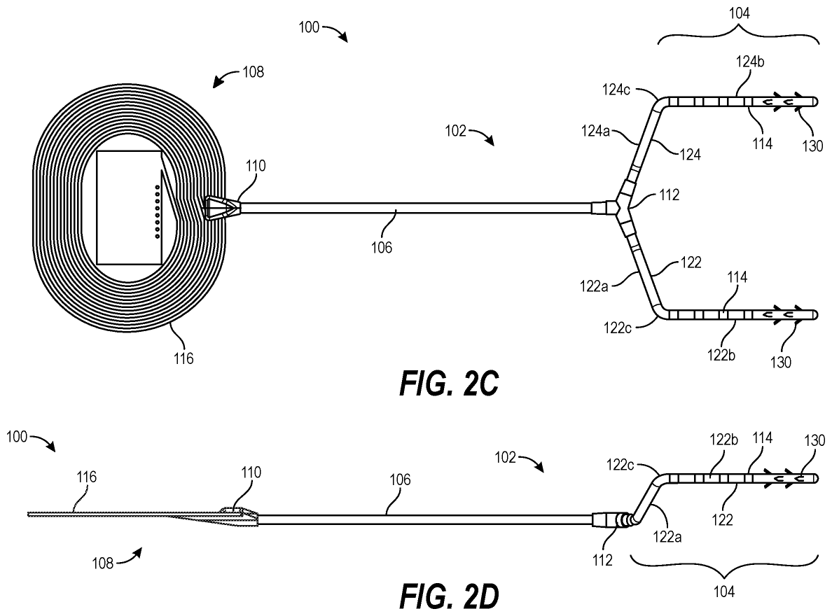

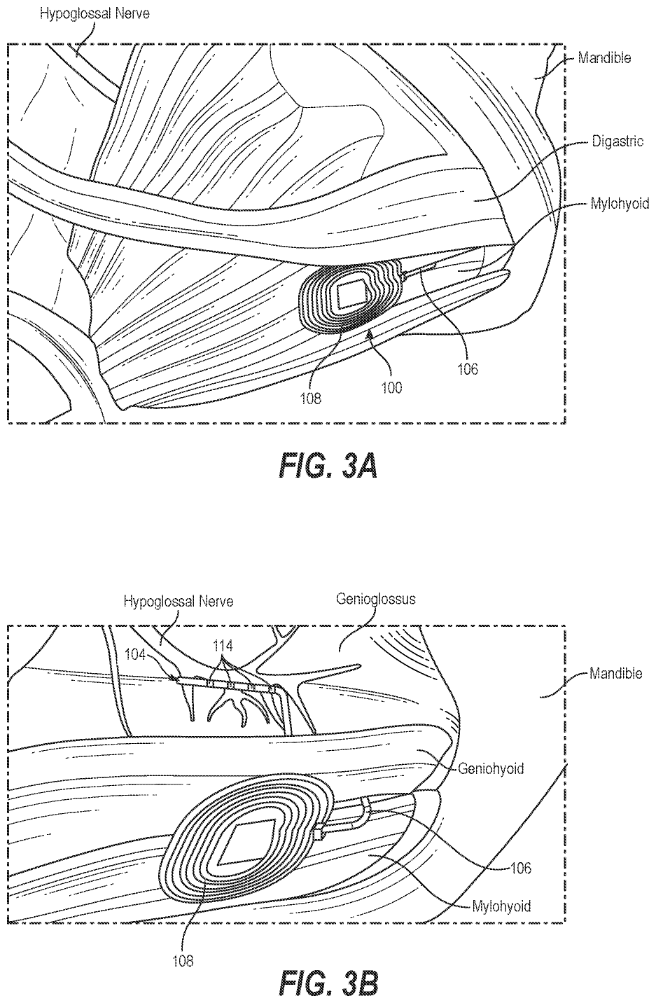

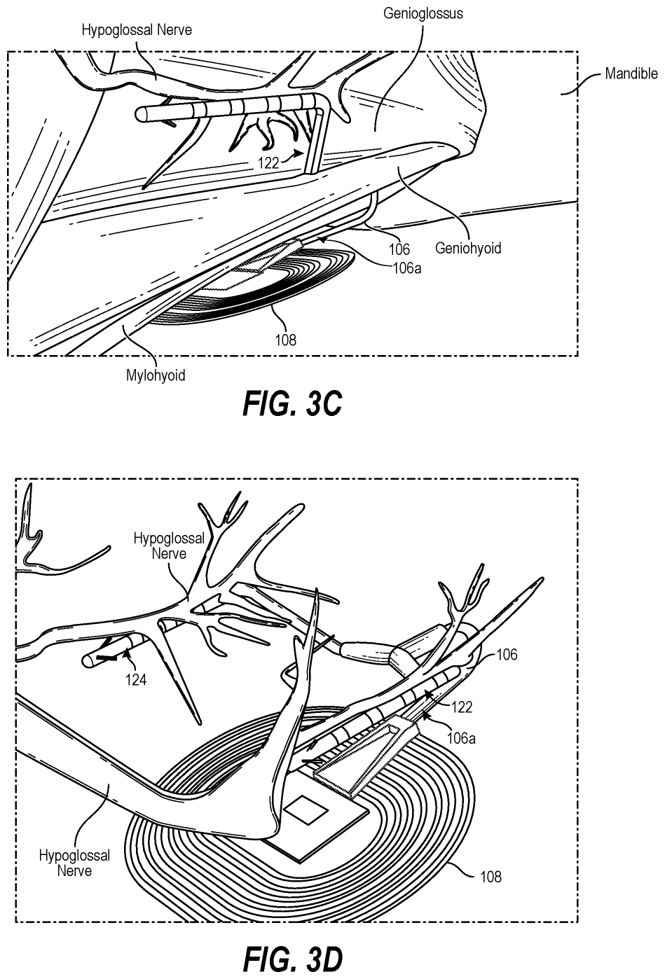

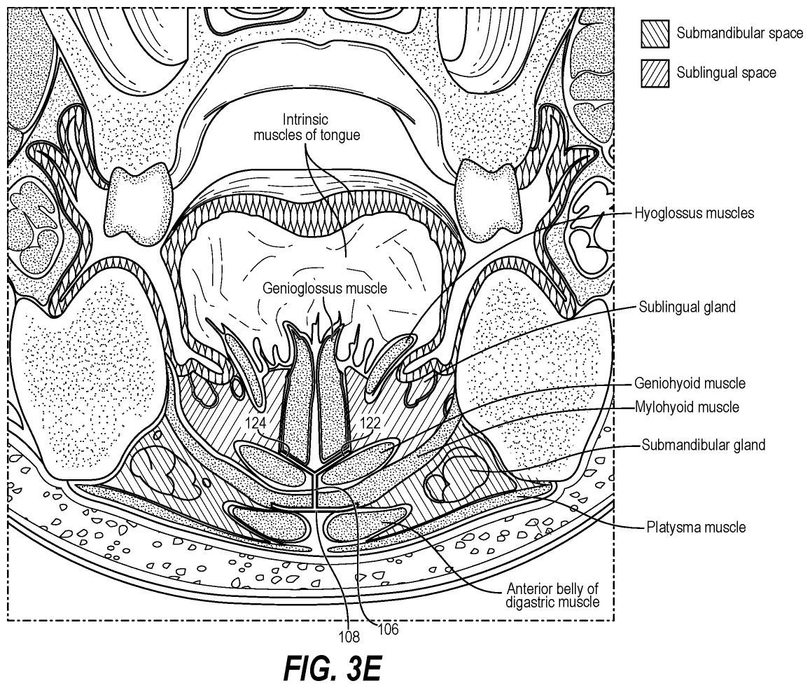

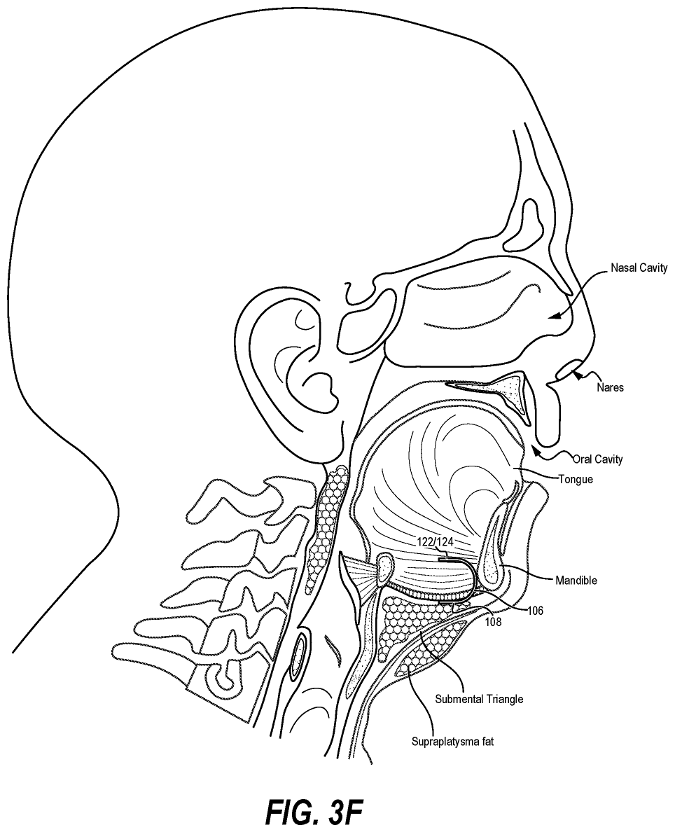

FIG. 2A shows a neuromodulation system 10 for treating SDB configured in accordance with the present technology. The system 10 can include an implantable neuromodulation device 100 and an external system 15 configured to wirelessly couple to the neuromodulation device 100. The neuromodulation device 100 can include a lead 102 having a plurality of conductive elements 114 and an electronics package 108 having a first antenna 116 and an electronics component 118. The neuromodulation device 100 is configured to be implanted at a treatment site comprising submental and sublingual regions of a patient's head, as detailed below with reference to FIGS. 3A-3F.

In use, the electronics package 108 or one or more elements thereof can be configured provide a stimulation energy to the conductive elements 114 that has a pulse width, amplitude, duration, frequency, duty cycle, and/or polarity such that the conductive elements 114 apply an electric field at the treatment site that modulates the hypoglossal nerve. The stimulation energy can be delivered according to a periodic waveform including, for example, a charge-balanced square wave comprising alternating anodic and cathodic pulses.

One or more pulses of the stimulation energy can have a pulse width between about 10 μs and about 1000 μs, between about 50 μs and about 950 μs, between about 100 μs and about 900 μs, between about 150 μs and about 800 μs, between about 200 μs and about 850 μs, between about 250 μs and about 800 μs, between about 300 μs and about 750 μs, between about 350 μs and about 700 μs, between about 400 μs and about 650 μs, between about 450 μs and about 600 μs, between about 500 μs and about 550 μs, about 50 μs, about 100 μs, about 150 μs, about 200 μs, about 250 μs, about 300 μs, about 350 μs, about 400 μs, about 450 μs, about 500 μs, about 550 μs, about 600 μs, about 650 μs, about 700 μs, about 750 μs, about 800 μs, about 850 μs, about 900 μs, about 950 μs, and/or about 1000 μs. In some embodiments, one or more pulses of the stimulation energy has a pulse width of between about 50 μs and about 450 μs.

One or more pulses of the stimulation energy can have an amplitude sufficient to cause an increase in phasic activity of a desired muscle. For example, one or more pulses of the stimulation energy can have a current-controlled amplitude between about 0.1 mA and about 5 mA. In some embodiments, the stimulation energy has an amplitude of about 0.3 mA, about 0.4 mA, about 0.5 mA, about 0.6 mA, about 0.7 mA, about 0.8 mA, about 0.9 mA, about 1 mA, about 1.5 mA, about 2 mA, about 2.5 mA, about 3 mA, about 3.5 mA, about 4 mA, about 4.5 mA, and/or about 5 mA. Additionally or alternatively, an amplitude of one or more pulses of the stimulation energy can be voltage-controlled. An amplitude of one or more pulses of the stimulation energy can be based at least in part on a size and/or configuration of the conductive elements 114, a location of the conductive elements 114 in the patient, etc.

A frequency of the pulses of the stimulation energy can be between about 10 Hz and about 50 Hz, between about 20 Hz and about 40 Hz, about 10 Hz, about 15 Hz, about 20 Hz, about 25 Hz, about 30 Hz, about 35 Hz, about 40 Hz, about 45 Hz, and/or about 50 Hz. In some embodiments, the frequency can be based on a desired effect of the stimulation energy on one or more muscles or nerves. For example, lower frequencies may induce a muscular twitch whereas higher frequencies may include complete contraction of a muscle.

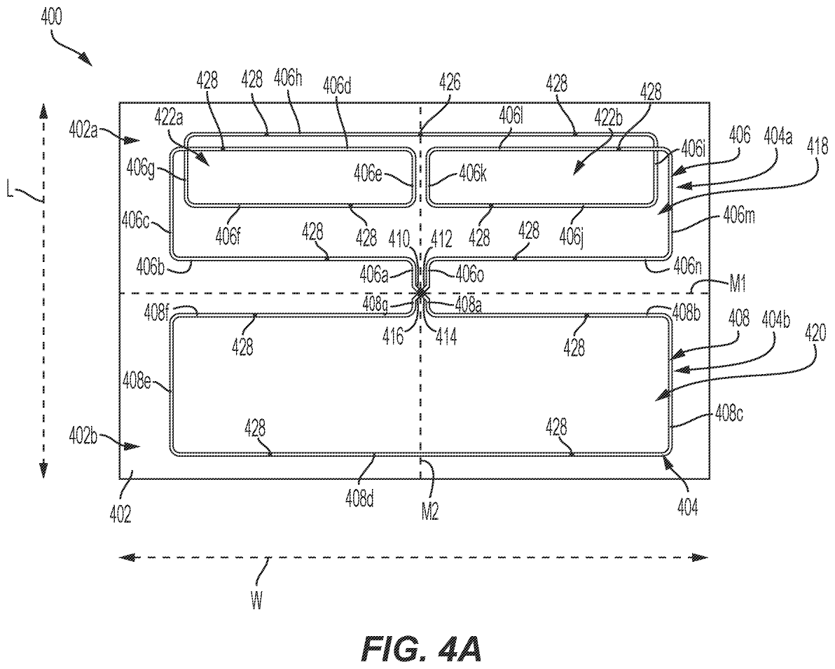

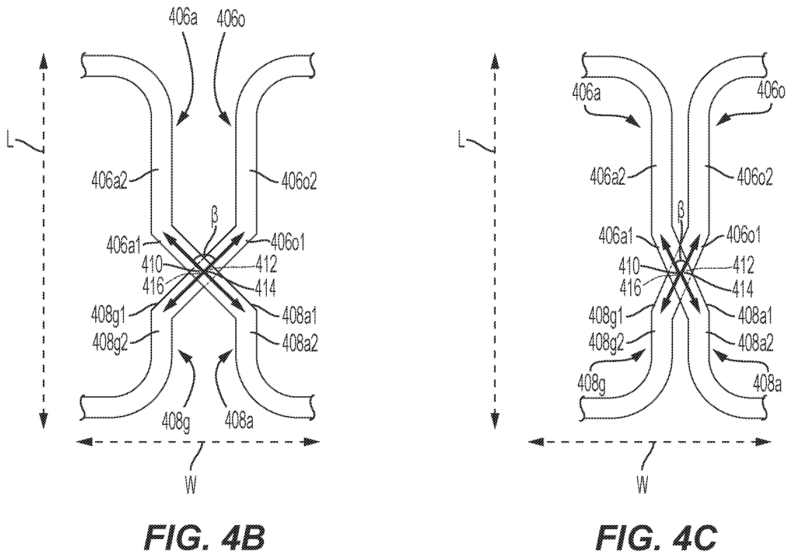

The external system 15 can comprise an external device 11 and a control unit 30 communicatively coupled to the external device 11. In some embodiments, the external device 11 is configured to be positioned proximate a patient's head while they sleep. The external device 11 can comprise a carrier 9 integrated with a second antenna 12. Additional details regarding the external system 15 and the external device 11 are provided below with reference to FIGS. 4-26E. While the control unit 30 is shown separate from the external device 11 in FIG. 2A, in some embodiments the control unit 30 can be integrated with and/or comprise a portion of the external device 11. The second antenna 12 can be configured for multiple purposes. For example, the second antenna 12 can be configured to power the neuromodulation device 100 through electromagnetic resonance. Electrical current can be induced in the first antenna 116 when it is positioned above the second antenna 12 of the external device 11, in an electromagnetic field produced by second antenna 12. The first and second antennas 116, 12 can also be configured transmit data to and/or receive data from one another via one or more wireless communication techniques (e.g., Bluetooth, WiFi, USB, etc.) to facilitate communication between the neuromodulation device 100 and the external system 15. This communication can, for example, include programming, e.g., uploading software/firmware revisions to the neuromodulation device 100, changing/adjusting stimulation settings and/or parameters, and/or adjusting parameters of control algorithms.

The control unit 30 of the external system 15 can include a processor and/or a memory that stores instructions (e.g., in the form of software, code or program instructions executable by the processor or controller) for causing the external device to generate an electromagnetic field according to certain parameters provided by the instructions. The external system 15 or one or more portions thereof, such as the control unit 30, can include and/or be configured to be coupled to a power source such as a direct current (DC) power supply, an alternating current (AC) power supply, and/or a power supply switchable between DC and AC. The processor can be used to control various parameters of the energy output by the power source, such as intensity, amplitude, duration, frequency, duty cycle, and polarity. Instead of or in addition to a processor, the external system can include drive circuitry. In such embodiments, the external system 15 or one or more portions thereof (e.g., control unit 30), can include hardwired circuit elements to provide the desired waveform delivery rather than a software-based generator. The drive circuitry can include, for example, analog circuit elements (e.g., resistors, diodes, switches, etc.) that are configured to cause the power source to supply energy to the second antenna 12 to produce an electromagnetic field according to the desired parameters. In some embodiments, the neuromodulation device 100 can be configured for communication with the external system via resonant inductive coupling.

The system 10 can also include a user interface 40 in the form of a patient device 70 and/or a physician device 75. The user interface(s) 40 can be configured to transmit and/or receive data with the external system 15, the second antenna 12, the control unit 30, the neuromodulation device 100, and/or the remote computing device(s) 80 via wired and/or wireless communication techniques (e.g., Bluetooth, WiFi, USB, etc.). In the example configuration of FIG. 2A, both the patient device 70 and physician device 75 are smartphones. The type of device could, however, vary. One or both of the patient device 70 and physician device 75 can have an application or “app” installed thereon that is user specific, e.g., a patient app or a physician app, respectively. The patient app can allow the patient to execute certain commands necessary for controlling operation of neuromodulation device 100, such as, for example, start/stop therapy, increase/decrease stimulation power or intensity, and/or select a stimulation program. In addition to the controls afforded the patient, the physician app can allow the physician to modify stimulation settings, such as pulse settings (patterns, duration, waveforms, etc.), stimulation frequency, amplitude settings, and electrode configurations, closed loop and open loop control settings and tuning parameters for the embedded software that controls therapy delivery during use.

The patient and/or physician devices 70, 75 can be configured to communicate with the other components of the system 10 via a network 50. The network 50 can be or include one or more communications networks, such as any of the following: a wired network, a wireless network, a metropolitan area network (MAN), a local area network (LAN), a wide area network (WAN), a virtual local area network (VLAN), an internet, an extranet, an intranet, and/or any other suitable type of network or combinations thereof. The patient and/or physician devices 70, 75 can be configured to communicate with one or more remote computing devices 80 via the network 50 to enable the transfer of data between the devices 70, 75 and the remote computing device(s) 80. Additionally, the external system 15 can be configured to communicate with the other components of the system 10 via the network 50. This can also enable the transfer of data between the external system 15 and remote computing device(s) 80.

The external system 15 can receive the programming, software/firmware, and settings/parameters through any of the communication paths described above, e.g., from the user interface(s) 40 directly (wired or wirelessly) and/or through the network 50. The communication paths can also be used to download data from the neuromodulation device 100, such as measured data regarding completed stimulation therapy sessions, to the external system 15. The external system 15 can transmit the downloaded data to the user interface 40, which can send/upload the data to the remote computing device(s) 80 via the network 50.

In addition to facilitating local control of the system 10, e.g., the external system 15 and the neuromodulation device 100, the various communication paths shown in FIG. 2A can also enable:
Distributing from the remoting computing device(s) 80 software/firmware updates for the patient device 70, physician device 75, external system 15, and/or neuromodulation device 100.
Downloading from the remote computing device(s) 80 therapy settings/parameters to be implemented by the patient device 70, physician device 75, external system 15, and/or neuromodulation device 100.
Facilitating therapy setting/parameter adjustments/algorithm adjustments by a remotely located physician.
Uploading data recorded during therapy sessions.
Maintaining coherency in the settings/parameters by distributing changes and adjustments throughout the system components.

The therapeutic approach implemented with the system 10 can involve implanting only the neuromodulation device 100 and leaving the external system 15 as an external component to be used only during the application of therapy. To facilitate this, the neuromodulation device 100 can be configured to be powered by the external system 15 through electromagnetic induction. In use, the second antenna 12, operated by control unit 30, can be positioned external to the patient in the vicinity of the neuromodulation device 100 such that the second antenna 12 is close to the first antenna 116 of the neuromodulation device 100. In some embodiments, the second antenna 12 is carried by a flexible carrier 9 that is configured to be positioned on, under, within, or sufficiently near a surface proximate the patient to maintain the position of the first antenna 116 within the target volume of the electromagnetic field generated by the second antenna 12. The surface can be a surface upon which the patient lies (e.g., a sleeping surface upon which the patient lies while the patient sleeps, an operating room table upon which the patient lies while the neuromodulation device 100 is being implanted, a clinic table upon which the patient lies during titration of the neuromodulation device 100, etc.). In some embodiments, the surface is a surface against which the patient reclines (e.g., a mattress with an elevated headrest, a reclining chair, etc.). According to various embodiments, the surface can be a vertical surface that the patient is positioned proximate to while the patient sits upright and/or stands upright. Through this approach, the system 10 can deliver therapy to improve SDB (such as OSA), for example, by stimulating the HGN through a shorter, less invasive procedure. The elimination of an on-board, implanted power source in favor of an inductive power scheme can eliminate the need for batteries and the associated battery changes over the patient's life.

In some embodiments, the system 10 can include one or more sensors (not shown), which may be implanted and/or external. For example, the system 10 can include one or more sensors carried by (and implanted with) the neuromodulation device 100. Such sensors can be disposed at any location along the lead 102 and/or electronics package 108. In some embodiments, one, some, or all of the conductive elements 114 can be used for both sensing and stimulation. Use of a single structure or element as the sensor and the stimulating electrode reduces the invasive nature of the surgical procedure associated with implanting the system, while also reducing the number of foreign bodies introduced into a patient. In certain embodiments, at least one of the conductive elements 114 is dedicated to sensing only.

In addition to or instead of inclusion of one or more sensors on the neuromodulation device 100, the system 10 can include one or more sensors separate from the neuromodulation device 100. In some embodiments, one or more of such sensors are wired to the neuromodulation device 100 but implanted at a different location than the neuromodulation device 100. In some embodiments, the system 10 includes one or more sensors that are configured to be wirelessly coupled to the neuromodulation device 100 and/or an external computing device (e.g., control unit 30, user interface 40, etc.). Such sensors can be implanted at the same or different location as the neuromodulation device 100, or may be disposed on the patient's skin.

The one or more sensors can be configured to record and/or detect physiological data (e.g., data originating from the patient's body) over time including changes therein. The physiological data can be used to select certain stimulation parameters and/or adjust one or more stimulation parameters during therapy. Physiological data can include an electromyography (EMG) signal, temperature, movement, body position, electroencephalography (EEG), air flow, audio data, heart rate, pulse oximetry, eye motion, and/or combinations thereof. In some embodiments, the physiological data can be used to detect and/or anticipate other physiological parameters. For example, the one or more sensors can be configured to sense an EMG signal which can be used to detect and/or anticipate physiological events such as phasic contraction of anterior lingual musculature (such as phasic genioglossus muscle contraction) and measure physiological data such as underlying tonic activity of anterior lingual musculature (such as tonic activity of the genioglossus muscle). Phasic contraction of the genioglossus muscle can be indicative of inspiration, particularly the phasic activity that is layered within the underlying tonic tone of the genioglossus muscle. Changes in physiological data include changes in one or more parameters of a measured signal (e.g., frequency, amplitude, spike rate, etc.), start and end of phasic contraction of anterior lingual musculature (such as phasic genioglossus muscle contraction), changes in underlying tonic activity of anterior lingual musculature (such as changes in tonic activity of the genioglossus muscle), and combinations thereof. In particular, changes in phasic activity of the genioglossus muscle can indicate a respiration or inspiration change and can be used to trigger stimulation. Such physiological data and changes therein can be identified in signals recorded from sensors during different phases of respiration including inspiration. As such, the one or more sensors can include EMG sensors. The one or more sensors can also include, for example, wireless or tethered sensors that measure, body temperature, movement (e.g., an accelerometer), breath sounds (e.g., audio sensors), heart rate, pulse oximetry, eye motion, etc.

In operation, the physiological data provided by the one or more sensors enables closed-loop operation of the neuromodulation device 100. For example, the sensed EMG responses from the genioglossus muscle can enable closed-loop operation of the neuromodulation device 100 while eliminating the need for a chest lead to sense respiration. Operating in closed-loop, the neuromodulation device 100 can maintain stimulation synchronized with respiration, for example, while preserving the ability to detect and account for momentary obstruction. The neuromodulation device 100 can also detect and respond to snoring, for example.

The system 10 can be configured to provide open-loop control and/or closed-loop stimulation to configure parameters for stimulation. In other words, with respect to closed-loop stimulation, the system 10 can be configured to track the patient's respiration (such as each breath of the patient) and stimulation can be applied during or prior to onset of inspiration, for example. However, with respect to open-loop stimulation, stimulation can be applying without tracking specific physiological data, such as respiration or inspiration. However, even under such an “open loop” scenario, the system 10 can still adjust stimulation and record data, to act on such information. For example, one way the system 10 can act upon such information is that the system 10 can configure parameters for stimulation to apply stimulation in an open loop fashion but can monitor the patient's respiration to know when to revert to applying stimulation on a breath to breath, close-loop fashion such that the system 10 is always working in a closed-loop algorithm to assess data. Treatment parameters of the system may be automatically adjusted in response to the physiological data. The physiological data can be stored over time and examined to change the treatment parameters; for example, the treatment data can be examined in real time to make a real time change to the treatment parameters. In some embodiments, the treatment parameters can be learned from the physiological data stored over time and used to adjust the therapy in real time. This learning can be patient-specific and/or across multiple patients.

Operating in real-time, the neuromodulation device 100 can record data (e.g., via one or more sensors) related to the stimulation session including, for example, stimulation settings, EMG responses, respiration, sleep state including different stages of REM and non-REM sleep, etc. For example, changes in phasic and tonic EMG activity of the genioglossus muscle during inspiration can serve as a trigger for stimulation or changes in stimulation can be made based on changes in phasic and tonic EMG activity of the genioglossus muscle during inspiration or during different sleep states. This recorded data can be uploaded to the user interface 40 and to the remote computing device(s) 80. Also, the patient can be queried to use the interface 40 to log data regarding their perceived quality of sleep, which can also be uploaded to the remote computing device(s) 80. Offline, the remote computing device(s) 80 can execute a software application to evaluate the recorded data to determine whether settings and control parameters can be adjusted to further optimize the stimulation therapy. The software application can, for example, include artificial intelligence (AI) models that learn from recorded therapy sessions how certain adjustments affect the therapeutic outcome for the patient. In this manner, through AI learning, the model can provide patient-specific optimized therapy.

### III. Neuromodulation Devices

FIGS. 2B-2D illustrate various views of an example configuration of the neuromodulation device 100. While specific features of the neuromodulation device 100 are discussed with reference to FIGS. 2B-2D, other configurations of the neuromodulation device 100 are possible. Example configurations of neuromodulation devices 100 within the scope of the present technology include the neuromodulation devices found in U.S. patent application Ser. No. 18/475,818, filed Sep. 27, 2023, U.S. Provisional Patent Application No. 63/377,969, filed Sep. 30, 2022, U.S. patent application Ser. No. 16/865,541, filed May 4, 2020, U.S. patent application Ser. No. 16/866,488, filed May 4, 2020, U.S. patent application Ser. No. 16/866,523, filed May 4, 2020, and U.S. patent application Ser. No. 16/865,668, filed May 4, 2020. As previously mentioned, the device 100 can be configured to be implanted at a treatment site within submental and sublingual regions of the patient's head and deliver electrical energy at the treatment site to stimulate the HGN and/or one or more tongue protruser muscles (e.g., the genioglossus, the geniohyoid, etc.). The device 100 can include an electronics package 108 and a lead 102 coupled to and extending away from the electronics package 108. The lead 102 can comprise a lead body 104 having a plurality of conductive elements 114 and an extension portion 106 extending between the lead body 104 and the electronics package 108. The extension portion 106 can have a proximal end portion 106a coupled to the electronics package 108 via a first connector 110 and a distal end portion 106b coupled to the lead body 104 via a second connector 112.

The electronics package 108 can be configured to supply electrical current to the conductive elements 114 (e.g., to stimulate) and/or receive electrical energy from the conductive elements 114 (e.g., to sense physiological data). The extension portion 106 of the lead 102 can mechanically and/or electrically couple the electronics package 108 to the lead body 104. The extension portion 106 can comprise a polymeric material such as, but not limited to, a thermoplastic elastomer, a thermoplastic polyurethane, a silicone, or other suitable materials. The extension portion 106 can be sufficiently flexible such that it can bend so as to position the lead body 104 on top of, but spaced apart from, the electronics package 108. As discussed in greater detail below with reference to FIGS. 3A-3F, the neuromodulation device 100 is configured to be implanted within both a submental region and a sublingual region such that the electronics package 108 and lead body 104 are vertically stacked with one or more muscle and/or other tissue layers positioned therebetween. The flexibility of the extension portion 106 enables such a configuration.

In some embodiments, the extension portion 106 comprises a sidewall defining a lumen extending through the extension portion 106. The conductive elements 114 can be electrically coupled to the first antenna 116 and/or the electronics component 118 via one or more electrical connections extending through the lumen of the extension portion 106. For example, the proximal end portions of the electrical connections can be routed through the first connector 110 to the electronics component 118 on the electronics package 108. The electrical connections may comprise, for example, one or more wires, cables, traces, vias, and others extending through the extension portion 106 and lead body 104. The electrical connections can comprise a conductive material such as silver, copper, etc., and each electrical connection can be insulated along all or a portion of its length. In some embodiments, the device 100 includes a separate electrical connection for each conductive element 114. For example, in those embodiments in which the device 100 comprises eight conductive elements 114 (and other embodiments), the device 100 can comprise eight electrical connections, each extending through the lumen of the extension portion 106 from a proximal end at the electronics component 118 to a distal end at one of the conductive elements 114.

In some embodiments, the electronics component 118 comprise an application-specific integrated circuit (ASIC), a discrete electronic component, and/or an electrical connector. In these and other embodiments, the electronics component 118 can comprise, for example, processing and memory components (e.g., microcomputers, microprocessors, computers-on-a-chip, etc.), charge storage and/or delivery components (e.g., batteries, capacitors, electrical conductors) for receiving, accumulating, and/or delivering electrical energy, switching components (e.g., solid state, pulse-width modulation, etc.) for selection and/or control of the conductive elements 114. In some embodiments, the electronics component 118 comprise a data communications unit for communicating with an external device (such as external system 15) via a communication standard such as, but not limited to, near-field communication (NFC), infrared wireless, Bluetooth, ZigBee, Wi-Fi, inductive coupling, capacitive coupling, or any other suitable wireless communication standard. In some examples, the electronics component 118 include one or more processors having one or more computing components configured to control energy delivery via the conductive elements 114 and/or process energy and/or data received by the conductive elements 114 according to instructions stored in the memory. The memory may be a tangible, non-transitory computer-readable medium configured to store instructions executable by the one or more processors. For instance, the memory may be data storage that can be loaded with one or more of the software components executable by the one or more processors to achieve certain functions. In some examples, the functions may involve causing the conductive elements 114 to obtain data characterizing activity of a patient's muscles. In another example, the functions may involve processing data to determine one or more parameters of the data (e.g., a change in muscle activity, etc.). According to various embodiments, the electronics component 118 can comprise a wireless charging unit for providing power to other electronics component 118 of the device 100 and/or recharging a battery of the device 100 (if included).

The electronics package 108 can also be configured to wirelessly receive energy from a power source to power the neuromodulation device 100. In some embodiments, the electronics package 108 comprises a first antenna 116 configured to wirelessly communicate with the external system 15. As shown in FIG. 2B, in some embodiments the electronics component 118 can be disposed in an opening at a central portion of the first antenna 116. In other embodiments, the electronics component 118 and antenna 116 may have other configurations and arrangements.

The second antenna 12 can be configured to emit an electromagnetic field to induce an electrical current in the first antenna 116, which can then be supplied to the electronics component 118 and/or conductive elements 114. In some embodiments, the first antenna 116 comprises a coil or multiple coils. For example, the first antenna 116 can comprise one or more coils disposed on a flexible substrate. The substrate can comprise a single substrate or multiple substrates secured to one another via adhesive materials. For instance, in some embodiments the substrate comprises multiple layers of a heat resistant polymer (such as polyimide) with adhesive material between adjacent layers. Whether comprising a single layer or multiple layers, the substrate can have one or more vias extending partially or completely through a thickness of the substrate, and one or more electrical connectors can extend through the vias to electrically couple certain electronic components of the electronics package 108, such as the first antenna 116 and/or the previously discussed electronics component 118.

In some embodiments, the first antenna 116 comprises multiple coils. For example, the first antenna 116 can comprise a first coil at a first side of the substrate and a second coil at a second side of the substrate. This configuration can be susceptible to power losses due to substrate losses and parasitic capacitance between the multiple coils and between the individual coil turns. Substrate losses occur due to eddy currents in the substrate due to the non-zero resistance of the substrate material. Parasitic capacitance occurs when these adjacent components are at different voltages, creating an electric field that results in a stored charge. All circuit elements possess this internal capacitance, which can cause their behavior to depart from that of “ideal” circuit elements.

Advantageously, in some embodiments the first antenna 116 comprises a two-layer, pancake style coil configuration in which the top and bottom coils are configured in parallel. As a result, the coils can generate an equal or substantially equal induced voltage potential when subjected to an electromagnetic field. This can help to equalize the voltage of the coils during use, and has been shown to significantly reduce the parasitic capacitance of the first antenna 116. In this parallel coil configuration, the top and bottom coils are shorted together within each turn. This design has been found to retain the benefit of lower series resistance in a two-coil design while, at the same time, greatly reducing the parasitic capacitance and producing a high maximum power output. Additional details regarding the two-coil configuration can be found in U.S. application Ser. No. 16/866,523, filed May 4, 2020, which is incorporated by reference herein in its entirety.

The first antenna 116 (or one or more portions thereof) can be flexible such that the first antenna 116 is able to conform at least partially to the patient's anatomy once implanted. In some embodiments, the first antenna 116 comprises an outer coating configured to encase and/or support the first antenna 116. The coating can comprise a biocompatible material such as, but not limited to, epoxy, urethane, silicone, or other biocompatible polymers. In some embodiments, the coating comprises multiple layers of distinct materials.

With continued reference to FIGS. 2B-2D, the lead body 104 can comprise a substrate carrying one or more conductive elements 114 configured to deliver and/or receive electrical energy. In some embodiments, the lead body 104 (or one or more portions thereof) comprises flexible tubing with a sidewall defining a lumen. The lead body 104 can comprise a polymeric material such as, but not limited to, a thermoplastic elastomer, a thermoplastic polyurethane, a silicone, or other suitable materials. The lead body 104 can comprise the same material as the extension portion 106 or a different material. The lead body 104 can comprise the same material as the extension portion 106 but with a different durometer. In some embodiments, the lead body 104 has a lower durometer than the extension portion 106, which can enhance patient comfort.

As shown in FIGS. 2B-2D, the lead body 104 has a branched shape comprising a first arm 122 and a second arm 124. To facilitate this configuration, for example, the second connector 112 can be bifurcated and/or branching. The first arm 122 and the second arm 124 can each extend distally and laterally from the second connector 112 and/or the distal end portion 106b of the extension portion 106. The first arm 122 can comprise a proximal portion 122a, a distal portion 122b, and an intermediate portion 122c extending between the proximal portion 112a and the distal portion 122b. Similarly, the second arm 124 can comprise a proximal portion 124a, a distal portion 124b, and an intermediate portion 124c extending between the proximal portion 124a and the distal portion 124b. In some embodiments, the first arm 122 can comprise a cantilevered, free distal end 123 and/or the second arm 124 can comprise a cantilevered, free distal end 125. The first arm 122 and/or the second arm 124 can include one or more fixation elements 130, for example the fixation elements 130 shown at the distal end portions 122b, 124b of the first and second arms 122, 124 in FIGS. 2B-2D. The fixation elements 130 can be configured to securely, and optionally releasably, engage patient tissue to prevent or limit movement of the lead body 104 relative to the tissue.

While being flexible, the lead 102 and/or one or more portions thereof (e.g., the lead body 104, the extension portion 106, etc.) can also be configured to maintain a desired shape. This feature can, for example, be facilitated by electrical conductors that electrically connect the conductive elements 114 carried by the lead body 104 to the electronics package 108, by an additional internal shape-maintaining (e.g., a metal, a shape memory alloy, etc.) support structure (not shown), by shape setting the substrate comprising the lead 102, etc. In any case, one or more portions of the lead 102 can have a physical property (e.g., ductility, elasticity, etc.) that enable the lead 102 to be manipulated into a desired shape or maintain a preset shape. Additionally or alternatively, the lead 102 and/or one or more portions thereof (e.g., the lead body 104, the extension portion 106, etc.) can be sufficiently flexible to at least partially conform to a patient's anatomy once implanted and/or to enhance patient comfort.

The conductive elements 114 can be carried by the sidewall of the lead body 104. For example, the conductive elements 114 can be positioned on an outer surface of the sidewall and/or within a recessed portion of the sidewall. In some embodiments, one or more of the conductive elements 114 is positioned on an outer surface of the sidewall and extends at least partially around a circumference of the sidewall. The lumen of the lead body 104 can carry one or more electrical conductors that extend through the lumen of the lead body 104 and the lumen of the extension portion 106 from the conductive elements 114 to the electronics package 108. The sidewall can define one or more apertures through which an electrical connector can extend.

Each of the conductive elements 114 may comprise an electrode, an exposed portion of a conductive material, a printed conductive material, and other suitable forms. In some embodiments, one or more of the conductive elements 114 comprises a ring electrode. The conductive elements 114 can be crimped, welded, adhered to, or positioned over an outer surface and/or recessed portion of the lead body 104. Additionally or alternatively, each of the conductive elements 114 can be welded, soldered, crimped, or otherwise electrically coupled to a corresponding electrical connector. In some embodiments, one or more of the conductive elements 114 comprises a flexible conductive material disposed on the lead body 104 via printing, thin film deposition, or other suitable techniques. Each one of the conductive elements 114 can comprise any suitable conductive material including, but not limited to, platinum, iridium, silver, gold, nickel, titanium, copper, combinations thereof, and/or others. For example, one or more of the conductive elements 114 can be a ring electrode comprising a platinum iridium alloy. In some embodiments, one or more of the conductive elements 114 comprises a coating configured to improve biocompatibility, conductivity, corrosion resistance, surface roughness, durability, or other parameter(s) of the conductive element 114. As but one example, one or more of the conductive elements 114 can comprise a coating of titanium and nitride.

In some embodiments, one or more conductive elements 114 has a length of about 1 mm. Additionally or alternatively, one or more conductive elements 114 can have a length of about 0.25 mm, about 0.5 mm, about 0.75 mm, about 1.25 mm, about 1.5 mm, about 1.75 mm, about 2 mm, about 2.25 mm, about 2.5 mm, about 2.75 mm, about 3 mm, about 3.25 mm, about 3.5 mm, about 3.75 mm, about 4 mm, about 4.25 mm, about 4.5 mm, about 4.75 mm, about 5 mm, about 6 mm, about 7 mm, about 8 mm, about 9 mm, about 10 mm, more than 10 mm, or less than 0.25 mm. In any case, adjacent conductive elements 114 carried by one of the first or second arms 122, 124 can be spaced apart along a length of the arm by about 0.25 mm, about 0.5 mm, about 0.75 mm, about 1 mm, about 1.25 mm, about 1.5 mm, about 1.75 mm, about 2 mm, about 2.25 mm, about 2.5 mm, about 2.75 mm, about 3 mm, about 3.25 mm, about 3.5 mm, about 3.75 mm, about 4 mm, about 4.25 mm, about 4.5 mm, about 4.75 mm, about 5 mm, about 6 mm, about 7 mm, about 8 mm, about 9 mm, about 10 mm, more than 10 mm, or less than 0.25 mm. The conductive elements 114 can have the same length or different lengths.

While the device 100 shown in FIGS. 2B-2D includes eight conductive elements 114 (four conductive elements 114 carried by the first arm 122 and four conductive elements 114 carried by the second arm 124), other numbers and configurations of conductive elements 114 are within the scope of the present technology. For example, the first arm 122 can carry the same number of conductive elements 114 as the second arm 124, or the first arm 122 can carry a different number of conductive elements 114 as the second arm 124. The first arm 122 and/or the second arm 124 can carry one conductive element 114, two conductive elements 114, three conductive elements 114, four conductive elements 114, five conductive elements 114, six conductive elements 114, seven conductive elements 114, eight conductive elements 114, nine conductive elements 114, ten conductive elements 114, or more than ten conductive elements 114. In some embodiments, one of the first arm 122 or the second arm 124 does not carry any conductive elements 114.

The conductive elements 114 can be configured for stimulation and/or sensing. Stimulating conductive elements 114 can be configured to deliver energy to an anatomical structure, such as, for example, a nerve or muscle. In some embodiments, the conductive elements 114 are configured to deliver energy to a hypoglossal nerve of a patient to increase the activity of the patient's tongue protrusor muscles. Sensing conductive elements 114 can be used obtain data characterizing a physiological activity of a patient (e.g., muscle activity, temperature, etc.). In some embodiments, the sensing conductive elements 114 are configured to detect electrical energy produced by a muscle of a patient to obtain EMG data characterizing an activity of the muscle. In some embodiments, the sensing conductive elements are configured to measure impedance across the conductive elements. As but one example, in some embodiments the conductive elements 114 are configured to deliver energy to a hypoglossal nerve of a patient to increase activity of the genioglossus and/or geniohyoid muscles, and obtain EMG data characterizing activity of the genioglossus muscle and/or the geniohyoid muscle of the patient. Still, the conductive elements 114 can be configured to deliver energy to and/or measure physiological electrical signals from other patient tissues.

The function that each of the conductive elements 114 is configured to perform (e.g., delivering energy to patient tissue, receiving energy from patient tissue, etc.) can be controlled by a processor of the electronics component 118 of the electronics package 108. In some embodiments, one or more of the conductive elements 114 is configured for only one of delivering energy to patient tissue or receiving energy from patient tissue. In various embodiments, one or more of the conductive elements 114 is configured for both delivering energy to patient tissue and receiving energy from patient tissue. In some embodiments, the functionality of a conductive element 114 can be based, at least in part, on an intended positioning of the device 100 within a patient and/or the position of the conductive element 114 on the lead body 104. One, some, or all of the conductive elements 114 can be positioned relative to patient tissue, such as nerves and/or muscles, so that it may be desirable for the conductive element(s) 114 to be able to both deliver energy to the patient tissue and receive energy from the patient tissue. Additionally or alternatively, some conductive elements 114 can have an intended position relative to specific patient tissues so that only delivery of stimulation energy is desired while other conductive elements 114 can have an intended position relative to specific patient tissues so that only receipt of sensing energy is desired. Advantageously, the configurations of the conductive elements 114 can be configured in software settings (which can be facilitated by electronics component 118 of the electronics package 108) so that the configurations of the conductive elements 114 are easily modifiable.

Whether configured for stimulating and/or sensing, each of the conductive elements 114 can be configured and used independently of the other conductive elements 114. Because of this, all or some of conductive elements 114, whichever is determined to be most effective for a particular implementation, can be utilized during the application of stimulation therapy. For example, one conductive element 114 of the first arm 122 can be used as a cathode while one conductive element 114 of the second arm 124 is used as an anode (or vice versa), two or more conductive elements 114 of the first arm 122 can be used (one as the cathode and one as the anode) without use of any conductive elements 114 of the second arm 124 (or vice versa), multiple pairs of conductive elements 114 of the first and second arms 122, 124 can be used, or any other suitable combination. The conductive element(s) 114 used for sensing and/or stimulation can be selected based on desired data to be collected and/or desired modulation of neural or muscle activity. For example, specific pairs of the conductive elements 114 can be used for creating an electric field tailored to stimulation of certain regions of the muscle and/or HGN that causes favorable changes in tongue position and/or pharyngeal dilation. Additionally or alternatively, conductive element(s) 114 that are positioned in contact with muscle tissue when the device 100 is implanted may be more favorable to use for EMG sensing than conductive element(s) 114 that are not positioned in contact with muscle tissue.

The lead body 104 can have a shape configured to facilitate delivery of electrical energy to a specific treatment location within a patient and/or detection of electrical energy from a sensing location within the patient. The conductive elements 114 carried by the first arm 122 can be configured to deliver electrical stimulation energy to one hypoglossal nerve (e.g., the right or the left hypoglossal nerve) of a patient and the conductive elements 114 carried by the second arm 124 can be configured to deliver electrical stimulation energy to the other hypoglossal nerve (e.g., the other of the right or the left hypoglossal nerve) of the patient.

Without being bound by theory, it is believed that increased activity of the tongue protrusor muscles during sleep reduces upper airway resistance and improves respiration. Thus, devices of the present technology are configured to deliver stimulation energy to motor nerves that control the tongue protrusors. In some embodiments, the device 100 is configured to deliver stimulation energy to the hypoglossal nerve to cause protrusion of the tongue. Additionally or alternatively, the device 100 can be configured to receive sensing energy produced by activity of one or more muscles of a patient (such as the genioglossus muscle), which can be used for closed-loop delivery of stimulation energy, evaluation of patient respiration, etc.

The device can be configured to be implanted at an anatomical region of a patient that is bound anteriorly and laterally by the patient's mandible, superiorly by the superior surface of the tongue, and inferiorly by the patient's platysma. Such an anatomical region can include, for example, a submental region and a sublingual region. The sublingual region can be bound superiorly by the oral floor mucosa and inferiorly by the mylohyoid and includes the plane between the genioglossus muscle and the geniohyoid muscle. The submental region can be bound superiorly by the mylohyoid and inferiorly by the platysma muscle. FIGS. 3A-3F depict various views of the device 100 implanted within a patient. As shown in FIGS. 3A-3F, the neuromodulation device 100 is configured to be positioned such that the electronics package 108 is disposed on or near the inferior surface of the mylohyoid in a submental region while the lead body 104 is positioned between the geniohyoid and genioglossus in a sublingual region with the arms 122, 124 disposed along the left and right hypoglossal nerves. The arms 122, 124 can be positioned such that the conductive elements 114 are disposed near the distal arborization of the hypoglossal nerves that innervate the genioglossus. In particular, the conductive elements 114 can be positioned proximate the portions of the distal arborization that innervate the horizontal fibers of the genioglossus while limiting and/or avoiding stimulation of the portions of the distal arborization of the hypoglossal nerve that activate retrusor muscles. When implanted, the extension portion 106 of the lead 102 can extend in an anterior direction away from the electronics package 108 (towards the mandible), then bend superiorly and extend through the geniohyoid muscle until bending back posteriorly and extending within a tissue plane between the geniohyoid and genioglossus muscles. In some embodiments, the extension portion 106 straddles the right and left geniohyoid muscles.

The electronics package 108 can be sufficiently flexible so that, once implanted, the electronics package 108 at least partially conforms to the curvature of the mylohyoid. Additionally or alternatively, the electronics package 108 can have a shape reflecting the curvature of the mylohyoid. In some embodiments, the electronics package 108 can comprise fixation elements (similar to fixation elements 130 or otherwise) that are configured to engage the mylohyoid (or other surrounding tissue) and prevent or limit motion of the electronics package 108 once implanted.

The lead body 104 can be configured to be positioned between the genioglossus and geniohyoid muscles of a patient so that the conductive elements 114 are positioned proximate the hypoglossal nerve. Although not shown in FIGS. 3A-3F, the hypoglossal nerve is located between the genioglossus and fascia and/or fat located between the genioglossus and the geniohyoid. In some embodiments, the lead body 104 is configured to be positioned at or just inferior to the fat between the hypoglossal nerve and the geniohyoid and thus is not positioned in direct contact with the hypoglossal nerve. In any case, once the device 100 is implanted, the lead body 104 can extend posteriorly away from the distal end portion 106b of the extension portion 106. The lead body 104 can then branch laterally such that the first arm 122 of the lead body 104 is positioned proximate one of the patient's hypoglossal nerves and the second arm 124 is positioned proximate the contralateral hypoglossal nerve. The fixation elements 130 can engage patient tissue (e.g., the fat underlying the hypoglossal nerves, etc.) to prevent or limit motion of the first and second arms 122, 124 relative to the patient tissue.

As best shown in FIG. 3C, the arms 122, 124 of the lead body 104 can bend out of the plane of the extension portion 106, in addition to extending laterally away from the extension portion 106, such that the arms 122, 124 outline a somewhat concave shape. Advantageously, this concave shape can accommodate the convex inferior surface of the genioglossus and still keep the arms 122, 124 positioned near the distal arborization of the hypoglossal nerve.

In some embodiments, conductive elements 114 are selected for use that selectively activate the protrusor muscles of a patient. In these and other embodiments, the specific positioning of the first and second arms 122, 124 relative to specific branches of the hypoglossal nerves need not be identified prior to stimulation of desired portions of the nerve and/or muscle. For example, in embodiments in which the lead body 104 includes more than two conductive elements 114, the combination of conductive elements 114 that is used for treating a patient can be selected based on physiological responses to test stimulations. For example, stimulation energy can be delivered to the hypoglossal nerve(s) via multiple combinations of conductive elements 114 and a physiological response (e.g., EMG data, tongue position, pharyngeal opening size, etc.) and/or a functional outcome (e.g., Fatigue Severity Scale, Epworth Sleepiness Scale, etc.) can be evaluated for each combination. Based on the evaluation(s), the conductive elements 114 that are selected to deliver stimulation energy can be conductive elements 114 that are associated with favorable responses/outcomes.

### IV. Selected Examples of External Devices

As previously described with reference to FIG. 2, the neuromodulation systems 10 of the present technology can include an external system 15 configured to wirelessly couple to the implantable neuromodulation device 100 (also referred to herein as “implantable device 100” or “neuromodulation device 100”), for example to provide power to the implantable device 100 and/or communicate with the implantable device 100. The external system 15 can comprise an external device 11 including a carrier 9 carrying a second antenna 12, which is communicatively coupled to a control unit 30 of the external system 15. The control unit 30 delivers RF current to the second antenna 12, and RF current flows through the second antenna 12 such that the second antenna 12 generates an electromagnetic field. When the first antenna 116 of the implantable device 100 is positioned within the electromagnetic field, an electromotive force is induced in the first antenna 116, thereby inducing an RF current in the first antenna 116, which can be used for operation of the implantable device 100.

As described herein, the implantable neuromodulation device 100 of the present technology can be configured to deliver stimulation energy to a treatment site within a patient while the patient is sleeping to stimulate the HGN and/or the genioglossus muscle to improve the patient's respiration during sleep. Accordingly, the external system 15 can be configured to provide power to the implantable device 100 while a patient is sleeping to operate the implantable device 100. In these embodiments, and others, the external device 11 can be configured to be positioned between the patient's body and a sleeping surface upon which the patient lies, reclines, and/or is otherwise positioned against during sleep. For example, the external device 11 can be configured to be positioned between the patient's head, neck, upper back, and/or another anatomical region and a surface of the patient's mattress or other suitable sleeping surface. Additionally or alternatively, the external system 15 can be configured to provide power to the implantable device 100 during and/or after implantation of the implantable device 100 (e.g., to assess positioning of the device, etc.), titration of the implantable device 100, testing of the implantable device 100, and/or in other clinical settings. The patient may or may not be asleep in such scenarios. The external device 11 can be configured to be positioned between a patient's body and a surface upon which the patient lies or reclines, such as a surface posterior to the patient when the patient is lying supine, while the implantable device 100 is being powered. In some embodiments, the external device 11 can be configured to be positioned between a vertical surface proximate the patient while the patient sits or stands upright.

The second antenna 12 carried by the carrier 9 of the external device 11 can be configured to generate an electromagnetic field having a specific magnitude and distribution such that, when the patient is positioned proximate the external device 11, the electromagnetic field provides operational power to the implantable device 100 located at a treatment site comprising submental and sublingual regions of a patient's head. According to various embodiments, operational power comprises between about 5 mW to about 50 mW, but may vary based on power requirements of the implantable device 100, a size of the first antenna 116, a design of the first antenna 116, etc.

Power is delivered to the implantable device 100 from the external system 15 by positioning the first antenna 116 within the electromagnetic field generated by the second antenna 12, which induces an electromotive force in the first antenna 116. Specifically, the change in magnetic flux (e.g., the amount of magnetic field perpendicularly penetrating the first antenna 116) induces an electromotive force in the first antenna 116. Accordingly, it is the component of the electromagnetic field perpendicular to a radial dimension of the first antenna 116 that induces an electromotive force in the first antenna 116. Because the first antenna 116 of the implantable device 100 is configured to be positioned within a submental region just inferior of a patient's mylohyoid, a radial dimension of first antenna 116 can be generally aligned with an anatomical transverse plane of the patient. Therefore, the second antenna 12 can be configured to generate an electromagnetic field having a component substantially perpendicular to the transverse plane of the patient for delivering power to the implantable device 100.

The amount of electromotive force induced in the first antenna 116 by an electromagnetic field is based at least in part on the magnitude of the component of the electromagnetic field perpendicular to the radial dimension of the first antenna 116. Thus, the second antenna 12 can be configured to generate an electromagnetic field with such a directional component having an magnitude that is sufficiently high at the location of the first antenna 116 to provide operational power to the implantable device 100. Moreover, because a patient (and thereby the first antenna 116) may move during sleep or other times of use of the implantable device 100, the second antenna 12 can be configured to generate an electromagnetic field with a desired directional component having a desired magnitude over a large, three-dimensional volume. Thus, sufficient power can be delivered to the implantable device 100 even if the patient moves or is misaligned with external device 11. Additionally, the second antenna 12 can be configured to generate an electromagnetic field having an magnitude and distribution sufficient to provide an intended amount of power to the implantable device 100 while still limiting absorption of electromagnetic radiation into patient tissues in compliance with regulatory guidelines.

FIGS. 4A, 10, 12, 15 and 18-22 illustrate representative examples of external devices 400, 1000, 1200, 1500 and 1800-2200 with various configurations in accordance with embodiments of the present technology. The features of the external devices 400, 1000, 1200, 1500 and 1800-2200 can be generally similar to the features of the external device 11 of FIG. 2, the features of the carriers 402, 1002, 1202, 1502 and 1802-2202 can be generally similar to the features of the carrier 9 of FIG. 2, the features of the antennas 404, 1004, 1204, 1504 and 1804-2204 can be generally similar to the features of the second antenna 12 of FIG. 2, etc. Like numbers (e.g., antenna 404 versus antenna 1004) are used to identify similar or identical components in FIGS. 4A, 10, 12, 15 and 18-22. The discussion of the external devices 400, 1000, 1200, 1500 and 1800-2200 of FIGS. 4A, 10, 12, 15 and 18-22 will be limited to those features that differ from one another and from the external device 11 of FIG. 2. Additionally, any of the features of the external devices 400, 1000, 1200, 1500 and 1800-2200 of FIGS. 4A, 10, 12, 15 and 18-22 can be combined with each other and/or with the features of the external device 11 of FIG. 2 and any of the features of the external device 11 of FIG. 2 can be combined with any of the features of the external devices 400, 1000, 1200, 1500 and 1800-2200 of FIGS. 4A, 10, 12, 15 and 18-22.

FIG. 4A is a planar view of an external device 400 in accordance with several embodiments of the present technology. The external device 400 can comprise a carrier 402 carrying an antenna 404. The antenna 404 can comprise conductive material such as copper, gold, silver, or another suitable metal disposed in a specific shape such that the flow of current through the conductive material causes the antenna 404 to produce an electromagnetic field. The conductive material can be etched into the carrier 402, deposited onto the carrier 402, coextruded with the carrier 402, adhered to the carrier 402, mechanically secured to the carrier 402, etc. In some embodiments, the antenna 404 includes a substrate carrying the conductive material. Such a substrate can be carried by the carrier 402. In some embodiments, the conductive material comprises a wire, a trace, a conductive tape, a conductive fabric, etc.

The antenna 404 can comprise a single layer of conductive material or multiple layers of conductive material carried by the carrier 402. For example, the antenna 404 can comprise one layer of conductive material, two layers of conductive material, three layers of conductive material, four layers of conductive material, five layers of conductive material, or more than five layers of conductive material. In embodiments in which the antenna 404 comprises multiple layers of conductive material, each of the layers can define the same shape and can be aligned with the conductive material of the adjacent layer(s). The multiple layers of conductive material can be electrically coupled to one another in parallel. In such a configuration, each layer of conductive material can be shorted together at one or more locations along a length of the conductive material, for example with electrically conductive connectors extending between adjacent layers. In some embodiments, the electrically conductive connectors comprise vias that have been drilled, laser etched, etc. Such vias can be plated and/or filled with a conductive material. Adjacent layers of conductive material can also be formed mechanically with pins, rivets, etc. In any case, connecting adjacent layers of conductive material together in parallel can reduce a series resistance of the antenna 404 relative to a single-layer design while producing a high output with low parasitic capacitance.

The carrier 402 and/or a substrate carrying the conductive material can comprise one or more flexible materials such as, but not limited to, a fabric, a foam, a tape, a polyurethane, a thermoplastic polyester, a thermoplastic elastomer, a polyimide, a rubber, etc. The carrier 402 and/or substrate can comprise a printed circuit board (PCB) substrate. For example, the carrier 402 and/or substrate can comprise FR4, CEM1, CEM3, FR2, PET, elastomers, and/or another suitable PCB substrate. The carrier 402 and/or substrate can comprise a dielectric material with good heat resistance. In some embodiments, the carrier 402 and/or substrate comprises a single layer of material. Additionally or alternatively, the carrier 402 and/or substrate can comprise multiple layers of one material, multiple layers of different materials, etc. The antenna 404 can be positioned between two layers of the carrier 402 when the carrier 402 comprises at least two layers. Additionally or alternatively, the antenna 404 can be positioned on an outer surface of the carrier 402.

According to various embodiments, the carrier 402 can comprise a shielding material configured to obstruct electromagnetic radiation in one or more directions away from the antenna 404. Such shielding material can be conductive and/or ferromagnetic. The shielding material can be configured to be positioned between the antenna 404 and the surface upon which the patient lies during use and/or testing of the implantable device (e.g., the patient's mattress or other sleeping surface during use, an operating room and/or clinic table during implantation and/or titration, etc.) and can shield the antenna 404 and the surface from one another. For example, the shielding material can be configured to shield the antenna 404 and the springs of a patient's mattress from one another. The electromagnetic field generated by the antenna 404 can induce eddy currents in the metal springs of a spring mattress. The accumulation of eddy currents in the mattress springs can cause heating of the mattress and/or can cause changes in the impedance of the antenna 404 over time and/or in a highly variable manner. The shielding material can prevent or limit the electromagnetic field generated by the antenna 404 from reaching the mattress springs, which can prevent or limit the accumulation of eddy currents in the mattress springs. Therefore, the shielding material can prevent or limit changes in the impedance of the antenna 404 caused by the surface and/or the surrounding environment. The shielding material may cause a known shift in the impedance of the antenna 404, which can be accounted for when determining the impedance of one or more components of the control unit and/or the antenna of the implantable device.

As shown in FIG. 4A, the carrier 402 has a width dimension W and a length dimension L substantially perpendicular to the width dimension W. Together, the width dimension W and the length dimension L can define a two-dimensional plane within which the antenna 404 lies. According to various embodiments, the carrier 402 can comprise a first region 402a and a second region 402b opposite the first region 402a along the length dimension L. For example, as shown in FIG. 4A, the first region 402a can be disposed on one side of a first midline M1 and the second region 402b can be disposed on the other side of the first midline M1. The first midline M1 can be defined as the location at which a direction of current flowing through the antenna 404 changes. In various embodiments, the first midline M1 of the carrier 402 can substantially bisect the carrier 402 along the length dimension L. In any case, the first region 402a and the second region 402b may not overlap one another along the length dimension L and/or along the width dimension W.

In some embodiments, the external device 400 is configured to be positioned between a patient and surface underneath the patient with the length dimension L of the carrier 402 substantially aligned with the vertical (e.g., longitudinal) axis of the patient and the width dimension W of the carrier 402 substantially aligned with the horizontal (e.g., coronal) axis of the patient. In any case, the first region 402a can be configured to be positioned proximate a first anatomical region of a patient and the second region 402b can be configured to be positioned proximate a second anatomical region of the patient. For example, the first region 402a can be configured to be positioned proximate a head of a patient and the second region 402b can be configured to be positioned proximate an upper back of a patient.

In some embodiments, the carrier 402 comprises a first broad surface and a second broad surface opposite the first broad surface along a thickness dimension of the carrier 402. The thickness dimension of the carrier 402 can be substantially orthogonal to the length dimension L and the width dimension W. Each of the first broad surface and the second broad surface can have a perimeter defining the respective surface. The perimeter of the first broad surface can be substantially similar to the perimeter of the second broad surface or the perimeter of the first broad surface can differ from the perimeter of the second broad surface. In other words, the first broad surface can have the same shape as the second broad surface or a different shape than the second broad surface. In various embodiments, the first broad surface and/or the second broad surface can have a quadrilateral shape (e.g., rectangular, square, trapezoidal, etc.). Still, the first broad surface and/or the second broad surface can have another shape such as, but not limited to, circular, ellipsoidal, triangular, hexagonal, or polygonal, or irregular. The thickness dimension of the carrier 402 can be substantially constant or can vary along the length dimension L and/or the width dimension W. A thickness of the carrier 402 can be based, at least in part, on a desired flexibility of the carrier 402. For example, when the carrier 402 is configured to be positioned underneath a patient while the patient sleeps, it may be advantageous for a thickness of the carrier 402 to be smaller so that the carrier 402 is more flexible and thereby more comfortable for the patient to sleep upon.

The antenna 404 can comprise a first portion 404a and a second portion 404b opposite the first portion 404a along the length dimension L. In various embodiments, the first portion 404a of the antenna 404 is positioned at the first region 402a of the carrier 402 and/or the second portion 404b of the antenna 404 is positioned at the second region 402b of the carrier 402. Accordingly, the first portion 404a of the antenna 404 can be configured to be positioned proximate a first anatomical region of a patient and the second portion 404b of the antenna 404 can be configured to be positioned proximate a second anatomical region of the patient. The first portion 404a and the second portion 404b can be substantially coplanar (see FIG. 4A, for example). Alternatively, the first portion 404a and the second portion 404b can be positioned on distinct planes from one another along the thickness dimension of the carrier 402. Such distinct planes may or may not be substantially parallel.

A length of the antenna 404 along the length dimension L can be between about 20 cm and about 100 cm, between about 30 cm and about 90 cm, between about 40 cm and about 80 cm, between about 50 cm and about 70 cm, about 20 cm, about 30 cm, about 40 cm, about 50 cm, about 60 cm, about 70 cm, about 80 cm, about 90 cm, about 100 cm, or another suitable length. A width of the antenna 404 along the width dimension W can be between about between about 20 cm and about 100 cm, between about 30 cm and about 90 cm, between about 40 cm and about 80 cm, between about 50 cm and about 70 cm, about 20 cm, about 30 cm, about 40 cm, about 50 cm, about 60 cm, about 70 cm, about 80 cm, about 90 cm, about 100 cm.

The antenna 404 can comprise one or more lengths of conductive material forming an inductor, e.g., the flow of current through the conductive material causes the antenna 404 to produce a magnetic field. In various embodiments, the first portion 404a of the antenna 404 comprises a first length 406 of conductive material and the second portion 404b of the antenna 404 comprises a second length 408 of conductive material. The first length 406 can be greater than the second length 408 (see FIG. 4A, for example), the same as the second length 408, or less than the second length 408. The relative lengths of conductive material at the first portion 404a of the antenna 404 and the second portion 404b of the antenna 404 can influence the spatial distribution of a magnetic field generated by the antenna 404.

As shown in FIG. 4A, the first length 406 of conductive material can extend from a first end 410 to a second end 412 and/or the second length 408 of conductive material can extend from a first end 414 to a second end 416. In some embodiments, the first length 406 forms a first loop 418 and/or the second length 408 forms a second loop 420. The first end 410 of the first length 406 can be located at the same position as the second end 412 of the first length 406 and/or the first end 414 of the second length 408 can be located at the same position as the second end 416 of the second length 408. In some embodiments, the first end 410 of the first length 406, the second end 412 of the first length 406, the first end 414 of the second length 408, and the second end 416 of the second length 408 are located at the same position along the width dimension W and/or the length dimension L of the antenna 404. In these embodiments, and others, the first end 410 of the first length 406 and the first end 414 of the second length 408 can be located at a first position along the thickness dimension of the carrier 402 while the second end 412 of the first length 406 and the second end 416 of the second length 408 are located at a second position along the thickness dimension of the carrier 402 that differs from the first position. The first length 406 of conductive material and the second length 408 of conductive material can be electrically coupled to one another in series (e.g., via connection of the first ends 410, 414 and connection of the second ends 412, 416).

The first length 406 of conductive material can comprise a plurality of regions extending substantially along the width dimension W and/or the length dimension L. As shown in FIG. 4A, one or more ends of each region can be curved (e.g., to transition from a region extending along the width dimension W to a sequential region extending along the length dimension L, to transition from a region extending along the length dimension L to a sequential region extending along the width dimension W, etc.). The first length 406 can comprise a first region 406a extending from a first end of the first region 406a at the first end 410 of the first length 406 to a second end of the first region 406a in a first length direction along the length dimension L, a second region 406b extending from a first end of the second region 406b at the second end of the first region 406a to a second end of the second region 406b in a first width direction along the width dimension W, a third region 406c extending from a first end of the third region 406c at the second end of the second region 406b to a second end of the third region 406c in the first length direction, a fourth region 406d extending from a first end of the fourth region 406d at the second end of the third region 406c to a second end of the fourth region 406d in a second width direction along the width dimension W and opposite the first width direction, a fifth region 406e extending from a first end of the fifth region 406e at the second end of the fourth region 406d to a second end of the fifth region 406e in a second length direction along the length dimension L and opposite the first length direction, a sixth region 406f extending from a first end of the sixth region 406f at the second end of the fifth region 406e to a second end of the sixth region 406f in the first width direction, and a seventh region 406g extending from a first end of the seventh region 406g at the second end of the sixth region 406f to a second end of the seventh region 406g in the first length direction.

Collectively, the fourth-seventh regions 406d-406g can form a first subloop 422a. The first subloop 422a can enclose a smaller area than an area enclosed by the first loop 418. In some embodiments, for example as shown in FIG. 4A, the second region 406b spans a greater distance along the width dimension W than the fourth region 406d and/or the sixth region 406f. In various embodiments, the fifth region 406e spans a smaller distance along the length dimension L than the third region 406c and/or the seventh region 406g. The sixth region 406f can be spaced apart from the first end 410 of the first length 406 and/or the second region 406b of the first length 406 along the length dimension L, which can facilitate the generation of a magnetic field having at least a minimum desired intensity over a range of distances away from the carrier 402 along the thickness dimension corresponding to expected positions of the implantable device. This is because the distribution of the magnetic field generated from current flowing through the first subloop 422a at least partially depends on a distance between the sixth region 406f region and the first midline M1 along the length dimension L.

As shown in FIG. 4A, the first length 406 can comprise an eighth region 406h extending from a first end of the eighth region 406h at the second end of the seventh region 406g to a second end of the eighth region 406h in the second width direction. The eighth region 406h can span a greater distance along the width dimension W than the second region 406b, the fourth region 406d, and/or the sixth region 460f. For example, the eighth region 406h can extend across a second midline M2 of the carrier 402. The second midline M2 can bisect the carrier 402 along the width dimension W. Additionally or alternatively, the second midline M2 can be defined by a location of a feed 426 (schematically represented by an arrow in FIG. 4A) at which the antenna 404 electrically couples to the control unit. In some embodiments, a shape formed by the first length 406 of conductive material is symmetric about the second midline M2.

As shown in FIG. 4A, the first length 406 can comprise a ninth region 406i extending from a first end of the ninth region 406i at the second end of the eighth region 406h to a second end of the ninth region 406i in the second length direction, a tenth region 406j extending from a first end of the tenth region 406j at the second end of the ninth region 406i to a second end of the tenth region 406j in the first width direction, an eleventh region 406k extending from a first end of the eleventh region 406k at the second end of the tenth region 406j to a second end of the eleventh region 406k in the first length direction, and a twelfth region 406l extending from a first end of the twelfth region 406l at the second end of the eleventh region 406k to a second end of the twelfth region 406l in the second width direction. Collectively, the ninth-twelfth regions 406i-406l can form a second subloop 422b. The second subloop 422b can enclose an area that is smaller than an area enclosed by the first loop 418. In some embodiments, the second subloop 422b formed by the ninth-twelfth regions 406i-406l encloses the same area as the first subloop 422a formed by the fourth-seventh regions 406d-406g. In some embodiments, the first subloop 422a is spaced apart from the second subloop 422b along the width dimension W. For example, the fifth region 406e and the eleventh region 406k can be spaced apart along the width dimension W.

The first length 406 can comprise a thirteenth region 406m extending from a first end of the thirteenth region 406m at the second end of the twelfth region 406l to a second end of the thirteenth region 406m in the second length direction, a fourteenth region 406n extending from a first end of the fourteenth region 406n at the second end of the thirteenth region 406m to a second end of the fourteenth region 406n in the first width direction, and a fifteenth region 406o extending from a first end of the fifteenth region 406o at the second end of the fourteenth region 406n to a second end of the fifteenth region 406o at the second end 412 of the first length 406 in the second length direction.

According to various embodiments, the antenna 404 can be asymmetric about the first midline M1 of the carrier 402. For example, the second length 408 of conductive material can have a different total length than the first length 406 of conductive material, the second portion 404b of the antenna 404 can have a different density of conductive material than the first portion 404a, the second length 408 can form a different number of loops than the first length 406, the second length 408 can have a different shape than the first length 406, etc. Such asymmetry can facilitate the antenna 404 being configured to generate an intended magnetic field having sufficient magnitude to power an implantable device near the external device 400 without exposing the patient to excessive electromagnetic radiation, as described in greater detail below.

As shown in FIG. 4A, the second length 408 can comprise a first region 408a extending from a first end of the first region 408a at the first end 414 of the second length 408 to a second end of the first region 408a in the second length direction, a second region 408b extending from a first end of the second region 408b at the second end of the first region 408a to a second end of the second region 408b in the second width direction, a third region 408c extending from a first end of the third region 408c at the second end of the second region 408b to a second end of the third region 408c in the second length direction, a fourth region 408d extending from a first end of the fourth region 408d at the second end of the third region 408c to a second end of the fourth region 408d in the first width direction, a fifth region 408e extending from a first end of the fifth region 408e at the second end of the fourth region 408d to a second end of the fifth region 408e in the first length direction, a sixth region 408f extending from a first end of the sixth region 408f at the second end of the fifth region 408e to a second end of the sixth region 408f in the second width direction, and a seventh region 408g extending from a first end of the seventh region 408g at the second end of the sixth region 408f to a second end of the seventh region 408g at the second end 416 of the second length 408 in the first length direction. Collectively, the first-seventh regions 408a-408g of the second length 408 can form the second loop 420.

The first length 406 and the second length 408 can be shaped so that the antenna 404 is configured to generate an intended magnetic field when RF current flows through the antenna 404. For example, the first length 406 and the second length 408 can be shaped such that the antenna 404 has a large quadrupole moment and a small dipole moment such that the antenna 404 is configured to produce an electromagnetic field with little electromagnetic radiation at far distances from the antenna 404. The dipole and quadrupole moments of the antenna 404 can be at least partially based on an area enclosed by the first loop 418, the second loop 420, the first subloop 422a, and/or the second subloop 422b. For example, a total area enclosed by the first loop 418, the first subloop 422a, and the second subloop 422b can be substantially equivalent to an area enclosed by the second loop 420 so that the antenna 404 has a large quadrupole moment and a small dipole moment. Additionally or alternatively, the dipole and quadrupole moments of the antenna 404 can be at least partially based on a distance between the first midline M1 and the first loop 418, the second loop 420, the first subloop 422a, and/or the second subloop 422b.

In some embodiments, the second region 408b of the second length 408 and the sixth region 408f of the second length 408 can be spaced apart from the fourth region 408d of the second length 408 along the length dimension L. For example, the second region 408b of the second length 408 and the sixth region 408f of the second length 408 can be spaced apart from the fourth region 408d of the second length 408 along the length dimension L by about 19 cm. The third region 408c of the second length 408 can be separated from the fifth region 408e of the second length 408 along the width dimension W. For example, the third region 408c of the second length 408 can be separated from the fifth region 408e of the second length 408 along the width dimension W by about 70 cm. Accordingly, the second loop 420 can enclose an area of about 1330 cm².

The second region 406b and the fourteenth region 406n of the first length 406 of conductive material can be spaced apart from the sixth region 408f and the second region 408b, respectively, of the second length 408 of conductive material along the length dimension L. For example, the second region 406b and the fourteenth region 406n can be spaced apart from the sixth region 408f and the second region 408b, respectively, along the length dimension L by about 8.5 cm. In some embodiments, the second region 406b and the fourteenth region 406n can be spaced apart from the sixth region 408f and the second region 408b, respectively, along the length dimension L by between about 1 cm and about 15 cm, between about 5 cm and about 10 cm, etc. A distance between the second region 406b and the sixth region 408f and/or a distance between the fourteenth region 406n and the second region 408b can be increased to increase a distance from the external device 400 perpendicular to the plane of the antenna 404 within which a magnitude of a horizontal component of the magnetic field is sufficiently high to provide power to the implantable device.

In some embodiments, the second region 406b of the first length 406 can be spaced apart from the sixth region 406f of the first length 406 along the length dimension L and/or the fourteenth region 406n of the first length 406 can be spaced apart from the tenth region 406j of the first length 406 along the length dimension L. For example, the second region 406b of the first length 406 can be spaced apart from the sixth region 406f of the first length 406 along the length dimension L and/or the fourteenth region 406n of the first length 406 can be spaced apart from the tenth region 406j of the first length 406 along the length dimension L by about 7.5 cm. In some embodiments, the second region 406b of the first length 406 can be spaced apart from the sixth region 406f of the first length 406 along the length dimension L and/or the fourteenth region 406n of the first length 406 can be spaced apart from the tenth region 406j of the first length 406 along the length dimension L by between about 1 cm and about 15 cm, between about 5 cm and about 10 cm, etc. A distance between the second region 406b of the first length 406 can be spaced apart from the sixth region 406f of the first length 406 along the length dimension L and/or the fourteenth region 406n of the first length 406 can be spaced apart from the tenth region 406j of the first length 406 along the length dimension L can be increased to increase a distance from the external device 400 perpendicular to the plane of the antenna 404 within which a magnitude of a horizontal component of the magnetic field is sufficiently high to provide power to the implantable device

The fourth region 406d of the first length 406 and/or the twelfth region 406l of the first length 406 can be spaced apart from the eighth region 406h of the first length 406 along the length dimension L. For example, the fourth region 406d of the first length 406 and/or the twelfth region 406l of the first length 406 can be spaced apart from the eighth region 406h of the first length 406 along the length dimension L by about 10 cm. In some embodiments, the fourth region 406d of the first length 406 and/or the twelfth region 406l of the first length 406 can be spaced apart from the eighth region 406h of the first length 406 along the length dimension L by between about 1 cm and about 15 cm, between about 5 cm and about 10 cm, etc.

As shown in FIG. 4A, the third region 406c of the first length 406 can be spaced apart from the seventh region 406g of the first length 406 along the width dimension W and/or the ninth region 406i of the first length 406 can be spaced apart from the thirteenth region 406m of the first length 406 along the width dimension W. For example, the third region 406c of the first length 406 can be spaced apart from the seventh region 406g of the first length 406 along the width dimension W and/or the ninth region 406i of the first length 406 can be spaced apart from the thirteenth region 406m of the first length 406 along the width dimension W by about 2 cm. In some embodiments, the third region 406c of the first length 406 can be spaced apart from the seventh region 406g of the first length 406 along the width dimension W and/or the ninth region 406i of the first length 406 can be spaced apart from the thirteenth region 406m of the first length 406 along the width dimension W by between about 0.1 cm and about 5 cm, between about 0.5 cm and about 1 cm, etc.

In some embodiments, for example as shown in FIG. 4A, the first region 406a of the first length 406 can be spaced apart from the fifteenth region 406o of the first length 406 along the width dimension W and/or the first region 408a of the second length 408 can be spaced apart from the seventh region 408g of the second length 408 along the width dimension W. For example, the first region 406a of the first length 406 can be spaced apart from the fifteenth region 406o of the first length 406 along the width dimension W and/or the first region 408a of the second length 408 can be spaced apart from the seventh region 408g of the second length 408 along the width dimension W by about 2 cm. In some embodiments, the first region 406a of the first length 406 can be spaced apart from the fifteenth region 406o of the first length 406 along the width dimension W and/or the first region 408a of the second length 408 can be spaced apart from the seventh region 408g of the second length 408 along the width dimension W by between about 0.1 cm and about 5 cm, between about 0.5 cm and about 1 cm, etc.

As previously noted, the first end 410 of the first length 406 can be connected to the first end 414 of the second length 408 and the second end 412 of the first length 406 can be connected to the second end 416 of the second length 408. In some embodiments, the first ends 410, 414 are located at a different position along the thickness of the carrier 402 than the second ends 412, 416 so that the first ends 410, 414 meet at the same location along the width dimension W and the length dimension L of the carrier 402 as the second ends 412, 416 (e.g., the first ends 410, 414 overlie the second ends 412, 416, or the second ends 412, 416 overlie the first ends 410, 414). In some embodiments, the location where the first ends 410, 414 and the second ends 412, 416 meet can be located in a portion of the external device 400 referred to herein as a “transition region.” The transition region can include at least a portion of the first region 406a of the first length 406, at least a portion of the fifteenth region 406o of the first length 406, at least a portion of the first region 408a of the second length 408, and at least a portion of the seventh region 408g of the second length 408. Within the transition region, RF current can flow in a first direction (e.g., clock-wise or counter-clockwise) between the first region 406a of the first length 406 and the first region 408a of the second length 408 and RF current can flow in a second direction opposite the first direction between the fifteenth region 406o of the first length 406 and the seventh region 408g of the second length 408. Generally, the high density of conductive material in the transition region can undesirably result in an increased combined magnitude and flux of the magnetic field in the transition region, and thus in an increased amount of SAR. However, the magnetic field magnitude in the transition region can be reduced by positioning closely together those portions of the second antenna in which RF current travels in opposite directions. For example, a portion of the second antenna extending between the first region 406a of the first length 406 and the first region 408a of the second length 308 (where RF current flows in a first direction) can be located close in proximity to the portion of the second antenna extending between the fifteenth region 406o of the first length 406 and the seventh region 408g of the second length 408 (where RF current flows in a second direction opposite the first direction). The closer in proximity these portions of the second antenna with opposite-flowing RF currents are, the more their respective contributions toward magnetic field magnitude cancel each other out, thereby reducing the overall effect of the transition region to the total SAR.

Close proximity can be achieved, for example, by positioning these two portions of the second antenna (with opposite-flowing RF currents) close together as measured along the thickness direction, along the width dimension and/or along the length direction. In some embodiments, some or all of these portions of the second antenna within the transition region can overlie one another along the thickness of the carrier (e.g., within 2 cm, within 1 cm, within 0.5 cm, within 0.25 cm, etc. as measured along the thickness direction).

In some embodiments, in the transition region, at least a portion of at least one of the first region 406a of the first length 406, the fifteenth region 406o of the first length 406, the first region 408a of the second length 408, or the seventh region 408g of the second length 408 extends diagonally away from its corresponding end along the width dimension W and/or the length dimension L of the carrier 402. For example, as shown in FIG. 4B, the first region 406a of the first length 406 can have a first portion 406a1 that extends diagonally away from the first end 410 of the first length 406 along the length and width dimensions L, W and a second portion 406a2 that extends substantially straight along the length dimension L away from the first portion 406a1 and/or the fifteenth region 406o of the first length 406 can have a first portion 406o1 that extends diagonally away from the second end 412 of the first length 406 along the length and width dimensions L, W and a second portion 406o2 that extends substantially straight along the length dimension L away from the first portion 406o1. Additionally or alternatively, the first region 408a of the second length 408 can have a first portion 408a1 that extends diagonally away from the first end 414 of the second length 408 along the length and width dimensions L, W and a second portion 408a2 that extends substantially straight along the length dimension L away from the first portion 408a1 and/or the seventh region 408g of the second length 408 can have a first portion 408g1 that extends diagonally away from the second end 416 of the second length 408 along the length and width dimensions L, W and a second portion 408g2 that extends substantially straight along the length dimension L away from the first portion 408g1. As shown in FIG. 4B, an angle β can be defined between the first portion 406a1 of the first region 406a of the first length 406 and the first portion 406o1 of the fifteenth region 406o of the first length 406.

As discussed above, the magnetic field produced by the antenna 404 in the transition region can be high at least in part because of the high density of conductive material in this region, and it may be desirable to decrease the magnitude of the magnetic field in this region, for example to prevent excessive SAR in an anatomical region of a patient positioned proximate this region of the antenna 404. With reference to FIGS. 4B and 4C, according to various embodiments, the angle β in the transition region can be reduced to position more closely together those portions of the second antenna in which RF current flow in opposite directions. As the angle β increases (e.g., FIG. 4B), the magnetic field generated by the flow of RF current in the first direction between the first regions 406a, 408a is additive with the magnetic field generated by the flow of RF current in the second direction between the fifteenth and seventh regions 406o, 408g. However, as the angle β decreases (e.g., FIG. 4C) and the first regions 406a, 408a and the fifteenth and seventh regions 406o, 408g approach parallel with the length dimension L, more of the magnetic field generated by the flow of current in the first direction between the first regions 406a, 408a tends to cancel out the magnetic field generated by the flow of current in the second direction between the fifteenth and seventh regions 406o, 408g, thereby reducing the magnitude of the magnetic field produced at the transition region of the antenna 404. In some embodiments, for example, the angle β can be less than about 45 degrees, less than about 30 degrees, less than about 15 degrees, less than about 10 degrees, less than about 5 degrees, or less than about 2 degrees. In some embodiments, the angle β can be between about 2 degrees and about 45 degrees, between about 2 degrees and about 30 degrees, or between about 5 degrees and about 15 degrees. In some embodiments, the angle β can be substantially zero degrees. For example, some or all of the portions of the antenna 404 in the transition region in which RF current travels in opposite directions can be parallel (e.g., adjacent to each other in the width-length plane of the external device, or overlying each other along the thickness dimension of the external device. In combination with being positioned at any of these angles β, the portions of the antenna 404 in the transition region in which RF current travels in opposite direction can also be positioned close to each other as measured along the thickness direction of the carrier (e.g., within 2 cm, within 1 cm, within 0.5 cm, within 0.25 cm, etc. as measured along the thickness direction).

According to various embodiments, the antenna 404 can be configured to electrically couple to a control unit at a feed 426. The control unit can deliver current to the antenna 404 via the feed 426. The feed 426 can comprise two terminals such that current is delivered to the antenna 404 from the control unit at a first terminal of the feed 426 and received by the control unit from the antenna 404 at a second terminal of the feed 426. As shown in FIG. 4A, in some embodiments the feed 426 is at the eighth region 406h of the first length 406 of conductive material. Accordingly, the eighth region 406h can be discontinuous at the feed 426. The feed 426 can be located at any suitable region of the first length 406 and/or the second length 408. Although FIG. 4A depicts one feed 426, the antenna 404 can comprise more than one feed 426 (e.g., two feeds 426, three feeds 426, four feeds 426, etc.). In some examples, two or more of the first loop 418, the second loop 420, the first subloop 422a, or the second subloop 422b can receive current from a distinct feed 426. Delivery of current to the antenna 404 via multiple feeds 426 can require less power at each feed 426 and reduce the voltage across the antenna 404, but may require more complex electronics at the control unit.

The antenna 404 can comprise one or more capacitors 428 electrically coupled to the first length 406, the second length 408, and/or the feed 426. One, some, or all of the capacitors 428 can be electrically coupled to the conductive material in series and/or in parallel. The capacitors 428 can be configured to resonate the antenna 404 partially or completely, which can facilitate the transfer of power into the antenna 404 and/or to an implantable device. Additionally, the capacitors 428 reduce or limit the peak electric field along the antenna 404. In various embodiments, the capacitors 428 each have a capacitance based on an inductance of the conductive material, an impedance of the conductive material, and/or a resonant frequency of the antenna 404. For example, one or more of the capacitors 428 can have a capacitance of about 100 pF, about 200 pF, about 300 pF, about 400 pF, about 500 pF, about 600 pF, about 700 pF, about 800 pF, about 900 pF, about 1000 pF, about 1100 pF, about 1200 pF, about 1300 pF, about 1400 pF, about 1500 pF, about 1600 pF, about 1700 pF, about 1800 pF, about 1900 pF, about 2000 pF, between about 500 pF and about 2000 pF, between about 600 pF and about 1900 pF, between about 700 pF and about 1800 pF, between about 800 pF and about 1700 pF, between about 900 pF and about 1600 pF, between about 1000 pF and about 1500 pF, between about 1100 pF and about 1400 pF, or between about 1200 pF and about 1300 pF. In some embodiments, a capacitance of one or more of the capacitors 428 can be tunable.

The capacitance value of each of the capacitors 428 can be based at least in part on an intended presented impedance of the antenna 404 and/or an impedance of one or more components of a control unit configured to electrically couple with the antenna 404, which can facilitate power transfer from the power supply to the antenna 404 via the control unit. The maximum power transfer theorem provides that a maximum amount of power can be delivered from a source (e.g., the control unit) to a load (e.g., the antenna 404) when the impedance of the source matches the impedance of the load. An impedance of the antenna 404 is based on the resistance, capacitance, inductance, and reactance of the antenna 404. Thus, in some embodiments, the capacitors 428 of the antenna can have capacitance values based on an intended impedance of the antenna 404, which can correspond to an impedance of the one or more components of the control unit.

However, even if the impedance of the antenna is initially matched to the impedance of the one or more components of the control unit, the impedance of the antenna can fluctuate with changes in environment (e.g., temperature, surface upon which the external device is positioned, weight on the external device, etc.). The impedance of the antenna can change, for example, if a patient lies on the mat, if the patient moves relative to the mat, if a foreign object is positioned over the mat, etc. The capacitors 428 of the antenna 404 can help accommodate such changes in impedance of the antenna 404. Generally, a matching circuit (such as those described herein) has a greater ability to compensate for changes in the real portion of the impedance of the antenna 404 than changes in the imaginary portion of the impedance of the antenna 404. Thus, in some embodiments, the capacitors 428 can help stabilize (e.g., prevent or limit changes in) the imaginary portion of the impedance of the antenna 404 during operation (e.g., in response to patient movement, presence of foreign objects) while the matching circuit accommodates changes in the real portion of the impedance, thereby optimizing the presented impedance of the antenna 404.

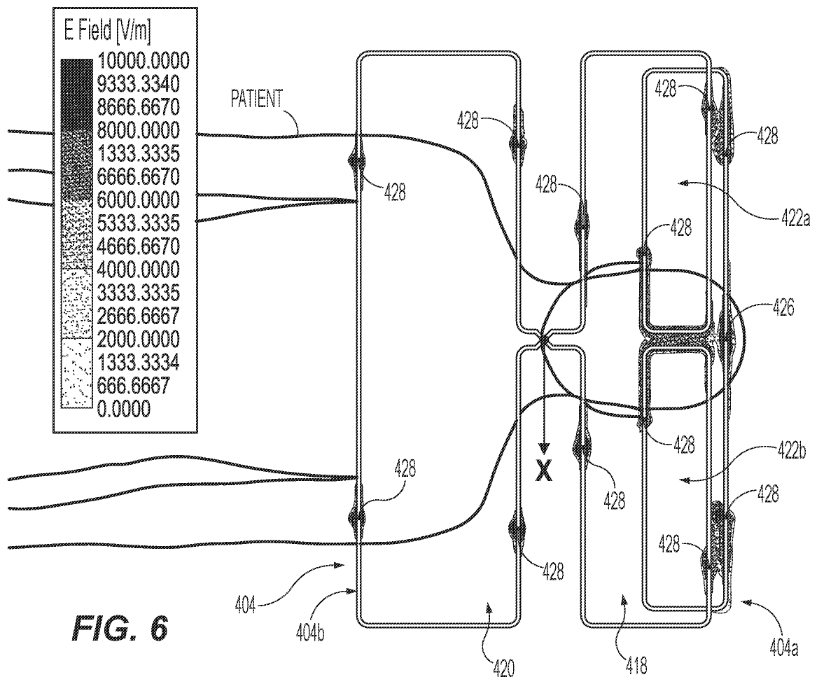

As shown in FIG. 4A, the antenna 404 can include a plurality of capacitors 428. For example, the antenna 404 can include two capacitors 428, three capacitors 428, four capacitors 428, five capacitors 428, six capacitors 428, seven capacitors 428, eight capacitors 428, nine capacitors 428, 10 capacitors 428, 11 capacitors 428, 12 capacitors 428, 13 capacitors 428, 14 capacitors 428, 15 capacitors 428, 16 capacitors 428, 17 capacitors 428, 18 capacitors 428, 19 capacitors 428, 20 capacitors 428, or more. In some embodiments, the antenna 404 comprises a single capacitor 428. Some or all of the capacitors 428 can be substantially equally spaced apart along the conductive material of the antenna 404. In some embodiments, some or all of the capacitors 428 are unequally spaced apart along the first length 406 and/or the second length 408. As discussed in greater detail below with reference to FIG. 6, the capacitors 428 can be spaced apart along the conductive material such that a peak voltage of the antenna 404 does not exceed a predetermined threshold. In various embodiments, a peak voltage of the antenna 404 does not exceed about 400 V, about 500 V, about 600 V, or about 700 V.

In some embodiments, the antenna 404 comprises one or more resistor-capacitor (RC) networks (not shown in FIG. 4A) electrically coupled to the conductive material of the antenna 404 to reduce or eliminate parasitic resonance of the antenna 404. Parasitic resonances of the antenna 404 can occur because of the coiled shape of the antenna 404, particularly at higher frequencies. For example, the location at which the first and second ends 410, 412 of the first length 406 meet the first and second ends 414, 416 of the second length 408 can be capacitive and can interact with the inductive second length 408 forming the second loop 420 to generate a parasitic resonance. Positioning an RC network between the first region 408a and the seventh region 408g of the second length 408 can reduce or eliminate the quality factor (Q factor) of this parasitic resonance. It can be useful to reduce or eliminate parasitic resonances for complying with emission regulations, for example. An RC network can be positioned at a specific location along the first length 406 and/or the second length 408 based on the geometry of the antenna 404 and/or the intended use environment of the antenna 404.

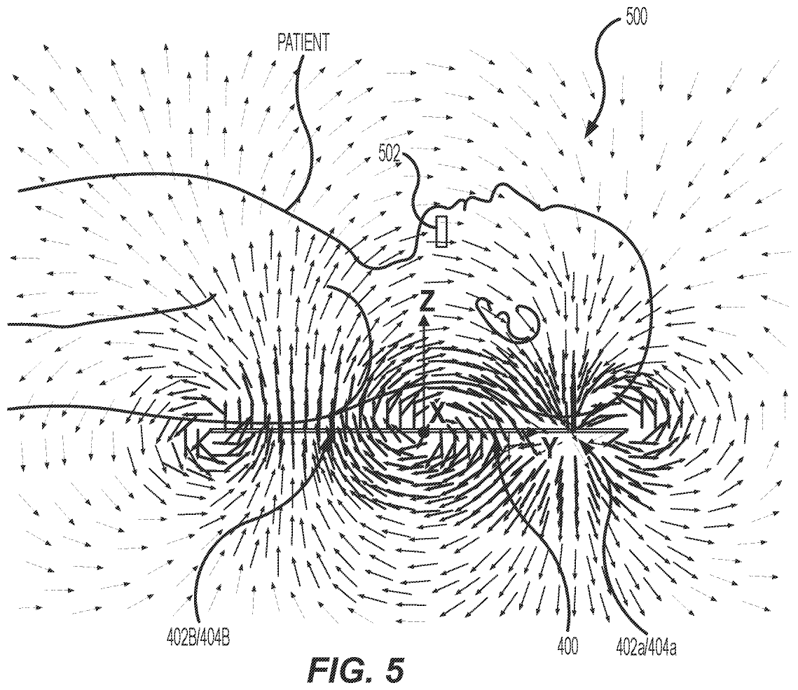

FIG. 5 illustrates a sagittal view of a simulated magnetic field 500 generated by the antenna 404 of the external device 400 of FIG. 4A. A patient is shown positioned over the external device 400 in FIG. 5 for reference. As shown in FIG. 5, in some cases the external device 400 is configured to be positioned at a posterior side of the patient. For example, the patient can be lying supine (FIG. 5) and the external device 400 can be positioned underneath the patient. In any case, an antenna 502 of an implantable device positioned at a treatment site within submental and sublingual regions of the patient's head can be oriented with a radial dimension of the antenna 502 substantially perpendicular to the width and length dimensions of the antenna 404 of the external device 400.

An RF current delivered to the antenna 404 at the feed 426 can flow through the first length 406 in a first direction and can flow through the second length 408 in a second direction opposite the first direction. A first magnetic field is generated from the RF current flowing through the first length 406 and a second, opposite magnetic field is generated from current flowing through the second length 408. The first magnetic field and the second magnetic field can interfere, e.g., constructively and/or destructively, to form the magnetic field 500 generated by the antenna 404. The magnetic field 500 can have a horizontal component that is substantially parallel to a plane containing the first length 406 and the second length 408 of the antenna 404 (e.g., defined by the width and length dimensions of the antenna 404, substantially parallel to a broad surface of the carrier 402, etc.). When the external device 400 is positioned posterior of the patient with the implantable device antenna 502 angled with respect to the plane containing the first length 406 and the second length 408, the horizontal component of the magnetic field 500 can be substantially perpendicular to the implantable device antenna 502 and can induce an electromotive force in the implantable device antenna 502. In some embodiments, the magnetic field is substantially horizontal in orientation at a location that is substantially aligned with the first and second ends 410, 412, 414, 416 of the first and second lengths 406, 408 (e.g., a transition region between the first loop 418 and the second loop 420) along the length dimension L and the width dimension W of the carrier 402.

FIG. 5 illustrates the magnitude, density, and direction of the magnetic field 500, represented by arrows, at various points within a sagittal plane. The width and dash pattern of the lines of the arrows generally indicate the magnitude of the magnetic field 500 at the locations of the arrows (e.g., the thickest, solid arrows represent the locations at which the magnetic field 500 has the highest magnitude, while the thinnest, dashed arrows represent the locations at which the magnetic field 500 has the lowest magnitude, etc.). As shown in FIG. 5, a density of the magnetic field 500 (and thus amount of magnetic flux) can be greatest near the antenna 404 and can decrease farther away from the antenna 404. Specifically, the density of the magnetic field 500 can decrease with increasing distance away from the antenna 404 along a height dimension orthogonal to the length dimension L and width dimension W of the antenna 404 and/or the density of the magnetic field 500 can decrease with increasing distance from the antenna 404 along the width dimension W of the antenna 404. In some embodiments, the density of the magnetic field 500 can be larger at the first region 402a of the carrier 402 and/or the first portion 404a of the antenna 404 than at the second region 402b of the carrier 402 and/or the second portion 404b of the antenna 404. As discussed in greater detail below, such distribution of the magnetic field 500 can facilitate delivery of an intended amount of power to the implantable device antenna 502 while preventing or limiting absorption of electromagnetic radiation into the patient's tissues. The preferential distribution of the magnetic field 500 towards the first portion 404a of the antenna 404 can be based at least in part on an asymmetry of the antenna 404 about the first midline M1.

FIG. 6 is a coronal view of an intensity map of an electric field produced by the antenna 404. Specifically, FIG. 6 depicts the electric field in the plane in which the antenna 404 lies. As current flows through the conductive material of the antenna 404, a voltage in the antenna 404 increases proportionally with distance from the feed 426. As the electric field is a function of the voltage in the antenna 404, the electric field also increases with distance from the feed 426. However, the voltage across each capacitor 428 is substantially equivalent in magnitude and opposite in polarity to the voltage along a length of the conductive material preceding the capacitor 428. Thus, the capacitor 428 voltage counteracts the voltage across a length of conductive material, and the voltage across a discrete length of material between capacitors 428 is substantially less than a voltage across the entire length of conductive material of the antenna 404. Limiting the voltage across the antenna 404 has significant benefits for ensuring patient safety, enhancing the robustness of the system to variations in load on the antenna 404, and reducing the manufacturing cost of the antenna 404.

As previously noted, the antenna 404 of the external device 400 is configured to generate an electromagnetic field having a specific magnitude and distribution so that power is delivered to an implantable device located at a treatment site comprising a submental region and a sublingual region of a patient when the patient is positioned proximate the external device 400. However, such an electromagnetic field must also have a specific magnitude and distribution such that absorption of energy from the electromagnetic field into patient tissues is limited according to regulatory guidelines. For example, the U.S. Federal Communication Commission requires that in a general population with uncontrolled electromagnetic exposure, the specific absorption rate (SAR) at which energy is absorbed per unit mass by a patient's entire body must not exceed 0.08 W/kg and that the peak special-average SAR (psSAR) at which energy is absorbed over any 1 g of tissue must not exceed 1.6 W/kg (47 CFR § 1.1310). Thus, the antenna 404 can be configured to produce a magnetic field that does not cause energy absorption into patient tissues above one or more regulatory thresholds.

When a magnetic field produced by the antenna 404 is powering an implant, a SAR parameter within patient tissue may not exceed a predetermined threshold. The SAR parameter can comprise a psSAR averaged over any 1 gram of tissue of the patient (except at the extremities of the patient), a psSAR averaged over any 10 grams of tissue at extremities of the patient, and/or an average SAR averaged over an entire body of the patient. The predetermined threshold can be based at least in part on whether the exposure to the magnetic field created by the antenna 404 is controlled or uncontrolled exposure. For example, for uncontrolled exposure, the SAR parameter can comprise a psSAR averaged over any 1 gram of tissue of the patient, except at extremities of the patient, and averaged over no more than 30 minutes, and the predetermined threshold can comprise 1.6 W/kg. Yet for controlled exposure, the SAR parameter can comprise a psSAR averaged over any 1 gram of tissue, except at extremities of the patient, and averaged over no more than 6 minutes, and the predetermined threshold can comprise 8 W/kg. For uncontrolled exposure, the SAR parameter can comprise a psSAR averaged over any 10 grams of tissue at extremities of the patient, and averaged over no more than 30 minutes, and the predetermined threshold can comprise 4 W/kg whereas for controlled exposure, the SAR parameter can comprise a psSAR averaged over any 10 grams of tissue at extremities of the patient, and averaged over no more than 6 minutes, and the predetermined threshold can comprise 20 W/kg. In some embodiments, for uncontrolled exposure, the SAR parameter can comprise an average SAR averaged over an entire body of the patient and averaged over no more than 30 minutes, and the predetermined threshold can comprise 0.08 W/kg whereas for controlled exposure, the SAR parameter can comprise an average SAR averaged over an entire body of the patient and averaged over no more than 6 minutes, and the predetermined threshold can comprise 0.4 W/kg.

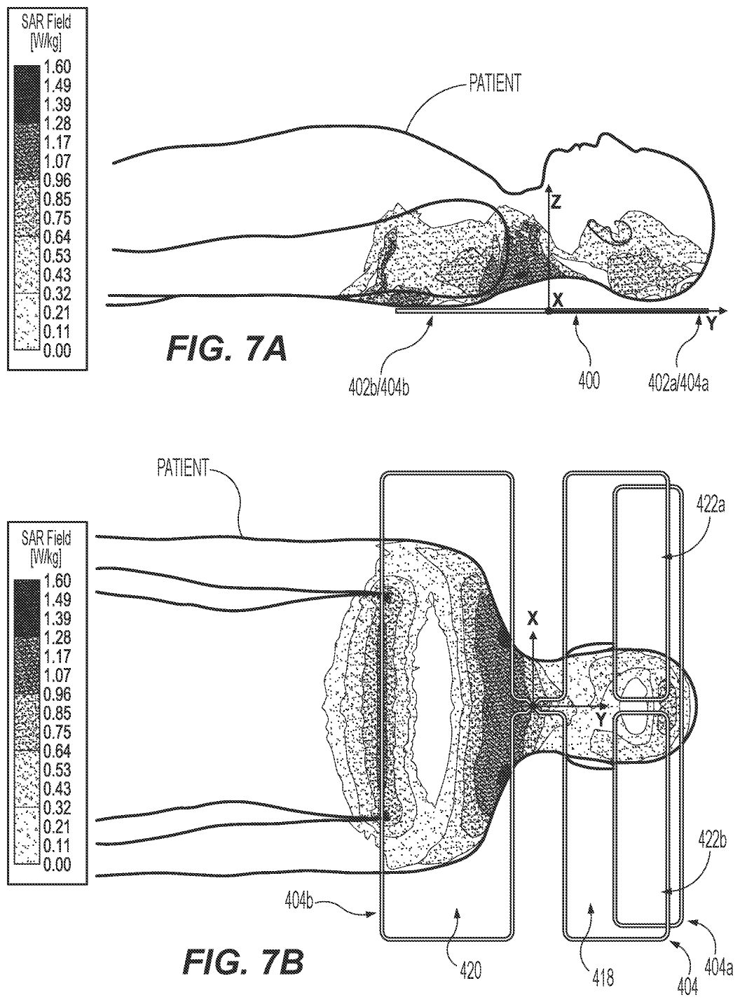

FIGS. 7A and 7B are sagittal and coronal views, respectively, of simulated SAR within a patient exposed to an electromagnetic field generated by the antenna 404. As shown in FIGS. 7A and 7B, the SAR experienced by a patient positioned within the electromagnetic field generated by the antenna 404 does not exceed the regulatory threshold of 1.6 W/kg. However, as previously noted with reference to FIG. 5, the magnetic field generated by the antenna 404 has a horizontal component with a sufficient magnitude at the implantable device antenna 502 to provide operational power to the implantable device. The asymmetrical shape of the antenna 404 enables such a balance between performance and safety. By coupling the first length 406 of conductive material to the second length 408 of conductive material in series with alternating current flow directions, the antenna 404 can create an electromagnetic field with the intended horizontal component. Moreover, the individual magnetic fields generated by the first subloop 422a and the second subloop 422b constructively interfere with the magnetic field generated by the first loop 418 to increase the intensity of the magnetic field and increase the volume of space proximate the external device 400 within which the horizontal component of the magnetic field has sufficient magnitude to deliver an intended power to the implantable device.

The magnetic field generated by the antenna 404 is greater in magnitude at distances closer to the conductive material of the antenna 404. Accordingly, the magnitude of the magnetic field is greater at the first portion 404a of the antenna 404 containing the first and second subloops 422a, 422b. By asymmetrically distributing conductive material and the intensity of the magnetic field towards the first portion 404a of the antenna 404, greater power can be delivered to the implantable device without exceeding a predetermined SAR limit. SAR is proportional to the electrical conductivity of the specific tissue absorbing the energy. The first portion 404a of the antenna 404 is configured to be positioned proximate a patient's head, which comprises a substantial amount of bone tissue. Bone tissue has a lower electrical conductivity than other soft tissues such as muscle or fat, and is therefore less susceptible to absorption of electromagnetic energy. Moreover, the head is smaller and rounder, and may be positioned on a pillow such that the head is further from the antenna 404. Thus, as shown in FIGS. 7A and 7B, the SAR at the patient's head remains below the regulatory threshold, despite the additional conductive material at the first portion 404a of the antenna 404. Further, as shown in FIGS. 7A and 7B, the SAR is highest in the regions proximate the patient's armpits, back, and neck, which are positioned directly over the conductive material of the second portion 404b of the antenna 404. The skin, muscle, and fat in these regions have greater electrical conductivity values and are therefore more susceptible to absorption of electromagnetic energy than the head. Accordingly, antennas 404 of the present technology can have a variable density of conductive material based at least in part on a conductivity of a patient tissue configured to be positioned proximate specific portions of the antenna 404. For example, the first portion 404a can be configured to be positioned proximate a head of a patient and can therefore have a higher density of conductive material than a second portion 404b that is configured to be positioned proximate a back of the patient.

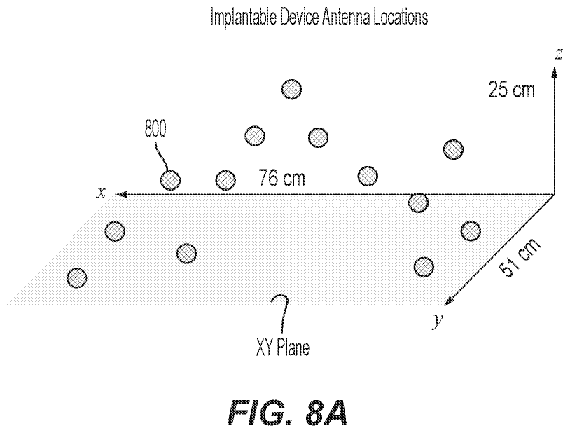

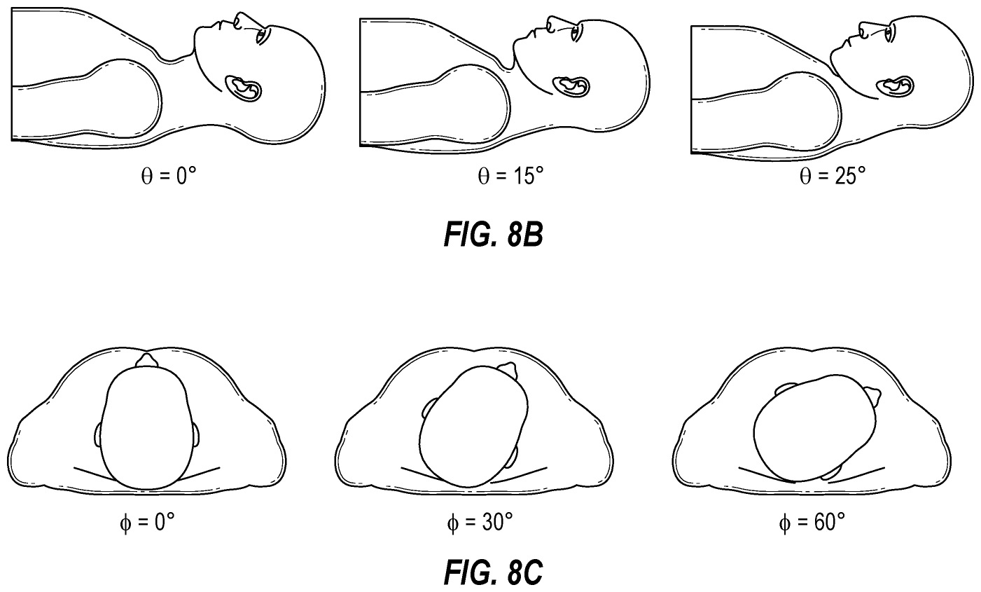

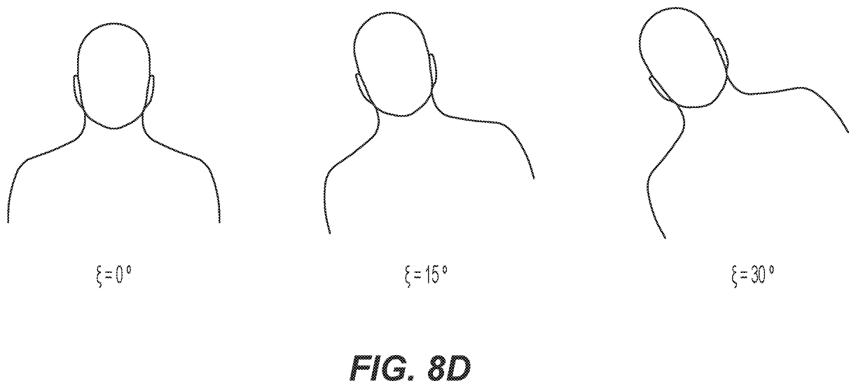

As previously noted, the antenna 404 can comprise conductive material in a specific shape such that the antenna 404 is configured to generate an electromagnetic field having a horizontal component at an expected position of the implantable device antenna with sufficient magnitude to induce an intended current in the implantable device antenna. However, the actual position of the implantable device antenna can vary with patient positioning relative to the external device 400, patient motion during sleep, etc. For example, FIG. 8A depicts a variety of positions of an implantable device antenna 800 (only one antenna 800 is labeled for ease of illustration). The implantable device antenna 800 may be located at such positions during normal use of a neuromodulation system of the present technology. As shown in FIG. 8A, the position of the implantable device antenna 800 can vary in three dimensions (e.g., in an x-direction, in a y-direction, in a z-direction, etc.). According to various embodiments, the x-direction shown in FIG. 8A can substantially correspond to the width dimension W of the antenna 404 and the y-direction shown in FIG. 8A can substantially correspond to the length dimension L of the antenna 404 (e.g., the antenna 404 can lie in an x-y plane defined by the x-direction and the y-direction). The antenna 404 can be configured to generate an electromagnetic field having a horizontal component that is configured to extend through an antenna of the implant in a direction substantially perpendicular to a radial dimension of the antenna of the implant across a range of head positions. For example, the antenna 404 can be configured to generate an electromagnetic field having an active volume encompassing a range of head positions in which a horizontal component of the electromagnetic field has sufficient magnitude to induce an intended current in an implantable device antenna positioned within the active volume. The active volume can be at least 25 cubic centimeters. In some embodiments, the active volume spans at least 20 cm in the x-direction, at least 50 cm in the y-direction, and/or at least 1 cm in the z-direction. According to various embodiments, the active volume is about 76 cm in the x-direction, about 51 cm in the y-direction, and/or about 25 cm in the z-direction.

As shown in FIGS. 8B-8D, a patient's head may be positioned at and/or move through a range of nod angles θ (FIG. 8B), axial head angles ϕ (FIG. 8C), and/or head rotation angles ξ (FIG. 8D). To power to the implant even if the patient moves during sleep, the antenna 404 can be configured to generate an electromagnetic field having a horizontal component that is configured to extend through an antenna of the implant in a direction substantially perpendicular to a radial dimension of the antenna of the implant across a range of nod angles θ, axial head angles ϕ, and/or head rotation angles. The antenna 404 can be configured to generate such an electromagnetic field when a patient's nod angle θ varies from about 0 degrees to about 30 degrees, when a patient's axial head angle ϕ varies from about −60 degrees to about 60 degrees, and/or when a patient's head rotation angle ξ varies from about −30 degrees to about 30 degrees.

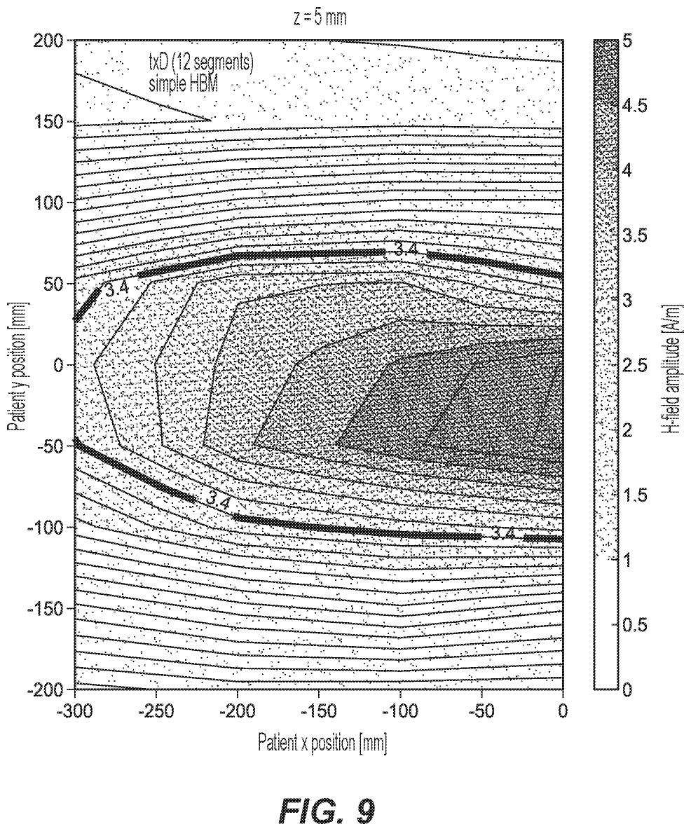

Performance of the antenna 404 of the external device 400 relates to the ability of the antenna 404 to provide operational power to the implantable device antenna, which may be located at a variety of positions over time, without exceeding regulatory exposure limits. FIG. 9 summarizes such performance of the antenna 404. Specifically, FIG. 9 is a contour plot of psSAR-limited average magnitude of the component of the magnetic field perpendicularly penetrating the implantable device antenna (H-field magnitude) over a variety of positions of a patient's chin relative to the antenna 404. FIG. 9 depicts the H-field magnitude for a given position of the patient's chin in the x-direction (e.g., along the width dimension W of the antenna 404) and in the y-direction (e.g., along the length dimension L of the antenna 404). At 0 mm in the x-direction and 0 mm in the y-direction, the patient's chin is located at the first ends 410, 414 and the second ends 412, 416 of the first and second lengths 406, 408, respectively. FIG. 9 depicts the H-field magnitude when the patient's chin is fixed at 5 mm from the antenna 404 in the height dimension perpendicular to the length dimension L and the width dimension W of the antenna 404. The H-field magnitude represents the maximum average magnitude of the component of the magnetic field perpendicularly penetrating the implantable device antenna that does not cause the psSAR within the patient to exceed the regulatory threshold of 1.6 W/kg.

For a neuromodulation system of the present technology, a minimum H-field magnitude can be obtained based on a power requirement of the implantable device. For example, a psSAR-limited H-field magnitude of about 3.4 A/m (2.4 A/m rms) can provide sufficient power to an implantable device to perform intended functions. Accordingly, the contour line labeled 3.4 A/m in FIG. 9 delineates patient positions at which the H-field magnitude is below 3.4 A/m (i.e., an inactive area) from patient positions at which the H-field magnitude exceeds 3.4 A/m (i.e., an active area). Patient positions within the active area are positions at which an implantable device receives sufficient power from an electromagnetic field generated by the antenna 404. As shown in FIG. 9, the H-field magnitude is greatest when the patient's chin is centered over the antenna 404 and decreases as the patient's chin is translated in the x-direction and/or the y-direction. A greater H-field magnitude indicates that, at that position, more power can be delivered to an implantable device before reaching the psSAR regulatory threshold.

As previously noted, FIG. 9 depicts the H-field magnitude when the patient's chin is fixed at 5 mm from the antenna 404 in the height dimension perpendicular to the length dimension L and the width dimension W. Generally, the active volume within which the H-field magnitude is sufficiently high to provide an intended power to the implantable device antenna without exceeding the SAR regulatory threshold increases as the distance from the patient's chin to the antenna 404 in the height dimension increases. This is because the magnitude of the magnetic field decreases rapidly with distance from the conductive material of the antenna 404, so even small separations along the height dimension can provide a significant SAR reduction. The magnitude of the magnetic field at the height of the implantable device antenna also decreases with distance from the antenna 404, but at a slower rate than at the height of the conductive material, so the magnitude remains sufficient to provide power to the implantable device antenna. Accordingly, in some embodiments the carrier 402 of the external device 400 has a portion configured to be positioned between the antenna 404 and the patient to define a minimum distance between the antenna 404 and the patient and increase the active volume size.

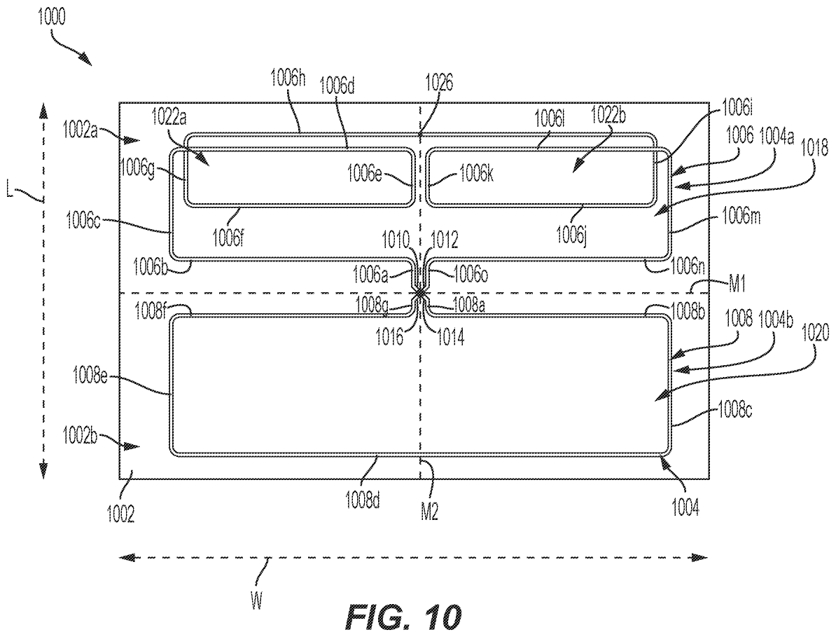

While FIG. 4A depicts the antenna 404 having a specific shape and specific dimensions, other configurations of the antenna 404 are within the scope of the present technology. According to various embodiments, a geometry of the conductive material, the distribution of capacitors, etc. can be selected based on an intended performance of the antenna. As but one example, FIG. 10 illustrates an external device 1000 comprising a carrier 1002 carrying an antenna 1004. The antenna 1004 can be similar to antenna 404. For example, the antenna 1004 can comprise conductive material in the same shape as the antenna 404. However, in contrast with the capacitors 428 shown in FIG. 4A, the antenna 1004 of FIG. 10 does not include capacitors coupled in series with the conductive material. The inclusion or the exclusion of the series capacitors may not substantially affect the magnitude or distribution of the magnetic field generated by the antenna 1004. Accordingly, the magnetic field and psSAR generated by the antenna 1204 can be substantially similar to those shown in FIGS. 5, 7A and 7B, respectively.

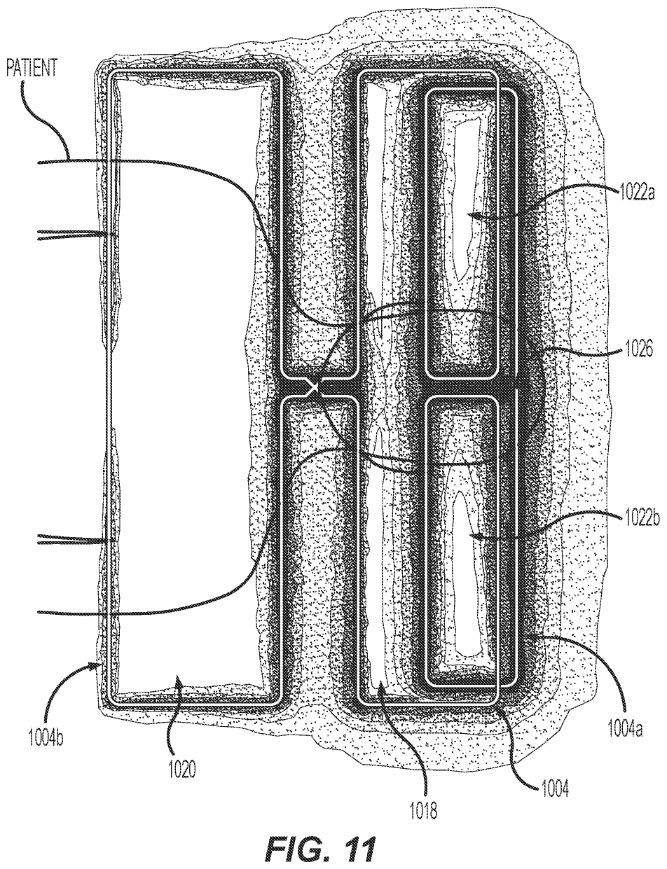

FIG. 11 is a coronal view of an intensity map of an electric field produced by the antenna 1004 in the plane of the antenna 1004. As shown in FIGS. 6 and 11, an electric field generated by the antenna 1004 without series capacitors (FIG. 11) is greater in a plane of the antenna 1004 than the electric field generated by the antenna 404 with series capacitors 428 (FIG. 6). As previously noted, the series capacitors 428 segment the conductive material of the antenna 404 and limit the peak voltage (and thereby the peak electric field) that can develop in the antenna 404. Without the series capacitors, the voltage of the antenna 1004 increases from an input terminal of the feed 1026 to an output terminal of the feed 1026. It can be useful to limit the peak voltage across an antenna of the present technology for patient safety.

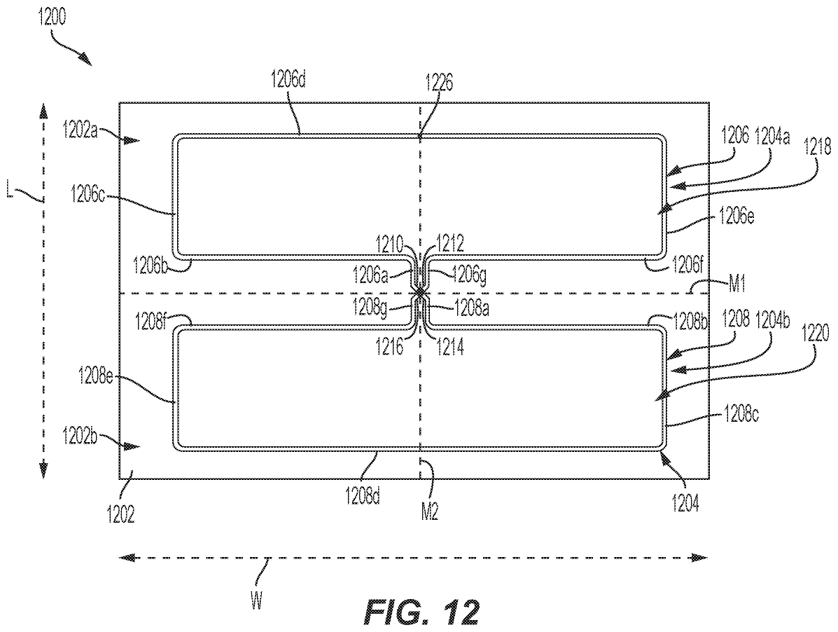

FIG. 12 depicts an external device 1200 comprising a carrier 1202 carrying an antenna 1204. Similar to the antenna 404, the antenna 1204 shown in FIG. 12 comprises a first portion 1204a including a first length 1206 of conductive material and a second portion 1204b opposite the first portion 1204a along a length dimension L of the carrier 1202 and including a second length 1208 of conductive material. However, as shown in FIG. 12, the antenna 1204 can be symmetric about the first midline M1. In these and other embodiments, the first length 1206 of conductive material can have a similar total length and/or the same total length as the second length 1208.

As shown in FIG. 12, the first length 1206 of conductive material can extend from a first end 1210 to a second end 1212 and/or the second length 1208 of conductive material can extend from a first end 1214 to a second end 1216. In some embodiments, the first length 1206 forms a first loop 1218 and/or the second length 1208 forms a second loop 1220. In some embodiments, the first length 1206 does not form subloops and/or the first loop 1218 encloses an area substantially equivalent to an area enclosed by the second loop 1220. The first length 1206 of conductive material and the second length 1208 of conductive material can be electrically coupled to one another in series (e.g., via connection of the first ends 1210, 1214 and connection of the second ends 1212, 216).

As shown in FIG. 12, the first length 1206 can comprise a first region 1206a extending from a first end of the first region 1206a at the first end 1210 of the first length 1206 to a second end of the first region 1206a in the first length direction, a second region 1206b extending from a first end of the second region 1206b at the second end of the first region 1206a to a second end of the second region 1206b in the first width direction, a third region 1206c extending from a first end of the third region 1206c at the second end of the second region 1206b to a second end of the third region 1206c in the first length direction, a fourth region 1206d extending from a first end of the fourth region 1206d at the second end of the third region 1206c to a second end of the fourth region 1206d in the second width direction, a fifth region 1206e extending from a first end of the fifth region 1206e at the second end of the fourth region 1206d to a second end of the fifth region 1206e in the second length direction, a sixth region 1206f extending from a first end of the sixth region 1206f at the second end of the fifth region 1206e to a second end of the sixth region 1206f in the first width direction, and a seventh region 1206g extending from a first end of the seventh region 1206g at the second end of the sixth region 1206f to a second end of the seventh region 1206g at the second end 1212 of the first length 1206 in the second length direction. Collectively, the first-seventh regions 1206a-1206g of the first length 1206 form the first loop 1218.

The second length 1208 can comprise a first region 1208a extending from a first end of the first region 1208a at the first end 1214 of the second length 1208 to a second end of the first region 1208a in the second length direction, a second region 1208b extending from a first end of the second region 1208b at the second end of the first region 1208a to a second end of the second region 1208b in the second width direction, a third region 1208c extending from a first end of the third region 1208c at the second end of the second region 1208b to a second end of the third region 1208c in the second length direction, a fourth region 1208d extending from a first end of the fourth region 1208d at the second end of the third region 1208c to a second end of the fourth region 1208d in the first width direction, a fifth region 1208e extending from a first end of the fifth region 1208e at the second end of the fourth region 1208d to a second end of the fifth region 1208e in the first length direction, a sixth region 1208f extending from a first end of the sixth region 1208f at the second end of the fifth region 1208e to a second end of the sixth region 1208f in the second width direction, and a seventh region 1208g extending from a first end of the seventh region 1208g at the second end of the sixth region 1208f to a second end of the seventh region 1208g at the second end 1216 of the second length 1208 in the first length direction. Collectively, the first-seventh regions 1208a-1208g of the second length 1208 form the second loop 1220.

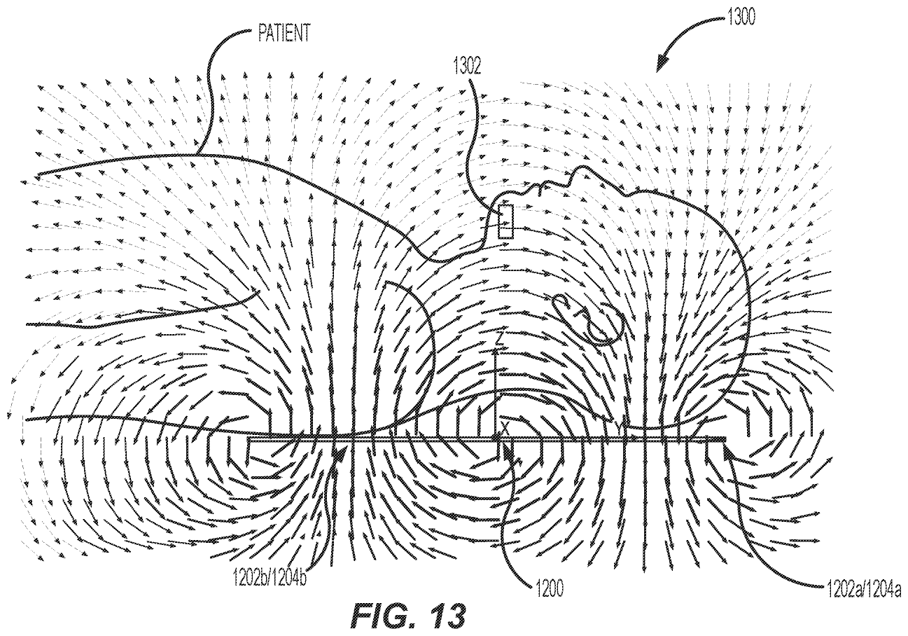

FIG. 13 is a two-dimensional view of a magnetic field 1300 generated by the antenna 1204. Similar to the magnetic field 500 generated by the antenna 404, the magnetic field 1300 shown in FIG. 13 can have a horizontal component that is substantially parallel to a plane containing the first length 1206 and the second length 1208 of the antenna 1204 (e.g., defined by the width and length dimensions of the antenna 1204, substantially parallel to a broad surface of the carrier 1202, etc.). When the external device 1200 is positioned between a patient and a surface with an implantable device antenna 1302 angled with respect to the plane containing the first length 1206 and the second length 1208, the horizontal component of the magnetic field 1300 can be substantially perpendicular to the implantable device antenna 1302 and can induce an electromotive force in the implantable device antenna 1302. However, in contrast to the magnetic field 500 generated by the antenna 404, the magnetic field 1300 generated by the antenna 1204 of FIG. 12 may be substantially symmetric about the first midline M1 of the carrier 1202.

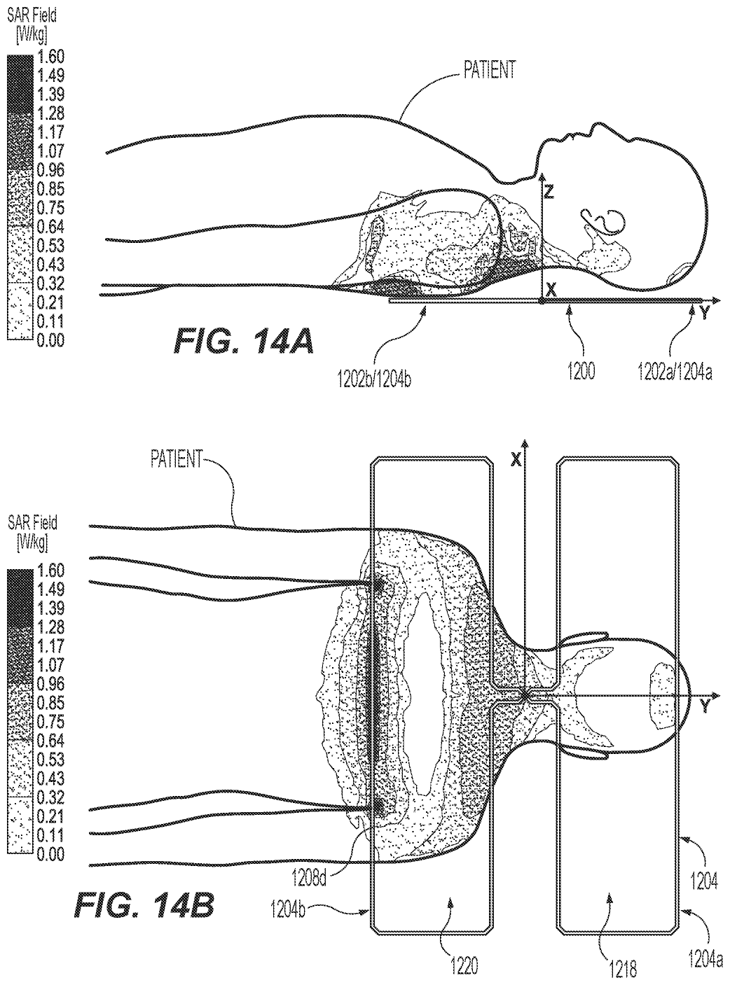

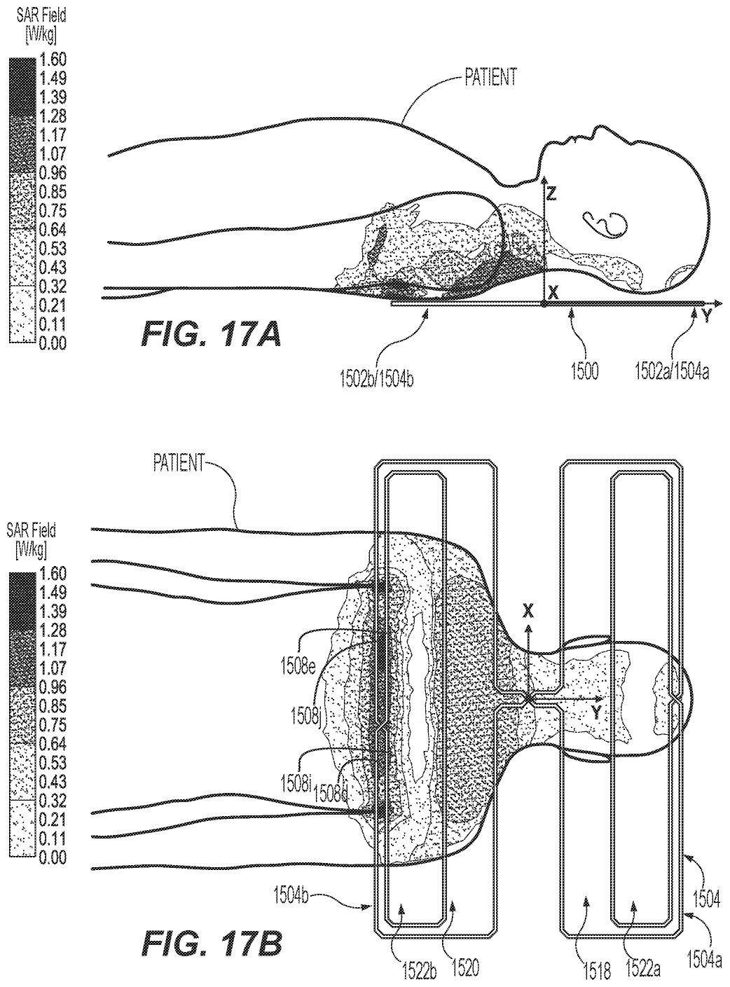

As shown in FIG. 13, the magnetic field 1300 generated by the antenna 1204 can have a generally lower intensity than the magnetic field 500 generated by the antenna 404 in response to the same excitation energy. The lower intensity of the magnetic field 1300 results from the reduced number of loops and lower density of conductive material of the antenna 1204. Such changes in magnetic field intensity can also cause changes in the SAR that develops from exposure to the magnetic field 1300. For example, as shown in FIGS. 14A and 14B, SAR developed in a head of a patient in response to the magnetic field 1300 can be lower than the SAR developed in the head of the patient in response to the magnetic field 500. Such relative decrease in SAR can be attributed to the lower intensity of the magnetic field 1300 at the first portion 1204a of the antenna 1204. Additionally, as shown in FIGS. 17A and 17B, SAR may be greater in regions with higher magnetic field 1300 intensity and higher electrical conductivity of the tissue. For example, peak SAR occurs approximately at the level of the armpits near the fourth region 1208d of the second length 1208 of the antenna 1204. Generally, SAR tends to be greatest at anatomical regions positioned directly over or near the first and second lengths 1206, 1208 of conductive material.

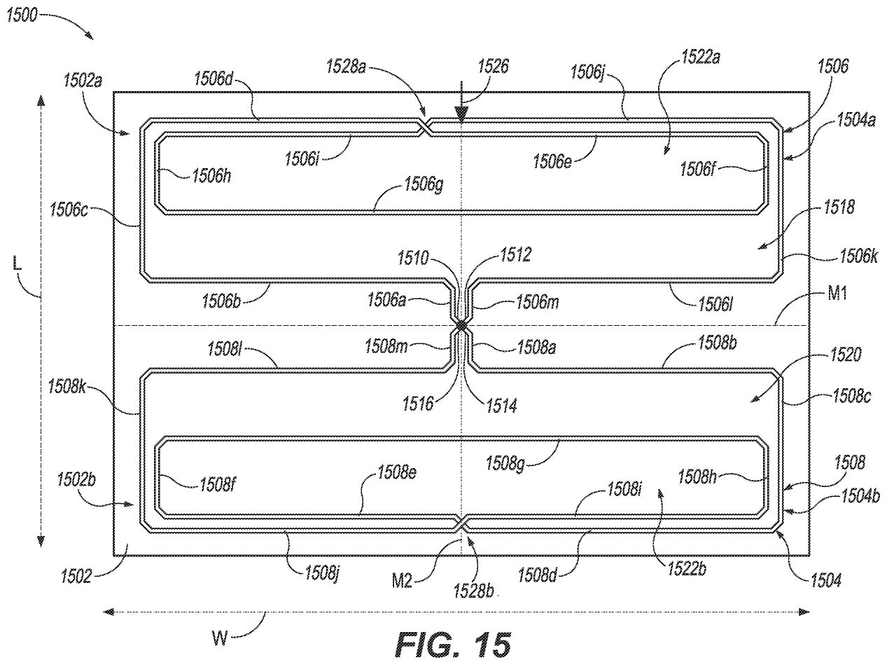

FIG. 15 depicts an external device 1500 comprising a carrier 1502 carrying an antenna 1504 comprising a first portion 1504a including a first length 1506 of conductive material and a second portion 1504b opposite the first portion 1504a along a length dimension L of the carrier 1502 and including a second length 1508 of conductive material. Similar to the antenna 1204, the antenna 1504 shown in FIG. 15 can be symmetric about the first midline M1. For example, the first length 1506 can form a first loop 1518 and the second length 1508 can form a second loop 1520. However, in contrast to the antenna 1204 the first length 1506 can form a first subloop 1522a and the second length 1508 can form a second subloop 1522b. The first subloop 1522a and/or the second subloop 1522b can enclose a smaller area than the first loop 1518 and/or the second loop 1520.

As shown in FIG. 15, the first length 1506 can comprise a first region 1506a extending from a first end of the first region 1506a at the first end 1510 of the first length 1506 to a second end of the first region 1506a in the first length direction, a second region 1506b extending from a first end of the second region 1506b at the second end of the first region 1506a to a second end of the second region 1506b in the first width direction, a third region 1506c extending from a first end of the third region 1506c at the second end of the second region 1506b to a second end of the third region 1506c in the first length direction, and a fourth region 1506d extending from a first end of the fourth region 1506d at the second end of the third region 1506c to a second end of the fourth region 1506d in the second width direction. The fourth region 1506d can span a similar distance along the width dimension W as the second region 1506b or can span a different distance along the width dimension W as the second region 1506b.

The first length 1506 can comprise a fifth region 1506e extending from a first end of the fifth region 1506e to a second end of the fifth region 1506e in the second width direction. As shown in FIG. 15, in some embodiments the first length 1506 comprises a first transition region 1528a at which the first length 1506 transitions from the first loop 1518 to the first subloop 1522a. At the first transition region 1528a, the first length 1506 extends diagonally relative to the length dimension L and the width dimension W between the second end of the fourth region 1506d and the first end of the fifth region 1506e so that the second end of the fourth region 1506d and the first end of the fifth region 1506e are offset along the width dimension W and the length dimension L. The first length 1506 can comprise a sixth region 1506f extending from a first end of the sixth region 1506f at the second end of the fifth region 1506e to a second end of the sixth region 1506f in the second length direction, a seventh region 1506g extending from a first end of the seventh region 1506g at the second end of the sixth region 1506f to a second end of the seventh region 1506g in the first width direction, an eighth region 1506h extending from a first end of the eighth region 1506h at the second end of the seventh region 1506g to a second end of the eighth region 1506h in the first length direction, and a ninth region 1506i extending from a first end of the ninth region 1506i at the second end of the eighth region 1506h to a second end of the ninth region 1506i in the second width direction. Collectively, the fifth-ninth regions 1506e-1506i of the first length 1506 can form the first subloop 1522a.

The first length 1506 can comprise a tenth region 1506j extending from a first end of the tenth region 1506j to a second end of the tenth region 1506j in the second width direction. As shown in FIG. 15, in some embodiments the first length 1506 extends diagonally relative to the length dimension L and the width dimension W between the second end of the ninth region 1506i and the first end of the tenth region 1506j at the first transition region 1528a. Thus, the second end of the ninth region 1506i and the first end of the tenth region 1506j can be offset along the width dimension W and the length dimension L. The first length 1506 can comprise an eleventh region 1506k extending from a first end of the eleventh region 1506k at the second end of the tenth region 1506j to a second end of the eleventh region 1506k in the second length direction, a twelfth region 1506l extending from a first end of the twelfth region 1506l at the second end of the eleventh region 1506k to a second end of the twelfth region 1506l in the first width direction, and a thirteenth region 1506m extending from a first end of the thirteenth region 1506m at the second end of the twelfth region 1506l to a second end of the thirteenth region 1506m at the second end 1512 of the first length 1506 in the second length direction. Collectively, the first-fourth regions 1506a-1506d and the tenth-thirteenth regions 1506j-1506m of the first length 1506 can form the first loop 1518. According to various embodiments, an area enclosed by the first loop 1518 can be greater than an area enclosed by the first subloop 1522a.

As shown in FIG. 15, the second length 1508 can comprise a first region 1508a extending from a first end of the first region 1508a at the first end 1514 of the second length 1508 to a second end of the first region 1508a in the second length direction, a second region 1508b extending from a first end of the second region 1508b at the second end of the first region 1508a to a second end of the second region 1508b in the second width direction, a third region 1508c extending from a first end of the third region 1508c at the second end of the second region 1508b to a second end of the third region 1508c in the second length direction, and a fourth region 1508d extending from a first end of the fourth region 1508d at the second end of the third region 1508c to a second end of the fourth region 1508d in the first width direction. The fourth region 1508d can span a similar distance along the width dimension W as the second region 1508b or can span a different distance along the width dimension W as the second region 1508b.

The second length 1508 can comprise a fifth region 1508e extending from a first end of the fifth region 1508e to a second end of the fifth region 1508e in the first width direction. As shown in FIG. 15, in some embodiments the second length 1508 comprises a second transition region 1528b at which the second length 1508 transitions from the second loop 1520 to the second subloop 1522b. At the second transition region 1528b, the second length 1508 extends diagonally relative to the length dimension L and the width dimension W between the second end of the fourth region 1508d and the first end of the fifth region 1508e so that the second end of the fourth region 1506d and the first end of the fifth region 1508e are offset along the width dimension W and the length dimension L. The second length 1508 can comprise a sixth region 1508f extending from a first end of the sixth region 1508f at the second end of the fifth region 1508e to a second end of the sixth region 1508f in the first length direction, a seventh region 1508g extending from a first end of the seventh region 1508g at the second end of the sixth region 1508f to a second end of the seventh region 1508g in the second width direction, an eighth region 1508h extending from a first end of the eighth region 1508h at the second end of the seventh region 1508g to a second end of the eighth region 1508h in the second length direction, and a ninth region 1508i extending from a first end of the ninth region 1508i at the second end of the eighth region 1508h to a second end of the ninth region 1508i in the first width direction. Collectively, the fifth-ninth regions 1508e-1508i of the second length 1508 can form the second subloop 1522b.

The second length 1508 can comprise a tenth region 1508j extending from a first end of the tenth region 1508j to a second end of the tenth region 1508j in the first width direction. As shown in FIG. 15, in some embodiments the second length 1508 extends diagonally relative to the length dimension L and the width dimension W between the second end of the ninth region 1508i and the first end of the tenth region 1508j at the second transition region 1528b. Thus, the second end of the ninth region 1508i and the first end of the tenth region 1508j can be offset along the width dimension W and the length dimension L. The second length 1508 can comprise an eleventh region 1508k extending from a first end of the eleventh region 1508k at the second end of the tenth region 1508j to a second end of the eleventh region 1508k in the first length direction, a twelfth region 1508l extending from a first end of the twelfth region 1508l at the second end of the eleventh region 1508k to a second end of the twelfth region 1508l in the second width direction, and a thirteenth region 1508m extending from a first end of the thirteenth region 1508m at the second end of the twelfth region 1508l to a second end of the thirteenth region 1508m at the second end 1516 of the second length 1508 in the first length direction. Collectively, the first-fourth regions 1508a-1508d and the tenth-thirteenth regions 1508j-1508m of the second length 1508 can form the second loop 1520. According to various embodiments, an area enclosed by the second loop 1520 can be greater than an area enclosed by the second subloop 1522b.

As described with reference to the feed 426 of FIG. 4A, the antenna 1504 shown in FIG. 15 can include a feed 1526 at which the antenna 1504 receives current from a control unit and delivers current to a control unit. Although FIG. 15 illustrates the feed 1526 located substantially at the second midline M2, the feed 1526 can be located at any suitable location of the antenna 1504. Although a single feed 1526 is shown in FIG. 15, in various embodiments the antenna 1504 can include multiple feeds 1526 or no feed 1526, as described elsewhere herein.

The first transition region 1528a and/or the second transition region 1528b can be positioned at either side of the second midline M2 along the width dimension W and/or can be aligned with the second midline M2. The symmetry of the antenna 1504 about the second midline M2 can influence the electronics at the control unit that provide electrical energy to the antenna 1504 (e.g., via feed 1526). For example, in various embodiments the control unit includes a balanced amplifier for driving the antenna. Accordingly, it can be advantageous for an antenna of the present technology to be substantially symmetric about the second midline M2 to prevent or limit stray current from developing in the antenna and/or reaching the control unit. In some embodiments, such symmetry can be obtained by positioning the transition regions 1528a, 1528b near or at the second midline M2. As shown in FIG. 15, in some embodiments the feed 1526 can be positioned at the second midline M2, so the first transition region 1528a can be positioned near but not directly at the second midline M2.

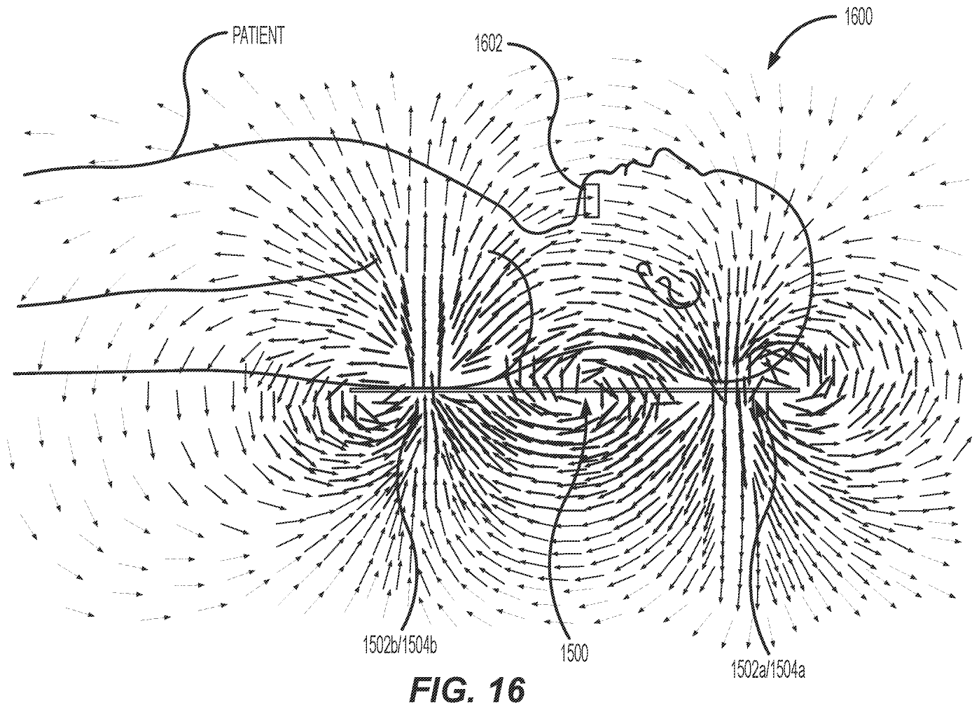

As shown in FIG. 16, a magnetic field 1600 generated by the antenna 1504 can have a greater intensity than the magnetic field 1300 generated by the antenna 1204, which can facilitate greater power transfer from the antenna 1504 to an antenna of an implantable device 1602. The inclusion of the subloops 1522a, 1522b increases an intensity of the magnetic field relative to the individual first and second loops 1218, 1220 of the antenna 1204 of FIG. 12. The individual magnetic fields generated by each of the first loop 1518 and the first subloop 1522a can at least partially constructively interfere so that an intensity of a resultant magnetic field is greater than an intensity of the individual magnetic field generated by either of the first loop 1518 or the first subloop 1522a. Similarly, the magnetic fields generated by each of the second loop 1520 and the second subloop 1522b can at least partially constructively interfere so that an intensity of a resultant magnetic field is greater than an intensity of the individual magnetic field generated by either of the second loop 1520 or the second subloop 1522b.

FIGS. 17A and 17B are sagittal and coronal views, respectively, of simulated SAR within a patient exposed to the magnetic field 1600 generated by the antenna 1504. The SAR is generally greater at anatomical regions positioned within portions of the magnetic field 1600 having greater intensity. Moreover, as discussed herein, the head comprises a substantial amount of bone tissue, which has a lower electrical conductivity and greater robustness to electromagnetic radiation exposure than other soft tissues. Thus, the SAR at the patient's head in FIGS. 17A and 17B may not be significantly greater than the SAR at the patient's head in FIGS. 14A and 14B, despite the intensity of the magnetic field 1600 at the head being greater than the intensity of the magnetic field 1300 at the head. However, the soft tissues of the neck and upper back (e.g., muscle, fat, etc.) are more susceptible to increases in SAR. As shown in FIGS. 17A and 17B, the magnetic field 1600 generated by the antenna 1504 can substantially increase the SAR at the patient's shoulders, armpits, neck, and/or upper back relative to the magnetic field 1300 generated by the antenna 1204. As the magnitude of the magnetic field is greatest near the conductive material of the antenna 1504, the additional density of conductive material at the second portion 1504b of the antenna 1504 increases the SAR in the upper back of the patient. Thus, it may be advantageous for an antenna of the present technology to have a lower density of conductive material at regions of the carrier that are configured to be positioned proximate an anatomical region having a higher electrical conductivity to prevent excessive SAR absorption in the patient.

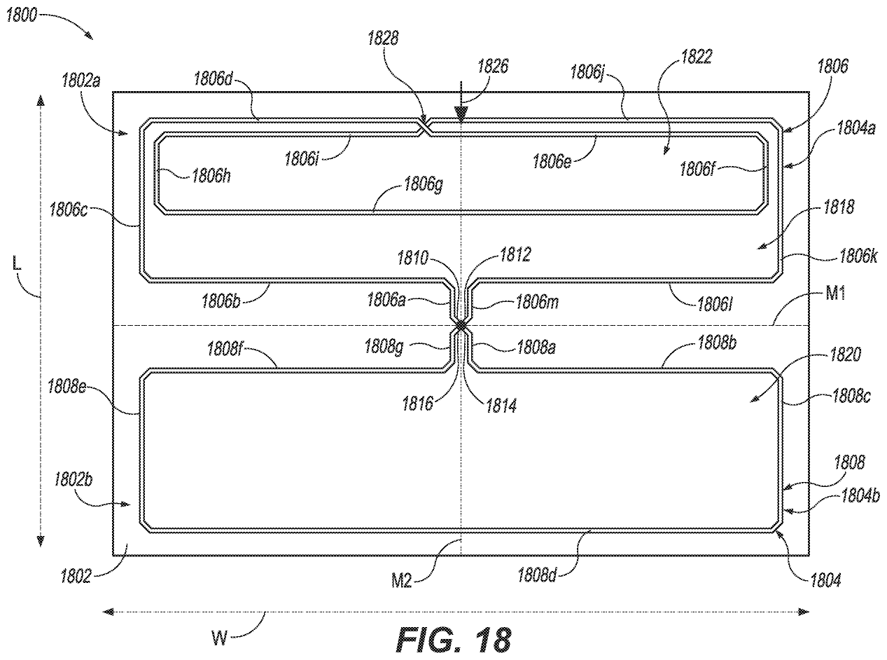

FIG. 18 depicts an example of an external device 1800 comprising a carrier 1802 carrying an antenna 1804 having a second portion 1804b with a lower density of conductive material at a second region 1802b of the carrier 1802, which is configured to be positioned proximate a back and/or neck of a patient. A first portion 1804a of the antenna 1804 having a higher density of conductive material and positioned at a first region 1802a of the carrier 1802 is configured to be positioned proximate a head of the patient. The first portion 1804a can comprise a first length 1806 of conductive material extending from a first end 1810 to a second end 1812 and the second portion 1804b can comprise a second length 1808 of conductive material extending from a first end 1814 to a second end 1816. The first length 1806 can comprise a greater total length than the second length 1808. The first length 1806 can form a first loop 1818 and the second length 1808 can form a second loop 1820. As shown in FIG. 18, the first length 1806 can also form a subloop 1822. In contrast to the antenna 404 of FIG. 4A, the first length 1806 can form one subloop 1822 extending along the width dimension W instead of two subloops 422a, 422b spaced apart along the width dimension W.

The antenna 1804 can be configured to generate a magnetic field having a similar magnitude and distribution to the antenna 404. However, a magnetic field generated by the antenna 1804 of FIG. 18 may have a greater magnitude at the second midline M2 as compared to a magnetic field generated by the antenna 404 of FIG. 4A. Because current is configured to flow in opposite directions through the fifth region 406e of the first length 406 of the antenna 404 and the eleventh region 406k of the first length 406 of the antenna 404, the magnetic fields generated about the fifth region 406e and the eleventh region 406k may at least partially cancel each other such that an intensity of the magnetic field is lower at or near the second midline M2. However, because the first length 1806 of the antenna 1804 of FIG. 18 does not include such adjacent regions of the first length 1806 with current flowing in opposing directions, the magnetic field generated by the antenna 1804 can be greater at or near the second midline M2 than the magnetic field generated by the antenna 404.

As shown in FIG. 18, in some embodiments a transition region 1828 between the first loop 1818 and the subloop 1822 can be positioned at one side of the second midline M2 along the width dimension W. The symmetry of the antenna 1804 about the second midline M2 can influence the electronics at the control unit that provide electrical energy to the antenna 1804 (e.g., via feed 1826). For example, in various embodiments the control unit includes a balanced amplifier for driving the antenna. Accordingly, it can be advantageous for an antenna of the present technology to be substantially symmetric about the second midline M2 to prevent or limit stray current from developing in the antenna and/or reaching the control unit. In some embodiments, such symmetry can be obtained by positioning the transition region 1828 near or at the second midline M2.

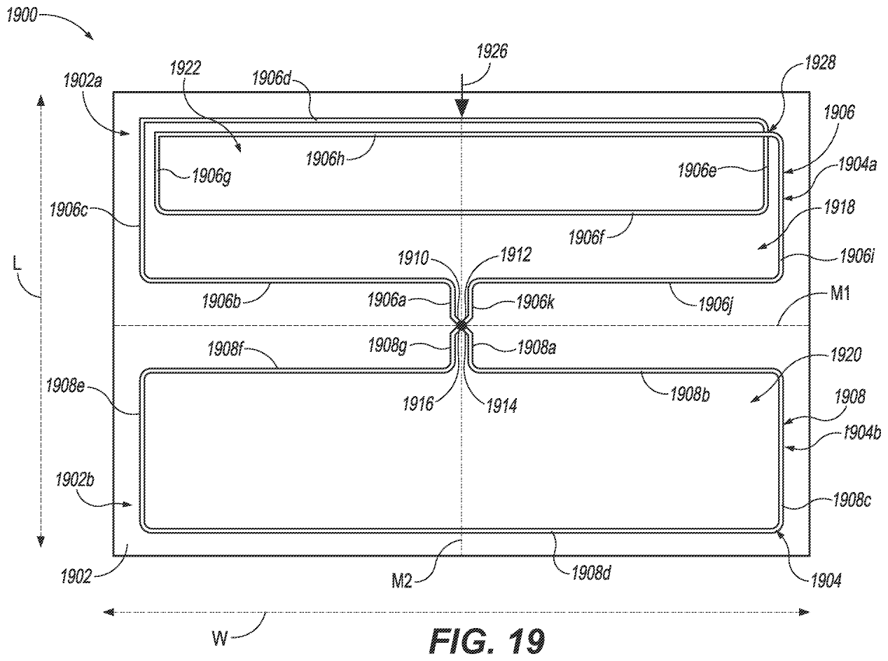

In some embodiments, a transition region between loops and/or subloops can be spaced apart from the second midline M2. FIG. 19 depicts an example of an antenna 1904 that is similar to the antenna 1804 of FIG. 18. For example, the antenna 1904 can comprise a first length 1906 of conductive material forming the first loop 1918 and the subloop 1922 and a second length 1908 of conductive material forming the second loop 1920. The first length 1906 can comprise first-eleventh regions 1906a-1906k extending sequentially between a first end 1910 of the first length 1906 and a second end 1912 of the first length 1906. The second length 1908 can comprise first-seventh regions 1908a-1908g extending sequentially between a first end 1914 of the second length 1908 and a second end 1916 of the second length 1908. Thus, the first length 1906 can have a greater total length than the second length 1908. However, a transition region 1928 between the first loop 1918 and the subloop 1922 is positioned away from the second midline M2 along the width dimension W.

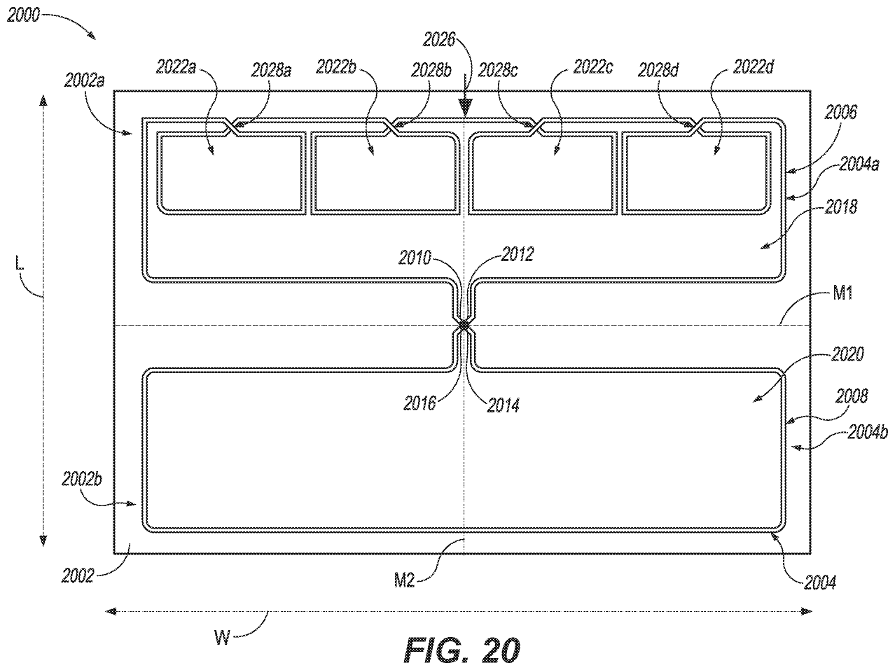

FIG. 20 depicts an external device 2000 comprising a carrier 2002 carrying an antenna 2004 comprising multiple subloops. Like the antennas previously described, the antenna 2004 shown in FIG. 20 comprises a first portion 2004a including a first length 2006 of conductive material and a second portion 2004b opposite the first portion 2004a along a length dimension L of the carrier 2002 and including a second length 2008 of conductive material. As shown in FIG. 20, the antenna 2004 can be asymmetric about the first midline M1 and/or symmetric about the second midline M2. In various embodiments, a feed 2026 of the antenna 2004 is located substantially at the second midline M2.

The first length 2006 of conductive material can extend from a first end 2010 to a second end 2012 and/or the second length 2008 of conductive material can extend from a first end 2014 to a second end 2016. The first length 2006 of conductive material and the second length 2008 of conductive material can be electrically coupled to one another in series (e.g., via connection of the first ends 2010, 2014 to one another and connection of the second ends 2012, 2016 to one another). The first length 2006 of conductive material can form a first loop 2018 and the second length 2008 of conductive material can form a second loop 2020. Moreover, as shown in FIG. 20, the first length 2006 of conductive material can form a first subloop 2022a, a second subloop 2022b, a third subloop 2022c, and/or a fourth subloop 2022d (collectively “subloops 2022”). In some embodiments, the first length 2006 and/or the second length 2008 can form one subloop 2022, multiple subloops 2022, or no subloops 2022. Some or all of the subloops 2022 can be connected to the first loop 2018 and/or adjacent ones of the subloops 2022 in series. In some embodiments, one, some, or all of the subloops 2022 can be fed independently such that the one, some, or all of the subloops 2022 are driven independently and/or have their own current. One, some, or all of the subloops 2022 can be passive such that current is not provided to the subloop(s) via a feed or via series connection with the first or second loops 2018, 2020, but current is induced in the subloop(s) due to their presence within a magnetic field generated by the antenna 2004. Some or all of the subloops 2022 can enclose the same area. Additionally or alternatively, some of all of the subloops 2022 can be spaced apart from the first midline M1 along the length dimension L by the same distance.

According to various embodiments, RF current can flow through the first loop 2018 and the second loop 2020 in opposing directions. RF current can flow through one, some, or none of the subloops 2022 in the same direction as the first loop 2018. As shown in FIG. 20, in some embodiments each of the subloops 2022 is continuous with the first loop 2018 via transition regions 2028a-2028d. The transition regions 2028a-2028d can be similar to any other transition regions disclosed herein and/or any of the transition regions disclosed herein can have similar features as the transitions regions 2028a-2028d. At each of the transition regions 2028a-2028d the first length 2006 can cross over itself to provide an electrical path for current to flow between regions of the first length 2006 forming the first loop 2018 and regions of the first length 2006 forming the respective subloop 2022.

Because the antenna 2004 comprises discrete subloops 2022 spaced apart along the width dimension W, an intensity of the magnetic field generated by the antenna 2004 can vary along the width dimension W. When current flows through each of the subloops 2022 in the same direction, current flows through regions of the first length 2006 that are adjacent along the width dimension W (e.g., regions extending along the length dimension L) in opposite directions. Accordingly, the magnetic fields generated by current flowing through such regions can destructively interfere. In various embodiments, such destructive interference can limit an intensity of the magnetic field along the width dimension W. In contrast, the larger subloop at the first portion of each of the antennas 1504, 1804, 1904 of FIGS. 15, 18 and 19 can produce a magnetic field with a greater intensity at the second midline M2. In some embodiments, an intensity of the magnetic field and/or one or more components thereof can be relatively constant or change only to a small degree across the width dimension W.

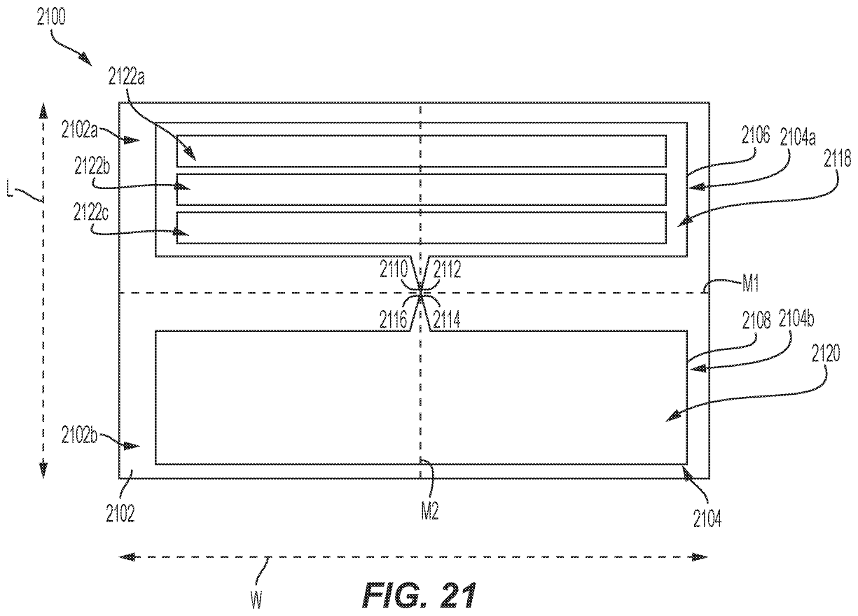

FIG. 21 depicts an external device 2100 comprising a carrier 2102 carrying an antenna 2104 comprising multiple subloops. Like the antennas previously described, the antenna 2104 shown in FIG. 21 comprises a first portion 2104a including a first length 2106 of conductive material and a second portion 2104b opposite the first portion 2104a along a length dimension L of the carrier 2102 and including a second length 2108 of conductive material. The first length 2106 of conductive material can extend from a first end 2110 to a second end 2112 and/or the second length 2108 of conductive material can extend from a first end 2114 to a second end 2116. The first length 2106 of conductive material can form a first loop 2118 and the second length 2108 of conductive material can form a second loop 2120. Moreover, as shown in FIG. 21, the first length 2106 of conductive material can form two or more subloops 2122 (e.g., two subloops 2122, three subloops 2122, four subloops 2122, etc.). As shown in FIG. 21, in some embodiments the first length 2106 forms a first subloop 2122a, a second subloop 2122b, and/or a third subloop 2122c. In contrast to the antennas previously described, the subloops 2122 shown in FIG. 21 can be spaced apart along the length dimension L.

The first length 2106 of conductive material and the second length 2108 of conductive material can be electrically coupled to one another in series (e.g., via connection of the first ends 2110, 2114 to one another and connection of the second ends 2112, 2116 to one another). Some or all of the subloops 2122 can be connected to the first loop 2118 and/or adjacent ones of the subloops 2122 in series. In some embodiments, one, some, or all of the subloops 2122 can be fed independently such that the one, some, or all of the 2122 are driven independently and/or have their own current. One, some, or all of the subloops 2122 can be passive such that current is not provided to the subloop via a feed but the subloop carries current by virtue of presence of the subloop within a magnetic field generated by the antenna 2104. Some or all of the subloops 2122 can enclose the same area. Additionally or alternatively, some of all of the subloops 2122 can be substantially aligned along the width dimension W of the carrier 2102. As shown in FIG. 21, the antenna 2104 can be asymmetric about the first midline M1 and/or symmetric about the second midline M2.

According to various embodiments, current can flow through the first loop 2118 and the second loop 2120 in opposing directions. Current can flow through one, some, or none of the subloops 2122 in the same direction as the first loop 2118. In some embodiments, current can flow through adjacent ones of the subloops 2122 in opposing directions. Although not shown in FIG. 21, in some embodiments one, some, or all of the subloops 2122 are continuous with the first loop 2118 via one or more transitions regions, which can be similar to any other transition regions disclosed herein. In some embodiments, the first length 2106 may include additional regions beyond those shown in FIG. 21 to facilitate transitions between subloops 2122, the first loop 2118, and/or the second loop 2120.

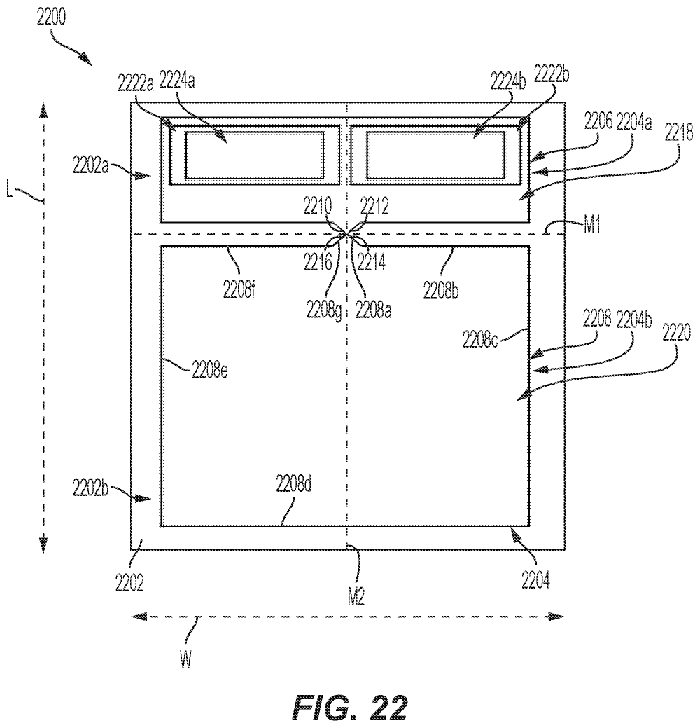

In some embodiments, a size of a second loop of an antenna can be modified based on an intended magnetic field to be produced by the antenna and/or a SAR threshold. For example, FIG. 22 depicts an external device 2200 comprising a carrier 2202 carrying an antenna 2204 having a first loop 2218 and a second loop 2220 with a larger area than the first loop 2218 and/or the second loops previously shown. Like the antennas previously described, the antenna 2204 shown in FIG. 22 comprises a first portion 2204a including a first length 2206 of conductive material and a second portion 2204b opposite the first portion 2204a along a length dimension L of the carrier 2202 and including a second length 2208 of conductive material. The first length 2206 of conductive material can form a first loop 2218 and the second length 2208 of conductive material can form a second loop 2220. Optionally, the first length 2206 can form one or more subloops 2222 (e.g., first subloop 2222a, second subloop 2222b, etc.). In some embodiments, the first length 2206 can form one or more secondary subloops 2224 (e.g., first secondary subloop 2224a, second secondary subloop 2224b, etc.). In some embodiments, the secondary subloops 2224 are nested within the subloops 2222 and/or enclose a smaller area than the subloops 2222.

An area of the second loop 2220 can be based on a width of the second loop 2220 along the width dimension W and/or a length of the second loop 2220 along the length dimension L. In some embodiments, the width and/or length of the second loop 2220 can be based on an intended positioning of the external device 2200 relative to one or more anatomical regions of a patient. For example, the length of the second loop 2220 can be selected such that when the first portion 2204a of the antenna 2204 is positioned proximate a patient's head, one or more regions of the second length 2208 are positioned proximate an anatomical region having a lower conductivity and therefore less susceptible to SAR. For example, a fourth region 2208d of the second length 2208 can be spaced apart from second and sixth regions 2208b, 2208f of the second length 2208 by a greater distance so that the fourth region 2208d is positioned more inferiorly relative to the patient when the external device 2200 is between the patient and the surface on which the patient lies. In this example, and others, the fourth region 2208d can be configured to align with an anatomical region of the patient having a lower electrical conductivity and less susceptibility to SAR absorption than the armpits and chest, such as the hips, for example.

In some embodiments, an area of the second loop 2220 can be at least partially based on an intended magnetic moment of the antenna 2204. It can be advantageous for a quadrupole magnetic moment of the antenna 2204 to be much larger than a dipole magnetic moment of the antenna 2204 so that radiation of the magnetic field at far distances is limited. The magnetic moments of the antenna 2204 can be at least partially based on a symmetry of the antenna 2204 in the length dimension L (e.g., an area enclosed by the first loop 2218 and an area enclosed by each of the subloops 2222 compared to an area enclosed by the second loop 2220, distances between certain regions of the first length 2206 and the first midline M1 compared to distances between certain regions of the second length 2208 and the first midline M1, etc.). If the first portion 2204a of the antenna 2204 has a greater density of conductive material than the second portion 2204b of the antenna 2204, it may be advantageous for a length and/or a width of the second loop 2220 to be greater than a corresponding length and/or width of the first loop 2218 and/or one or more of the subloops 2222. In some embodiments, the antenna 2204 can be symmetric about a midline extending along the width dimension W (e.g., the second midline M2).

An area of the first loop 2218, the subloops 2222, and/or the secondary subloops 2224 can be at least partially based on an intended magnetic moment of the antenna 2204. For example, if the area of the second loop 2220 is substantially greater than the combined area of the first loop 2218 and the subloops 2222, the secondary subloops 2224 can be included such that the combined magnetic field produced by the first loop 2218, the subloops 2222, and the secondary subloops 2224 substantially balances the magnetic field produced by the second loop 2220. According to various embodiments, each of the secondary subloops 2224 can be substantially nested within one or more of the subloops 2222.

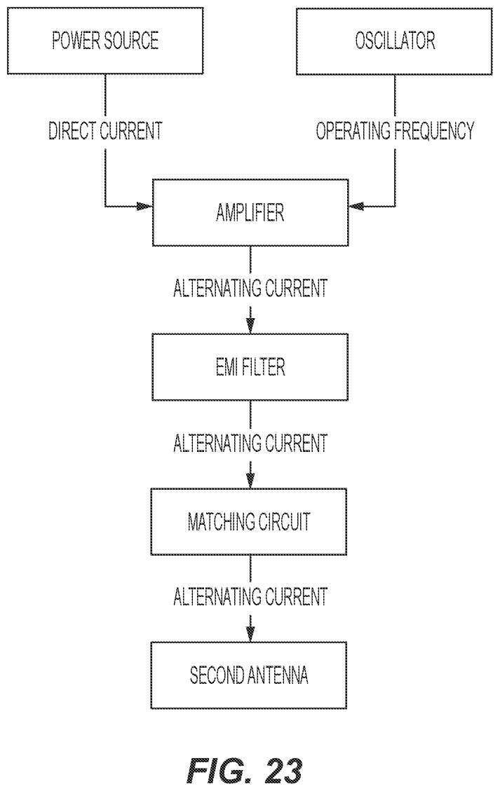

FIG. 23 is a block diagram of a control unit of an external system of the present technology (e.g., control unit 30 of external system 15, etc.) and a second antenna of the external system (e.g., second antenna 12 of external system 15, etc.). The control unit can include and/or be configured to be electrically coupled to a power source for delivering energy to an antenna (e.g., second antenna 12, etc.) of the external system. The control unit can be configured to convert direct current (DC) into alternating current (AC), which can be provided to the second antenna so that the second antenna generates an alternating magnetic field as AC flows through it. As shown in FIG. 23, the control unit can include and/or be configured to be electrically coupled to an amplifier configured to convert DC into AC based on an operating frequency defined by an oscillator. The control unit can include and/or be configured to be electrically coupled to an electromagnetic interference (EMI) filter configured to reduce energy at frequencies other than the operating frequency, for example to comply with radiofrequency emission regulatory requirements. In various embodiments, the control unit includes and/or is configured to be electrically coupled to a matching circuit positioned between the amplifier and/or EMI filter and the second antenna. As described in greater detail below, the matching circuit can facilitate efficient power transfer from the amplifier (e.g., via the EMI filter) to the second antenna.

The power source can comprise any suitable source of DC. In some embodiments, for example, the power source comprises a medical-grade power supply. The power source can be configured to receive AC from a wall outlet, for example, and convert the AC into DC for transmission to the amplifier. In some embodiments, the control unit is configured to be electrically coupled to a medial-grade power supply and includes and/or is configured to be electrically coupled to a programmable power supply. The programmable power supply can be configured to receive DC, for example from the medical-grade power supply and/or other source of DC, and supply DC to the amplifier. The programmable nature of the programmable power supply allows the power supplied to the amplifier to be varied, which is advantageous because the amount of power that needs to be supplied to the amplifier may be based, at least in part, on an impedance of the second antenna. If the impedance of the second antenna differs from the impedance of the amplifier, more power may be required to be supplied to the second antenna (via the amplifier) to generate a magnetic field of a sufficient size and magnitude for powering an implantable device. Moreover, the programmable power supply can be short circuit protected to maintain safety of the control unit if the amplifier has a component failure and/or overheats. In some embodiments, the programmable power supply is configured to measure the amount of power being delivered to the amplifier, which can be used as an input to an algorithm for controlling a matching circuit of the present technology. Additionally or alternatively, measuring the power delivered to the amplifier can be used to enhance the system's safety. It may be desirable, for example, to limit the power delivered to the amplifier to prevent the external system from generating a magnetic field with such a large magnitude that a SAR parameter within a patient using the system exceeds a predetermined safety threshold. Limiting the power delivered to the amplifier can also prevent or limit burnout of components downstream of the amplifier. To minimize or limit heat generation, the programmable power source can have a high efficiency, such as at least 80%, at least 85%, at least 90%, or at least 95%.

The control unit can include and/or be electrically coupled to an oscillator, which can define the operating frequency of the external system. In some embodiments, the operating frequency is defined in accordance with regulatory requirements for electromagnetic radiation. For example, the oscillator can set the operating frequency to a frequency within the Industrial, Scientific, and Medical Equipment Band in accordance with Federal Communications Commission (FCC) regulations. In some embodiments, the operating frequency is 6.78 MHz or 13.56 MHz.

As shown in FIG. 23, the control unit can include and/or be electrically coupled to an amplifier, which can convert DC into AC at the operating frequency of the oscillator. In some embodiments, the amplifier comprises a class D amplifier or a class E amplifier. The EMI filter is configured for reducing electromagnetic noise in the AC generated by the amplifier. For example, the push-pull nature of a class D amplifier can create significant harmonic power from the operating frequency, which the EMI filter can be configured to reduce or eliminate. The EMI filter can comprise an inductor-capacitor (LC) filter comprising one or more inductors and one or more capacitors. In some embodiments, the EMI filter forms a multiple pole low pass filter or a band pass filter. The EMI filter can be configured to at least partially reject energy at offending frequencies outside of the operating frequency (e.g., at harmonics of the operating frequency). Parameters of the EMI filter can be selected so that the EMI filter wastes little power while maximizing or enhancing rejection at the harmonic frequencies. In some embodiments, differential operation reduces energy at even harmonics and so the EMI filter can be configured to reduce energy at odd harmonics (e.g., third harmonic, fifth harmonic, seventh harmonic, eleventh harmonic, etc.).

The EMI filter can comprise capacitors and inductors with small tolerances such that the EMI filter is configured to provide consistent rejection at the harmonics while allowing energy at the operating frequency to pass through the EMI filter with little to no attenuation. In some embodiments, the EMI filter comprises an LC filter augmented with one or more resistor-capacitor (RC) networks placed across the inductors of the filter. The RC network augmented EMI filter can provide enhanced rejection at certain harmonics relative to a standard LC filter. Augmenting an LC filter with one or more RC networks can increase the breadth of the filter.

Referring still to FIG. 23, the control unit can include and/or be electrically coupled to a matching circuit configured to receive AC from the amplifier and/or the EMI filter and provide AC to the second antenna. The matching circuit is configured to enhance power transfer between the amplifier and the second antenna. When a source of power with a fixed output impedance (e.g., the amplifier) operates into a load (e.g., the second antenna), maximum power is delivered to the load when its impedance is equal to the complex conjugate of the impedance of the source. Although the second antenna can be designed to have a natural impedance that is a complex conjugate of the impedance of the amplifier for enhanced power transfer, the impedance of the second antenna can vary over time. For example, the impedance of the second antenna can change if metal objects are placed within the electromagnetic field generated by the second antenna, if a patient lies on the second antenna, if the patient moves relative to the second antenna, etc. To address the foregoing challenges, the matching circuit can be configured to modify an impedance seen by the amplifier and/or the EMI filter (e.g., an impedance of the second antenna) to reduce or eliminate the difference between the impedance of the second antenna and the impedance of the amplifier, thereby enhancing power transfer from the amplifier to the second antenna.

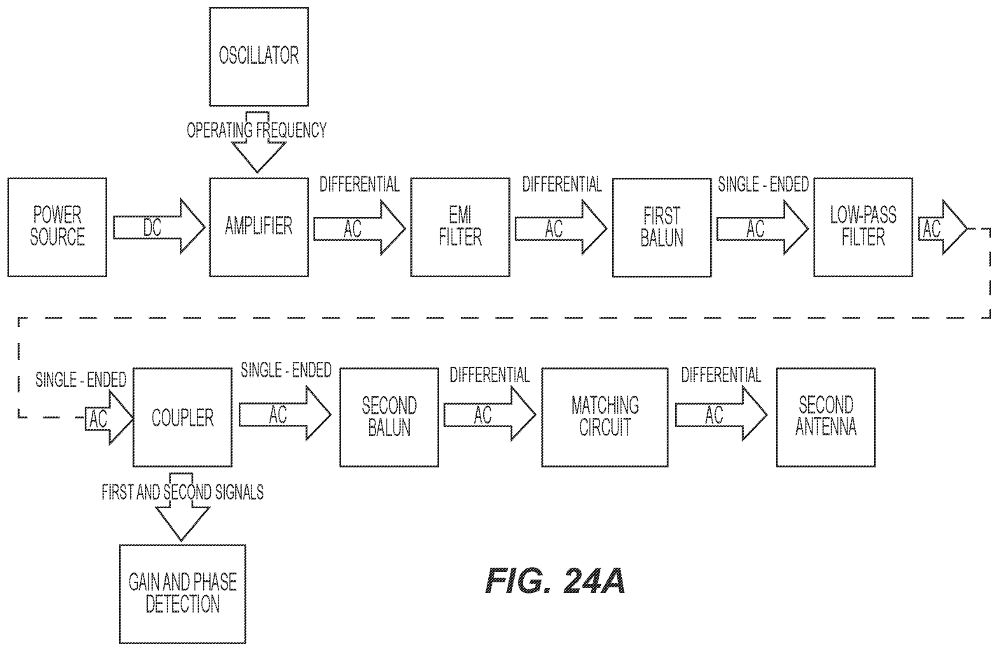

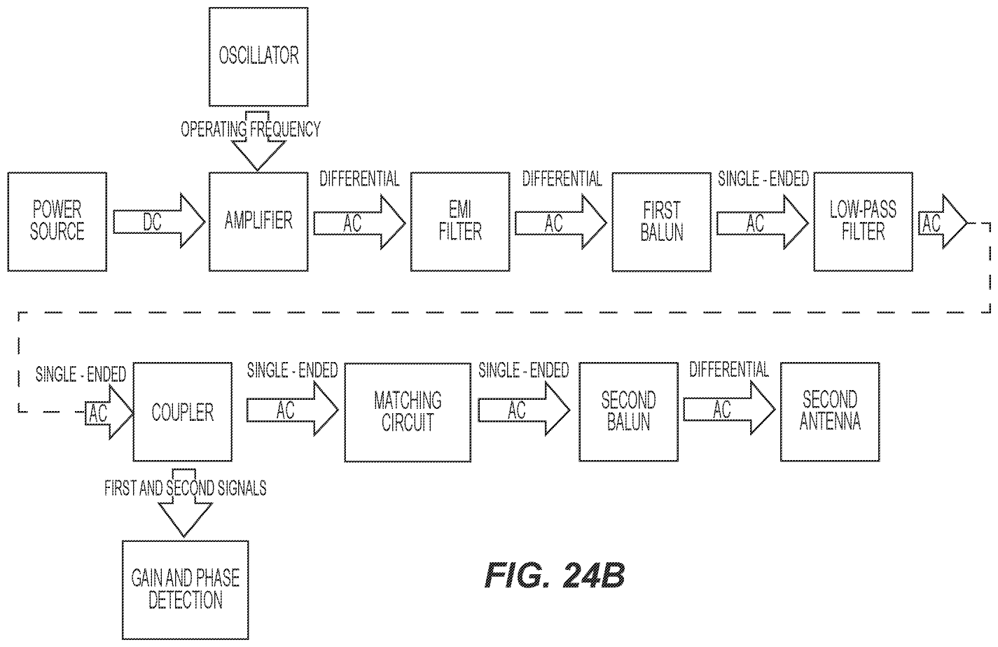

One or more components of a control unit of the present technology can be configured to operate in a differential manner or a single-ended, common ground manner. For example, FIGS. 24A and 24B are block diagrams of control units of the present technology (such as control unit 30) with the matching circuit of the control unit configured for differential operation and single-ended operation, respectively. Components of the control units shown in FIGS. 24A and 24B can have similar features as corresponding components of the control unit described with reference to FIG. 23. For example, as shown in both FIGS. 24A and 24B, the control unit can include and/or be electrically coupled to an amplifier configured to convert DC from a power source to AC based on an operating frequency of an oscillator, as described with reference to the amplifier in FIG. 23. An amplifier of a control unit configured in accordance with various embodiments of the present technology can produce differential AC by driving each side of the amplifier's differential output with a push-pull configuration comprising two field effect transistors (FETs). The on and off times of each of the four FETs (two FETs on each side of the differential output) can be closely controlled to minimize or limit wasted power in the FETs and to enhance power transfer to a second antenna (e.g., second antenna 12). The differential output from the amplifier can be delivered to the EMI filter, which can also produce a differential output.

In some embodiments the control unit can include and/or be electrically coupled to one or more baluns for converting between differential operation and single-ended, common ground operation. For example, as shown in both FIGS. 24A and 24B, the control unit can include a first balun configured to convert from differential operation to single-ended operation and/or a second balun configured to convert from single-ended operation to differential operation. Still, in some embodiments, the control unit can include and/or be configured to be electrically coupled to only one balun to convert the differential output from the amplifier into a single-ended output that is delivered to the second antenna. Components operating in a single-ended manner (e.g., components between the first and second baluns as shown in FIGS. 24A and 24B, components downstream of a single balun, etc.) can advantageously produce less energy at certain harmonics, such as second harmonics. Additionally, the first balun can convert an impedance of the output from the amplifier and/or EMI filter into a standard impedance (e.g., about 50 Ohms, etc.) to facilitate characterization of the output using standard components that operate at the standard impedance. The second balun can convert from the standard impedance to a characteristic impedance of one or more downstream components. The first balun and/or the second balun can have a custom impedance ratio and/or frequency of operation based on specific parameters of other elements of the system, such as a characteristic impedance of the EMI filter, a characteristic impedance of the second antenna, etc.

The control unit can include and/or be configured to be electrically coupled to a low-pass filter configured to reduce noise in the AC generated by the amplifier. As shown in FIGS. 24A and 24B, the low-pass filter can be located downstream of the first balun such that the first balun delivers single-ended AC to the low-pass filter. Alternatively, the low-pass filter can be located upstream of the first balun and, in such examples, could operate differentially. The low-pass filter can be configured to reduce or eliminate energy at frequencies other than the operating frequency defined by the oscillator (e.g., harmonics, etc.). In some embodiments, the low-pass filter is configured to reduce or eliminate energy at certain frequencies that was not reduced or eliminated by the EMI filter. In some embodiments, the low-pass filter has a wide bandwidth such that the low-pass filter is configured to reduce or eliminate energy at high frequencies, which may be more likely to pass through the EMI filter. As but one example, the low-pass filter can have a cutoff frequency of about 10 MHz. The low-pass filter can be configured to output single-ended AC, for example when the low-pass filter is positioned downstream of the first balun.

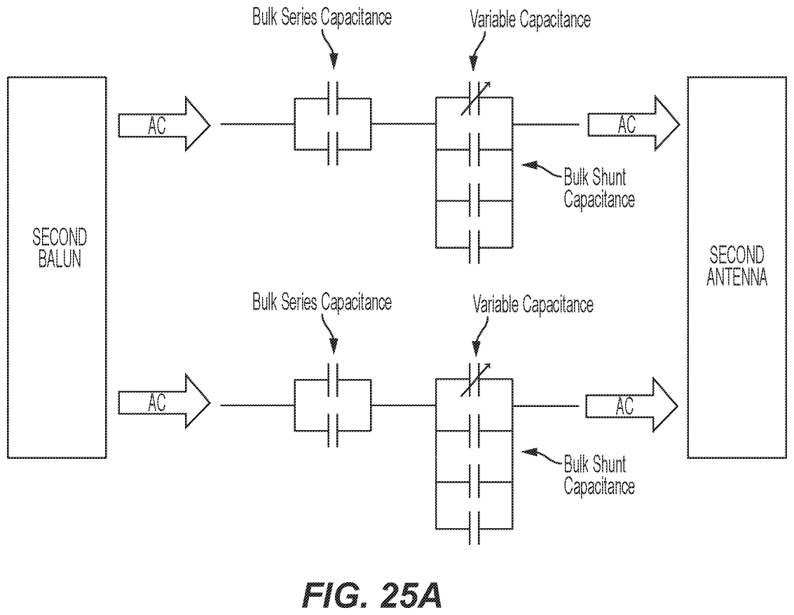

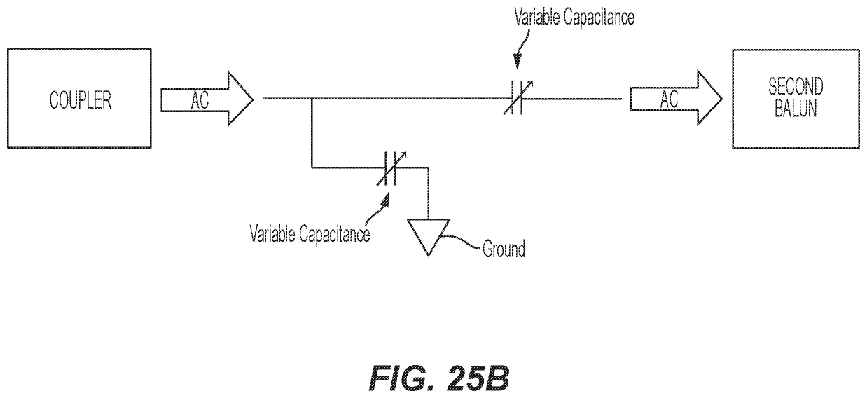

According to various embodiments, for example as shown in FIGS. 24A and 25B, the control unit can include and/or be configured to be electrically coupled to a coupler. The coupler can be positioned downstream of the low-pass filter, the EMI filter, and/or the amplifier. Additionally or alternatively, the coupler can be positioned downstream of the first balun such that the coupler is configured for single-ended operation. In some embodiments, for example when the low-pass filter and coupler are positioned downstream of the first balun, the low-pass filter can be configured to deliver single-ended AC to the coupler. According to various embodiments, the coupler can comprise a directional coupler. In some embodiments, a coupling value of the coupler is about 20 dB. The coupler can be configured to obtain a first signal representing power traveling away from the amplifier toward the second antenna and a second signal representing power traveling away from the second antenna toward the amplifier. As shown in FIGS. 24A and 24B, the coupler can provide the first and second signals to a gain and phase detection unit. In some embodiments, a processor of the control unit can attenuate, filter, AC couple, and/or input the first and second signals to the gain and phase detection unit.

The gain and phase detection unit can be configured to compare the first and second signals to determine a gain relationship and a phase relationship between the first and second signals. The gain relationship can correspond to and/or represent a voltage standing wave ratio (VSWR), which is a measure of how efficiently power is being transferred from the amplifier to the second antenna and thereby how well matched the impedance of the second antenna is to the impedance of the amplifier. The more power being reflected from the second antenna, as represented by the second signal obtained by the coupler, the larger the mismatch in impedance between the second antenna and the amplifier and the less efficiently power is being transferred to the second antenna. The gain relationship can be used to determine a magnitude of impedance change needed at the matching circuit to bring the impedance of the second antenna closer to the impedance of the amplifier. The phase relationship can be useful in determining a direction of impedance change needed at the matching circuit. According to various embodiments, a software algorithm (e.g., executed by a processor of the control unit, etc.) can determine how to modify the matching circuit to minimize or reduce reflected power from the second antenna based on the detected gain relationship and/or phase relationship.

In some embodiments, the second antenna is configured to be driven differentially. Accordingly, as shown in FIGS. 24A and 24B, the control unit can include a second balun configured to convert single-ended AC to differential AC to be delivered to the second antenna. Still, in some embodiments the second antenna is configured to be driven with a single-ended input and the control unit does not include the second balun. In embodiments in which the second antenna is driven differentially, the second antenna can be located downstream of the second balun. As shown in FIG. 24A, the matching circuit can be located downstream of the second balun such that the matching circuit operates differentially. However, as shown in FIG. 24B, in some embodiments the matching circuit is located upstream of the second balun such that the matching circuit operates in single-ended manner.

As previously noted, a matching circuit of the present technology can be configured to alter an impedance seen by the amplifier (e.g., the impedance of a combined circuit comprising the second antenna and the matching circuit) to enhance energy transfer from the amplifier to the second antenna. The matching circuit can present a capacitance and/or inductance on the drive coming from the amplifier (e.g., via the EMI filter), which can be altered to change the natural frequency (e.g., impedance) of the circuit including the second antenna, which is presented to (e.g., seen by) the amplifier. The natural frequency of the circuit including the second antenna can be changed to approach the operating frequency of the system defined by the oscillator, which maximizes or enhances power transfer from the amplifier to the second antenna.

A matching circuit of the present technology can comprise any suitable number and configuration of reactive elements (e.g., inductors and/or capacitors). In some embodiments, the matching circuit comprises one or more programmable capacitive arrays. The programmable capacitive arrays can alter the impedance seen by the amplifier by adding or removing capacitance from the matching circuit. In some embodiments, such capacitive changes can be controlled by a processor (e.g., a processor of the control unit, etc.). Capacitance can be added to or removed from the matching circuit by switching capacitors with different capacitance values into and/or out of the matching circuit. Accordingly, the programmable capacitive arrays can comprise a plurality of capacitors and a plurality of switches for adding or removing the capacitors to or from the matching circuit. The switches can comprise microelectromechanical (MEMS) switches that are driven digitally using software and a processor. MEMS switches may be useful for higher frequency RF applications and may generate few to no harmonics and behave more linearly, even in high power level and high frequency systems. MEMS switches may have a small form factor. In some embodiments, the switches comprise solid state relays (SSRs) in addition to and/or in place of MEMS switches. MEMS switches may not be compatible with “hot switching” in which the switches are activated or deactivated while power is being delivered to the second antenna. In contrast, SSRs are advantageously compatible with hot switching. Accordingly, SSRs can facilitate active tuning of the second antenna while the second antenna is powered and generating an electromagnetic field. Moreover, SSRs may be simpler to implement. In some embodiments, the switches can comprise gallium nitride (GaN) switches, which have a lower capacitance and broader tuning range as compared to silicon SSRs. In addition to or alternatively to switching capacitors in and out of the matching circuit, capacitance can be added to or removed from the matching circuit by changing a value of a tunable capacitor of the matching circuit.

To determine the capacitive change needed in the matching circuit to optimize or enhance power transfer from the amplifier to the second antenna, one or more measurements related to the impedance of the amplifier and/or the second antenna can be obtained. For example, measurements of an alternating magnetic field generated by the second antenna can be used to determine the amount of capacitive change needed in the matching circuit. If a measurement of the alternating magnetic field differs from an expected corresponding measurement, a capacitive change can be implemented by the matching circuit to move the measurement closer to the expected measurement. For example, if a magnitude of the magnetic field generated by the second antenna is smaller than anticipated, the impedance of the second antenna may be mismatched with the impedance of the amplifier such that power is being transferred less efficiently to the second antenna. The capacitance of the matching circuit can then be modified to bring the impedance of the second antenna closer to the impedance of the amplifier. According to various embodiments, one or more portions of an external device (e.g., the carrier 9 of the external device 11, etc.) can include one or more pickup antennas. A pickup antenna can be configured for measuring the alternating magnetic field generated by the second antenna. Each pickup antenna can be distinct from the second antenna. The one or more pickup antennas can be positioned around a periphery of the second antenna and/or can be positioned within the periphery of the second antenna. In some embodiments, each pickup antenna comprises a length of conductive material forming a one turn, two turn, three turn, four turn, five turn, six turn, seven turn, eight turn, nine turn, or ten turn coil. Measurements of the alternating magnetic field generated by the second antenna can be obtained using multiple pickup antennas, and information about the impedance of the second antenna can be determined from such measurements. In some embodiments, an amount and/or type of capacitive change needed at the matching circuit can be determined from the measurements obtained by the multiple pickup antennas.

In some embodiments, an amount of capacitive change needed at the matching circuit can be determined by measuring and evaluating a current going into the amplifier at a given voltage level. When maximum power is being transferred from the amplifier to the second antenna (e.g., when the impedance of the amplifier is matched to the impedance of the second antenna), the current going into the amplifier will also be maximized. Accordingly, the current going into the amplifier can be measured to assess the impedance match between the amplifier and the second antenna, and to determine the amount of capacitive change needed to bring the impedance of the second antenna closer to the impedance of the amplifier.

One or more parameters (e.g., phase, amplitude, etc.) of forward and reflected power can be measured between the amplifier and the second antenna and evaluated to determine a capacitive change to be implemented by the matching circuit. As the impedance of the second antenna diverges from the impedance of the amplifier, a portion of the power that is transmitted from the amplifier to the second antenna is reflected back towards the amplifier. These forward and backward waves interfere with each other to produce standing waves along the transmission line. VSWR can characterize the maximum and minimum voltages of a standing wave and thereby provides insight into any differences in impedance between the amplifier and the second antenna. VSWR can be measured and evaluated to identify an amount of capacitive change needed to reduce the impedance mismatch between the amplifier and the second antenna. In some embodiments, VSWR is measured by a gain and phase detector unit, which can provide information regarding how much capacitive change is needed to match the impedance of the second antenna to the impedance of the amplifier and information regarding the direction in which the capacitance should be changed (e.g., add capacitance, remove capacitance, etc.).

A tuning range of the matching circuit (e.g., the range of change in impedance of the second antenna that can be generated by the matching circuit) can be proportional to an initial impedance of the second antenna, which is at least partially based on the values and locations of capacitors electrically coupled to the second antenna. The tuning range of the matching circuit can be increased by increasing the impedance of the second antenna, which can be accomplished by changing the capacitance values of the capacitors coupled to the second antenna. Increasing the tuning range of the matching circuit is advantageous, because a larger tuning range allows the matching circuit to compensate for larger shifts in the impedance of the second antenna due to changing environmental conditions, loading of the second antenna, etc.

FIGS. 25A and 25B schematically illustrate example matching circuits configured in accordance with various embodiments of the present technology. FIG. 25A depicts a series matching circuit configured for differential operation (e.g., as shown in FIG. 24A). FIG. 25B depicts a series-shunt matching circuit configured for single-ended operation (e.g., as shown in FIG. 24B). Although the series-shunt matching circuit is shown and described with reference to single-ended operation, in some embodiments a matching circuit configured for differential operation (e.g., as shown in FIG. 24A) can comprise a series-shunt matching circuit such as that depicted in FIG. 25B. Operating the series-shunt matching circuit in a differential manner may be less complex than operating in a single-ended manner. However, a matching circuit operating in a single-ended manner requires half the number of matching elements (e.g., capacitors, etc.) as a corresponding matching circuit operating differentially.

FIG. 25A schematically illustrates an example series matching circuit configured for differential operation in accordance with various embodiments of the present technology. As noted with reference to FIG. 24A, AC can be differentially delivered from the amplifier through the EMI filter, the first balun, the low-pass filter, the coupler, and second balun to the matching circuit. Differential AC can be delivered from the matching circuit to the second antenna. Although FIG. 25A illustrates the matching circuit positioned directly between the second balun and second antenna, in various embodiments there may be additional components interspersed between the matching circuit and the second balun and/or the matching circuit and the second antenna. As shown in FIG. 25A, the series matching circuit can comprise two variable capacitances, one on each side of the differential output of the amplifier and EMI filter passed through the second balun. The variable capacitance can include an array of capacitors that can be switched into or out of the matching circuit (e.g., via switches, etc.) to change the overall capacitance of the matching circuit. In some embodiments, the variable capacitance comprises a programmable capacitive array. An array of capacitors can have any suitable configuration, number and/or type of capacitors, number and/or type of switches, etc. Additionally or alternatively, the variable capacitance can include a tunable capacitor whose capacitance can be changed to change the overall capacitance of the matching circuit. Modification of the variable capacitances of the matching circuit can be controlled by a processor of the control unit, in various embodiments.

In some embodiments, a fixed capacitance (e.g., the bulk series capacitance shown in FIG. 25A) can be placed in series between the second balun and each of the variable capacitances. Additionally or alternatively, a fixed capacitance (e.g., the bulk shunt capacitance shown in FIG. 25A) can be placed in parallel with each of the variable capacitances. The bulk series capacitance and/or the bulk shunt capacitance can reduce a voltage across the respective variable capacitance, which enables the use of smaller and less expensive switches in a programmable capacitive array and reduces the risk of switch failure. The bulk shunt capacitance can allow some of the AC to flow around the variable capacitance to reduce dissipation by the variable capacitance, which can also reduce the risk of failure of the switches in a programmable capacitive array. Additionally or alternatively, the bulk shunt capacitance can at least partially resonate the second antenna.

FIG. 25B illustrates an example series-shunt matching circuit configured to operate in a single-ended manner. As previously noted, in some embodiments the series-shunt matching circuit shown in FIG. 25B can be configured to operate in a differential manner. In any case, the series-shunt matching circuit can include one or more variable capacitances in series with the amplifier and/or EMI filter (and any components between the amplifier and/or EMI filter and the matching circuit) and the second antenna (and any components between the second antenna and the matching circuit). For example, as shown in FIG. 25B, a series-shunt matching circuit configured for use in the control unit shown in FIG. 24B can be positioned between the coupler and the second balun. As shown in FIG. 25B, the series-shunt matching circuit can include one or more variable capacitances electrically coupled in series to the amplifier and/or EMI filter (and any components between the amplifier and/or EMI filter and the matching circuit) and shunted to ground. For example, the series-shunt matching circuit can include a variable capacitance downstream of the coupler and shunted to ground. The single-ended output from the variable capacitance not shunted to ground can be transformed by the second balun into a differential output that is delivered to the second antenna. If the second antenna is configured for single-ended operation, the second balun can be omitted and the single-ended output from the variable capacitance can be delivered directly to the second antenna (or delivered to the second antenna via one or more components interspersed between the variable capacitance and the second antenna also configured for single-ended operation). As noted above with reference to FIG. 25A, the variable capacitance can comprise an array of capacitors (e.g., a programmable capacitor array, etc.) and/or a tunable capacitor.

The series-shunt matching circuit may reduce a vulnerability of the second antenna to impedance changes caused by environmental loading of the second antenna (e.g., metal in proximity to the second antenna, physical loading of the second antenna, etc.) relative to the series matching circuit. Additionally, the series-shunt matching circuit may facilitate the use of components with larger impedances, which can result in the generation of less energy at harmonics. Further, the series-shunt matching circuit can facilitate the use of a lower current as compared to the series matching circuit, which can be useful if the variable capacitance comprises a capacitive array including SSRs, GaN switches, and/or MEMS switches. For example, the use of a lower current can prevent or limit switch failures. However, the series matching circuit may have a larger tuning range than the series-shunt matching circuit. Thus, each of the series matching circuit and the series-shunt matching circuits has unique benefits and utility.

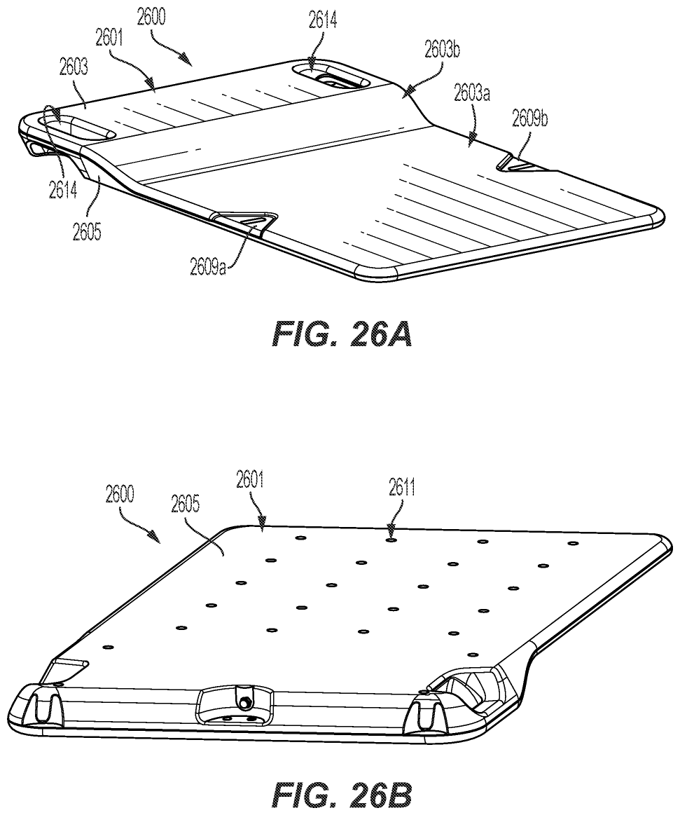

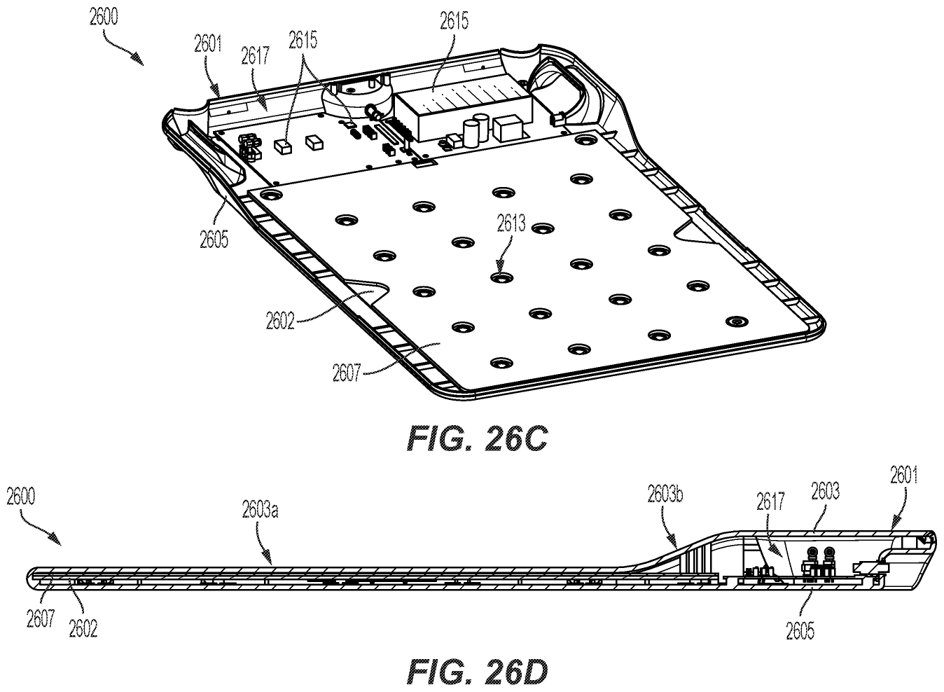

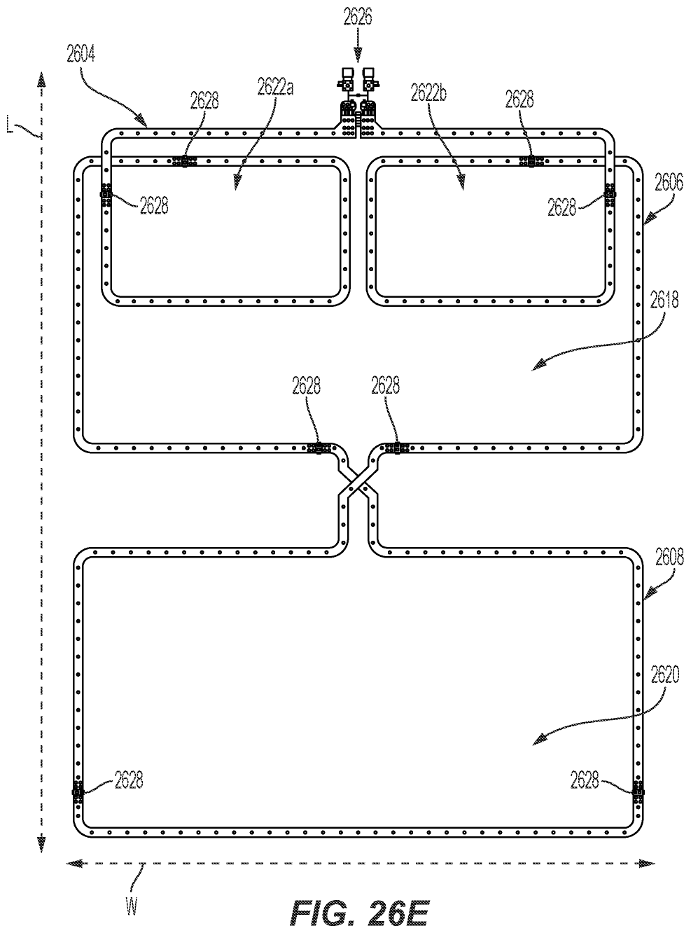

The electronic components disclosed herein (e.g., the amplifier, the matching circuit, etc.) can be physically located within, on, or otherwise secured to one or more portions of a control unit of the present technology (e.g., control unit 30) and/or an external device of the present technology (e.g., external device 11). In some embodiments, for example as described below with reference to FIGS. 26A-26E, an external device can comprise a carrier (e.g., mat) that carries both the second antenna and the control unit. For example, a control unit configured in accordance with the block diagram of FIG. 24A, or a control unit configured in accordance with the block diagram of FIG. 24B, can be located in a mat or other suitable carrier that also includes the second antenna. However, in some embodiments at least a portion of the control unit can be located separate from a carrier that includes the second antenna. For example, a first portion of a control unit (e.g., configured in accordance with the block diagram of FIG. 24A, or in accordance with the block diagram of FIG. 24B) can be located on an external device separate from a carrier including the second antenna, while a second portion of the control unit can be located on the carrier. The first and second portions of the control unit can be operatively coupled with a suitable cable or other connecting device. The cable can, for example, enable the first portion of the control unit to operate while distant from the carrier (e.g., 0.5 meter, 1 meter, 1.5 meters, etc.). In some embodiments, for example, a control unit configured in accordance with the block diagram of FIG. 24B can include a first portion (distant from the carrier) including components upstream of the second balun, and a second portion (incorporated in or near the carrier) including the second balun and all components downstream of the second balun. In some of these embodiments, the second portion of the control unit can include a matching circuit (e.g., the series matching circuit as shown in FIG. 25A).

Any of the features of the external system 15 or one or more components thereof (e.g., the control unit 30, the external device 11, the second antenna 12, etc.) can be varied based on an intended use case of the external system 15. For example, in one use case the external system 15 can be used nightly by a patient over a duration of days, weeks, months, and/or years. Another use case can comprise using the external system 15 in a clinical environment during implantation of the implantable device 100 and/or titration of the stimulation parameters, for example. During implantation and/or titration, the external system 15 can be used to power the implantable device 100 to assess the positioning of the implantable device, evaluate the efficacy of stimulation with one or more specific conductive elements 114 of the implantable device, determine parameters of the stimulation energy to be delivered during treatment, etc. The requirements associated with an at-home use case may differ than the requirements associated with a clinical use case and thus, one or more features of the external system 15 may vary based on the intended use case. Still, in some embodiments, an external system 15 configured for at-home use can have similar features as an external system 15 configured for clinical use.

FIGS. 26A-26E illustrate an example of an external device 2600. In some embodiments, the external device 2600 can be configured for use in a clinical environment. Features of the external device 2600 can be generally similar to the features of the external device 11 of FIG. 2. Any of the features of the external device 2600 of FIGS. 26A-26E can be combined with each other and/or with the features of the external device 11 of FIG. 2 and any of the features of the external device 11 of FIG. 2 can be combined with any of the features of the external device 2600 of FIGS. 26A-26E. Moreover, although the external device 2600 is described with reference to use in a clinical environment, the external device 2600 can be used in any environment or use case (e.g., at home, etc.).

The external device 2600 can comprise a carrier 2601 enclosing a substrate 2602 carrying an antenna 2604. FIGS. 26A and 26B are perspective views of the carrier 2601 of the external device 2600, FIGS. 26C and 26D are cutaway views of the external device 2600, and FIG. 26E is a planar view of the antenna 2604 of the external device 2600.

As shown in FIGS. 26A and 26B, in some embodiments the carrier 2601 comprises an upper portion 2603 and a lower portion 2605. The upper and lower portions 2603, 2605 can be integral with one another, permanently secured to one another, and/or releasably secured to one another. Abutting edges of the upper and lower portions 2603, 2605 can be bonded with adhesive, welded, mechanically fastened, or otherwise secured to one another. The lower portion 2605 can be configured to be positioned on a surface beneath a patient (e.g., an operating table, an examination table, a sleeping surface, etc.). At least one region of the upper portion 2603 can be configured to be positioned between the lower portion 2605 of the carrier 2601 and the patient. As shown in FIG. 26B, the lower portion 2605 can be substantially flat. In some embodiments, the lower portion 2605 of the carrier 2601 defines one or more apertures 2611 (see FIG. 26B) each configured to receive a fastener therein for securing the carrier 2601 to the substrate 2602.

In some embodiments, for example as shown in FIG. 26A, the upper portion 2603 can comprise a substantially flat region 2603a and a ramped region 2603b. As shown in FIG. 26C, the substrate 2602 carrying the antenna 2604 can be positioned at the substantially flat region 2603a. The flat region 2603a and thereby the substrate 2602 and antenna 2604 can be configured to be positioned between the lower portion 2605 of the carrier 2601 and the patient. The ramped region 2603b and the lower portion 2605 of the carrier 2601 define an interior volume 2617 that is larger than an interior volume defined by the substantially flat region 2603a of the upper portion 2603 and the lower portion 2605 and/or has a larger cross-sectional area than a cross-sectional area defined by the substantially flat region 2603a. One or more electronic components 2615 (e.g., a control unit, a power source, etc.) can be positioned within the interior volume 2617 (see FIGS. 26C and 26D). The ramped region 2603b can be configured to be positioned proximate a patient but not underneath the patient during use. For example, the substantially flat region 2603a can be configured to be positioned between a patient's head and a surface beneath the patient while the ramped region 2603b is positioned laterally or superiorly of the patient's head.

Although FIGS. 26A-26D illustrate the device 2600 comprising a single carrier 2601 for carrying the substrate 2602 and electronic components 2615, in some embodiments the device 2600 comprises multiple, distinct carriers, as noted above. For example, the device 2600 can comprise a first carrier 2601 carrying the substrate 2602 and a second, separate carrier 2601 carrying the electronic components 2615. The antenna 2604 can be electrically coupled to the electronic components 2615 via a connector extending between the first and second carriers, as previously described. Separating the antenna 2604 from the electronic components 2615 may allow the electronic components 2615 to be positioned further away from the patient's head and/or body, thus preventing or limiting heat transfer from the electronics components 2615 to the patient. Moreover, each of the distinct carriers 2601 can comprise a distinct material based on the specific requirements of the individual carrier.

In some embodiments, the device 2600 can include cushioning. For example, cushioning can be carried by the carrier 2601 of the device so that, when the device 2600 is positioned proximate the patient during use, the cushioning is positioned between the carrier 2601 and the patient's head. While the patient may be asleep during use of the device 2600, the patient may still experience discomfort once awake if the patient has been lying on a hard surface for an extended time. Accordingly, the cushioning can have a low hardness parameter to improve patient comfort. The cushioning can be carried by the substantially flat region 2603a and/or the ramped region 2306b. The cushioning can comprise any sufficiently soft material, such as one or more foams. Additionally or alternatively, the cushioning can be configured to dissipate heat from the patient's head.

The external device 2600 can include one or more manipulation portions 2614 configured to facilitate manipulation of the external device 2600 by a user. For example, as shown in FIG. 26A, the manipulation portions 2614 can comprise apertures in the carrier 2601 that form handles that can be grasped by a user. In a clinical environment in which the external device 2600 is used on an anesthetized patient, use of the manipulation portions 2614 can allow a clinician to more easily grasp the external device 2600 and reposition the external device 2600 relative to the patient.

The carrier 2601 of the external device 2600 can comprise a material including, for example, polycarbonate, polymethyl methacrylate, acrylonitrile butadiene styrene, nylon, polylactic acid, polyethylene, polypropylene, polystyrene, polysulfone, polyethersulfone, and/or others. The carrier 2601 can be substantially rigid when configured for use in a clinical setting. If an external device is intended for nightly use by a patient, it is advantageous for the substrate and/or carrier of the device to be flexible and/or soft for the patient's comfort. However, in a clinical setting the external device 2600 is intended for use over a short duration with each patient and the patient may be anesthetized during use of the device. Accordingly, comfort requirements are less stringent when the external device 2600 is configured for clinical use. Further, in a clinical environment it can be useful for the carrier 2601 to comprise a material that is resistant to fluid ingress and/or easy to clean to facilitate use of the device with multiple patients. In some embodiments, the carrier 2601 can comprise a material with good heat resistance to prevent or limit deformation of the substrate 2602 during use of the external device 2600.

The external device 2600 can be configured for use during a procedure in which an implantable device is implanted in a patient's head, which may occur in an operating room while the patient lies on an operating table. Operating tables often comprise a substantial amount of metal and/or other materials such as carbon fiber that can modify an impedance of the antenna 2604. To prevent or limit the operating table from changing an impedance of the antenna 2604, the external device 2600 can include a shielding material. The shielding material can be positioned between the antenna 2604 and lower portion 2605 of the carrier 2601 and/or can be configured to be positioned between the lower portion 2605 of the carrier 2601 and the operating table.

In some embodiments, the antenna 2604 is driven with more power when used in an operating room relative to an at-home setting. Driving the antenna 2604 with additional power can compensate for additional detuning and/or preloading of the antenna 2604 that may occur in the operating room. When higher power is used to drive the antenna 2604, the control unit and/or the antenna 2604 may produce more heat. To address this concern, the external device 2600 can include an insulation 2607 that modifies and/or controls heat dissipation from the antenna 2604. As shown in FIGS. 26C and 26D, the insulation 2607 can be positioned between the antenna 2604 and the upper portion 2603 of the carrier 2601 to prevent or limit heat transfer from the antenna 2604 to the patient. Insulation 2607 can be provided at any other suitable location within and/or on the carrier 2601 in addition to or in place of the insulation 2607 shown in FIGS. 26C and 26D. In some embodiments, the insulation 2607 comprises a substantially flat sheet. The insulation 2607 can define one or more apertures 2613 (see FIG. 26C) each configured for receiving a fastener therethrough for securing the insulation 2607 to the carrier 2601 via the fasteners. In various embodiments, the insulation 2607 does not include apertures 2613. The insulation 2607 can comprise synthetic fibers with high heat resistance such as aramid fibers, for example, and/or another suitable felt or insulative material. The insulation 2607 can be resistant to moisture absorption, in some embodiments.

The substrate 2602 is positioned between the upper portion 2603 and the lower portion 2605 of the carrier 2601. In some embodiments, the substrate 2602 is positioned between the lower portion 2605 and the insulation 2607. The substrate 2602 can comprise a printed circuit board (PCB) substrate. For example, the substrate 2602 can comprise FR4, CEM1, CEM3, FR2, PET, elastomers, and/or another suitable PCB substrate. The substrate 2602 can comprise a dielectric material with good heat resistance. In some embodiments, a substrate configured for use in a clinical setting is more rigid than a substrate configured for use in an at-home setting. The substrate 2602 can define one or more apertures 2616 extending therethrough. Similar to the apertures 2613 of the insulation 2607, the apertures 2616 of the substrate 2602 can each be configured to receive a fastener therein for securing the substrate 2602 to the carrier 2601. The apertures 2616 can be configured to receive columns, for example, of the carrier 2601 to secure the carrier 2601 to the substrate 2602. In some embodiments, the apertures 2613 can be configured to be bonded, adhered, welded, or otherwise fixed to such columns. According to various embodiments, the substrate 2602 can define the same number of apertures 2616 as the number of apertures 2613 defined by the insulation 2607 and/or the apertures 2616 of the substrate 2602 can be configured to align with the apertures 2613 of the insulation 2607. As a result, one fastener can extend through an aperture 2616 of the substrate 2602 and an aperture 2613 of the insulation 2607.

A fastener configured to extend through the apertures 2611 of the carrier 2601, the apertures 2613 of the insulation 2607, and/or the apertures 2616 of the substrate 2602 can comprise a screw, a post, a column, a nail, or any other suitable fastener. According to various embodiments, the carrier 2601 can include the fastener. For example, the fastener can be monolithic with the carrier 2061. The fastener can comprise a material that is not electrically or magnetically conductive to avoid interference with the antenna 2604. As but one example, the fastener can comprise a polymer such as, but not limited to, polyetheretherketone. In some embodiments, one or more of the apertures 2611, 2613, 2616 can be configured to receive a grommet therein, which may comprise an elastomeric material.

In some embodiments, the carrier 2601 can be configured to be bonded to the substrate 2602 and/or the insulation 2607. Bonding the carrier 2601 to the substrate 2602 and/or the insulation 2607 can enhance a strength and rigidity of the device 2600. For example, bonding the substrate 2602 to the carrier 2601 can prevent or limit deformation of the substrate 2602, which can in turn prevent or limit unintentional changes being induced in the magnetic field generated by the antenna 2604.

As shown in FIG. 26E, the antenna 2604 can have similar features as other antennas disclosed herein (e.g., second antenna 12, antennas 404, 1004, 1204, 1504, 1804, 1904, 2004, 2104, 2204, etc.). For example, the antenna 2604 can comprise a first length 2606 of conductive material forming a first loop 2618, a first subloop 2622a, and a second subloop 2622b and a second length 2608 of conductive material forming a second loop 2620. Additionally, the antenna 2604 can include a feed 2626 at which the antenna 2604 is configured to electrically couple to a control unit and one or more capacitors 2628 at the feed 2626, proximate the feed 2626, and/or along the first and/or second lengths 2606, 2608.

The antenna 2604 can have a width based at least in part on a width of a surface upon which the antenna 2604 is configured to be positioned within the clinical setting. In many cases, operating tables are narrow (e.g., 50 cm in width as compared to 97 cm in width of a twin size bed). Accordingly, the antenna 2604 can have a smaller width when configured for use in a clinical setting. For example, while an antenna configured to be used at home by a patient can have a width of about 20 cm to about 100 cm, for example about 70 cm, an antenna configured for use in a clinical setting (e.g., antenna 2604) can have a width of about 40 cm to about 55 cm, for example about 42 cm. The antenna 2604 can have a width of less than 50 cm when configured to be positioned on an operating table having a width of 50 cm. In some embodiments, a width of the antenna 2604 is about 50 cm, about 48 cm, about 46 cm, about 44 cm, about 42 cm, about 40 cm, about 38 cm, about 36 cm, about 34 cm, about 32 cm, or about 30 cm.

The antenna 2604 can be configured to generate electromagnetic field with a smaller active volume when configured to be used in a clinical setting. During normal sleep, a patient may move such that the of the implantable device in the patient's head moves relative to the antenna of the external device. However, if the patient is anesthetized and/or restrained (e.g., in an operating room, during implantation, etc.), there will be little to no movement of the patient relative to the external device. Thus, the position of the implantable antenna relative to the external antenna should remain relatively constant during use of the external device 2600.

To facilitate powering the implantable antenna with an electromagnetic field having a smaller active volume, the carrier 2601 of the external device 2600 can comprise one or more markings configured to facilitate positioning of the patient on/over the external device 2600. For example, FIG. 26A depicts the carrier 2601 comprising a first marking 2609a and a second marking 2609b (collectively “markings 2609”), which indicate a region of the external device 2600 at which an anatomical landmark of a patient (e.g., a chin, a nose, an ear, etc.) should be positioned. The markings 2609 can comprise a recessed portion of the carrier 2601, a protruding portion of the carrier 2601, a discrete element secured to the carrier 2601, a printed material disposed on the carrier 2601, etc.

In some embodiments, a predetermined threshold for a SAR parameter within a patient during use of the external device is greater for a system configured to be used in a clinical environment than for a system used at home by a patient on a recurrent basis. In a clinical environment, the controlled exposure regulations may apply. Under the controlled exposure regulations, a peak spatial-average SAR averaged over any 1 gram of tissue, except at extremities of the patient and averaged over no more than 6 minutes has a predetermined threshold of 8 W/kg, a peak spatial-average SAR averaged over any 10 grams of tissue at extremities of the patient and averaged over no more than 6 minutes has a predetermined threshold of 20 W/kg, and/or an average SAR averaged over an entire body of the patient and averaged over no more than 6 minutes has a predetermined threshold of 0.4 W/kg.

### CONCLUSION

Although many of the embodiments are described above with respect to systems, devices, and methods for modulation of a hypoglossal nerve of a patient, the technology is applicable to other applications and/or other approaches, such as modulation of other nerves of a patient. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1A-26E.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A device for use with an implant implanted in a patient at a first anatomical region, the device comprising:
a carrier configured to be positioned on a surface, the carrier having non-overlapping first and second regions; and
an antenna carried by the carrier and configured to generate a magnetic field that is denser at the first region of the carrier than the second region, wherein the magnetic field is configured to power the implant when the carrier is positioned on the surface and proximate the patient such that the first region of the carrier is aligned with the first anatomical region and the second region of the carrier is aligned with a second anatomical region of the patient, the second anatomical region having a greater soft tissue to bone ratio than the first anatomical region, and wherein, when the magnetic field is powering the implant, a specific absorption rate (SAR) parameter within the patient tissue does not exceed a predetermined threshold.

2. The device of claim 1, wherein the first region of the carrier exhibits a greater amount of magnetic flux from the antenna than the second region of the carrier.

3. The device of claim 1, wherein the magnetic field has a component that is configured to extend through an antenna of the implant in a direction substantially perpendicular to a radial dimension of the antenna of the implant across a range of nod angles, axial head angles, head positions, and/or head rotations.

4. The device of claim 3, wherein the component of the magnetic field is substantially perpendicular to the surface.

5. The device of claim 1, wherein the first anatomical region comprises a head of the patient and the second anatomical region is inferior of the head of the patient.

6. The device of claim 1, wherein the carrier is configured to be positioned such that the second anatomical region is positioned closer to the carrier than the first anatomical region along a dimension substantially perpendicular to the surface.

7. The device of claim 1, wherein the first region of the antenna comprises a first length of conductive material forming at least a first loop and a second loop and the second region of the antenna comprises a second length of conductive material forming a single loop.

8. The device of claim 7, wherein the second loop is contained within an internal area defined by first loop.

9. The device of claim 7, wherein the first loop is electrically coupled to the second loop in series.

10. The device of claim 7, wherein the antenna comprises a transition region comprising:
a first segment comprising a first end portion of the first length and a first end portion of the second length, wherein the first segment is configured to carry RF current in a first direction; and
a second segment comprising a second end portion of the first length and a second end portion of the second length, wherein the second segment is configured to carry RF current in a second direction opposite the first direction,
wherein at least a portion of the first segment and at least a portion of the second segment overlap along a thickness dimension of the antenna in the transition region.

11. The device of claim 10, wherein the first and second segments are positioned at an angle of less than about 30 degrees relative to each other within a plane substantially perpendicular to the thickness dimension.

12. The device of claim 10, wherein the first and second segments are aligned along the thickness dimension of the carrier.

13. The device of claim 1, wherein the first region of the carrier is substantially coplanar with the second region of the carrier.

14. The device of claim 1, wherein the SAR parameter comprises a peak spatial-average SAR averaged over any 1 gram of tissue of the patient, except at extremities of the patient, and averaged over no more than 30 minutes, and wherein the predetermined threshold comprises 1.6 W/kg.

15. A device for use with an implant implanted in a patient at a first anatomical region, the device comprising:
a carrier configured to be positioned on a surface, the carrier having non-overlapping first and second regions; and
an antenna carried by the carrier and comprising a conductive material having a first length in a first configuration with one or more loops within the first region and a second length in a second configuration with one or more loops within the second region,
wherein the first and second configurations include different amounts of the conductive material, and
wherein the antenna is configured to produce a magnetic field configured to power the implant when the carrier is positioned on the surface and proximate the patient such that the first region of the carrier is aligned with the first anatomical region and the second region of the carrier is aligned with a second anatomical region of the patient, and wherein, when the magnetic field is powering the implant, a specific absorption rate (SAR) parameter within the patient does not exceed a predetermined threshold.

16. The device of claim 15, wherein the first anatomical region comprises a head of the patient and the second anatomical region is inferior of the head of the patient.

17. The device of claim 15, wherein the carrier is configured to be positioned such that the second anatomical region is positioned closer to the carrier than the first anatomical region along a dimension substantially perpendicular to the surface.

18. The device of claim 15, wherein the first region of the carrier comprises a first density of conductive material and the second region of the carrier comprises a second density of conductive material less than the first density of conductive material.

19. The device of claim 18, wherein the first and second configurations differ in at least one of length, number of loops, or size of loops.

20. The device of claim 18, wherein the first region of the carrier exhibits a greater amount of magnetic flux from the antenna than the second region of the carrier.

21. The device of claim 15, wherein the antenna comprises:
a first segment comprising a first end portion of the first length and a first end portion of the second length, wherein the first segment is configured to carry RF current in a first direction; and
a second segment comprising a second end portion of the first length and a second end portion of the second length, wherein the second segment is configured to carry RF current in a second direction opposite the first direction,
wherein the first segment and the second segment overlap along a thickness dimension of the carrier.

22. The device of claim 21, wherein the first and second segments are positioned at an angle of less than about 30 degrees relative to each other within a plane substantially perpendicular to the thickness dimension.

23. The device of claim 21, wherein the first and second segments are aligned along the thickness dimension of the carrier.

24. A device for use with an implant implanted in a patient at a first anatomical region, the device comprising:
a carrier configured to be positioned on a surface, the carrier having a first side and a second side opposite the first side of the carrier, wherein the carrier comprises a first region between the first side and a midline and a second region between the second side and the midline, and wherein current flows through the first region in a first direction and current flows through the second region in a second direction opposite the first such that the flow of current through the antenna reverses at the midline; and
an antenna carried by the carrier and comprising conductive material forming at least two first loops at the first region of the carrier and a second loop at the second region of the carrier, wherein the antenna is configured to produce a magnetic field configured to power the implant when the carrier is positioned on the surface and proximate the patient such that the first region of the carrier is aligned with the first anatomical region and the second region of the carrier is aligned with a second anatomical region of the patient, wherein, when the magnetic field is powering the implant, a specific absorption rate (SAR) parameter within the patient does not exceed a predetermined threshold.

25. The device of claim 24, wherein the at least two first loops are electrically coupled in series with the second loop and with one another.

26. The device of claim 24, wherein electrical current is configured to flow through each of the at least two first loops in a first direction and electrical current is configured to flow through the second loop in a second direction opposite the first direction.

27. The device of claim 24, wherein the at least two first loops comprise a major loop and at least two minor loops, and wherein the major loop encloses a first area and each of the at least two minor loops encloses a second area less than the first area.

28. The device of claim 27, wherein the at least two minor loops are spaced apart from one another along a length dimension of the carrier, the length dimension of the carrier being substantially perpendicular to the width dimension.

29. The device of claim 27, wherein the antenna is configured to operate at a frequency of about 6.78 MHz.

30. The device of claim 24, wherein the first region of the antenna comprises a first length of conductive material forming at least a first loop and a second loop and the second region of the antenna comprises a second length of conductive material forming a single loop, wherein the antenna comprises:
a first segment comprising a first end portion of the first length and a first end portion of the second length, wherein the first segment is configured to carry current in the first direction; and
a second segment comprising a second end portion of the first length and a second end portion of the second length, wherein the second segment is configured to carry current in the second direction,
wherein the first segment and the second segment overlap along a thickness dimension of the carrier.

31. The device of claim 30, wherein the first and second segments are positioned at an angle of less than about 30 degrees relative to each other within a plane substantially perpendicular to the thickness dimension.

32. The device of claim 30, wherein the first and second segments are aligned along the thickness dimension of the carrier.
